(19) United States
(12) Patent Application Publication
(10) Pub. No.: US 2025/0250576 A1
(43) Pub. Date: Aug. 7, 2025

(54) METHODS AND COMPOSITIONS FOR TREATING ATHEROSCLEROSIS

(71) Applicant: Yale University, New Haven, CT (US)

(72) Inventors: Michael Simons, New Haven, CT (US); Pei-Yu Chen, New Haven, CT (US)

(21) Appl. No.: 19/009,740

(22) Filed: Jan. 3, 2025

Related U.S. Application Data
(63) Continuation of application No. 17/721,666, filed on Apr. 15, 2022, now abandoned.
Application No. 17/721,666 is a continuation of application No. 16/086,809, filed on Sep. 20, 2018, now Pat. No. 11,326,167.
(60) Provisional application No. 62/311,086, filed on Mar. 21, 2016.
(60) Provisional application No. 62/406,732, filed on Oct. 11, 2016.

(51) Int. Cl. C12N 15/113; A61K 9/00; A61K 9/51; A61K 31/436; A61P 9/10; C12Q 1/68

(52) U.S. Cl. CPC C12N 15/1138; A61K 9/0019; A61K 9/51; A61P 9/10; C12N 15/113; C12Q 1/68; A61K 31/436; C12N 2310/14; C12N 2310/141; C12N 2310/321; C12N 2320/32

(57) ABSTRACT

In some aspects, the invention provides a method of treating atherosclerosis in a subject. The method comprises administering to the subject an agent that increases the activity or level of a let-7 miRNA or an agent that decreases activity or level of a TGFβ signaling polypeptide in an endothelial cell in the subject. In some embodiments, the subject is administered an additional agent comprising a therapeutically effective amount of rapamycin or any derivative thereof. In some embodiments, the agent is a let-7 miRNA. In some other aspects, the invention provides a pharmaceutical composition comprising a let-7 miRNA. In some embodiments, the let-7 miRNA is encapsulated in a nanoparticle formulated for selective delivery to an endothelial cell.

### CROSS-REFERENCE TO RELATED APPLICATIONS

This present application is a continuation of U.S. Non-Provisional patent application Ser. No. 17/721,666, filed Apr. 15, 2022, which is a continuation of U.S. Non-Provisional patent application Ser. No. 16/086,809, filed Sep. 20, 2018, now issued as U.S. Pat. No. 11,326,167 on May 10, 2022, which is a 35 U.S.C. § 371 national phase application from, and claims priority to, International Application No. PCT/US2017/023347, filed Mar. 21, 2017, and published under PCT Article 21(2) in English, which claims priority under 35 U.S.C. § 119(e) to U.S. Provisional Patent Application No. 62/311,086, filed Mar. 21, 2016 and U.S. Provisional Patent Application No. 62/406,732, filed Oct. 11, 2016, which are incorporated herein by reference in their entirety.

### STATEMENT REGARDING FEDERALLY SPONSORED RESEARCH OR DEVELOPMENT

This invention was made with government support under HL053793 and HL107205 awarded by National Institutes of Health. The government has certain rights in the invention.

### REFERENCE TO SEQUENCE LISTING

The Sequence Listing concurrently submitted herewith as a xml file named “047162-7080US3 Sequence Listing” created on Jan. 3, 2025 and having a size of 65.7 Kbytes is herein incorporated by reference.

### BACKGROUND OF THE INVENTION

Atherosclerosis is responsible for the vast majority of cardiovascular diseases. Despite decades of work, statins remain the only effective therapy, but they can only slow, not stop or reverse disease progression. There is no currently available therapy to stop the development of atherosclerosis and induce its regression.

In healthy mature blood vessels, vascular smooth muscle cells (SMCs) are quiescent, fully differentiated cells that exhibit a very low rate of proliferation. They express a number of contractile proteins necessary for maintaining vessel tone, blood pressure, and blood flow, including smooth muscle α-actin (SM α-actin), smooth muscle 22 alpha (SM22α), SM-calponin, and smooth muscle myosin heavy chain (SM-MHC) (Liu et al, 2015 Biochimica et biophysica acta 1849: 448-453; Owens et al, 2004, Physiological reviews 84: 767-801; Shi & Chen, 2014, Journal of biomedical research 28: 40-46). Following vascular injury or in association with a variety of diseases, SMCs exhibit a decrease in expression of differentiation markers and acquire a proliferative phenotype characterized by enhanced cell proliferation and migration (Kawai-Kowase & Owens, 2007, Cell physiology 292: C59-69; Owens et al, 2004, Physiological reviews 84: 767-801). This form of SMC phenotypic modulation is especially robust in atherosclerosis and vascular stenosis following angioplasty where it is thought to contribute to the growth of atherosclerotic plaques and neointima (Gomez & Owens, 2012, Cardiovascular research 95: 156-164; Marx et al, 2011, Circulation Cardiovascular interventions 4: 104-111; Tabas et al, 2015, J Cell Biol 209: 13-22). Therefore, elucidation of mechanisms that control normal SMC phenotypic switch in disease states is likely to provide key insights toward understanding the biology of atherosclerosis and development of new therapeutic targets.

Smooth muscle differentiation is promoted by a number of signaling pathways including transforming growth factor β (TGFβ), Notch3 as well as integrin- and extracellular matrix-derived differentiation signals. TGFβ signaling is particularly critical for the maintenance of normal adult vasculature (Li et al, 2014, Journal of clinical investigation 124: 755-767) and the growth factor plays a critical role in mediating balance between inflammation and fibrous plaque growth in atherosclerosis (Lutgens et al, 2002, Arterioscler Thromb Vasc Biol 22: 975-982). TGFβ exerts its effects via a complex of two serine/threonine kinase type II receptors (TGFβR2) and the type I receptor Alk5 (TGFβR1) (Carvalho et al, 2007, Journal of cell science 120: 4269-4277; Mack, 2011, Arterioscler Thromb Vasc Biol 31: 1495-1505). TGFβR1 phosphorylation by TGFβR2 results in recruitment and phosphorylation of Smad2 and Smad3 that then complex with Smad4 and translocate to the nucleus. Subsequent activation of contractile SMC-specific gene expression involves both direct binding of Smads to certain DNA binding sites as well as interactions with other SMC transcription factors such as SRF and myocardin. TGFβ also activates non-Smad-dependent signaling pathways that also play a role in the induction of SMC differentiation (Li et al, 2014, Journal of clinical investigation 124: 755-767). In agreement with these results, genetic deletions of either TGFβ 1, TGFβ2, their receptors (TGFβR1, TGFβR2) or signaling molecules (Smad2, Smad3), are all associated with various vascular wall pathologies including aneurysm formation (Carvalho et al, 2007, Journal of cell science 120: 4269-4277; Crosas-Molist et al, 2015, Arterioscler Thromb Vase Biol 35: 960-972; Doyle et al, 2012, Nature genetics 44: 1249-1254; Li et al, 2014, Journal of clinical investigation 124: 755-767; Lindsay et al, 2012, Nature genetics 44: 922-927; Tang et al, 2010, Journal of biological chemistry 285: 17556-17563).

While the central role played by TGFβ in regulation of SMC differentiation has been previously demonstrated (Hirschi et al, 1998, J Cell Biol 141: 805-814; Kawai-Kowase et al, 2004, Arterioscler Thromb Vasc Biol 24: 1384-1390; Lindner & Reidy, 1991, Proc Natl Acad Sci USA 88: 3739-3743), little is known about what regulates this pathway and what contribution SMC proliferation makes to progression of lesions seen in atherosclerosis (Tabas et al, 2015, J Cell Biol 209: 13-22). Recent studies in endothelial cells demonstrated FGF-dependent regulation of TGFβ. The loss of endothelial cell FGF signaling input in vitro or in vivo leads to a profound decrease in let-7 miRNAs levels that results in marked prolongation of TGFβR1 mRNA half-life and increased TGFβR1 protein expression. Together with a large increase in TGFβ2 levels, this leads to activation of TGFβ signaling including phosphorylation of Smad2 and Smad3 and induction of expression of various smooth muscle and mesenchymal markers thereby inducing endothelial-to-mesenchymal transition (EndMT) (Chen et al, 2012, Cell reports 2: 1684-1696; Chen et al, 2014, Science signaling 7: ra90). Importantly, EndMT, in turn, leads to acceleration of atherosclerosis progression (Chen et al, 2015, Journal of clinical investigation 125: 4529-4543). Prior studies also reported FGF antagonism of TGFβ activity in SMCs and pericytes in vitro but the mechanism of this effect and its functional consequences have not been fully established. (Kawai-Kowase et al, 2004, Arterioscler Thromb Vasc Biol 24: 1384-1390; Papetti et al, 2003, Investigative ophthalmology & visual science 44: 4994-5005).

New methods of treating atherosclerosis, particularly methods of inhibiting development or progression and methods of reversing atherosclerosis that target the molecular events that drive progression of atherosclerosis, are urgently needed.

### SUMMARY OF THE INVENTION

In one aspect, the invention comprises a pharmaceutical composition comprising an effective amount of a let-7 miRNA in a nanoparticle formulated for selective delivery to an endothelial cell, in a pharmaceutically acceptable excipient. In various embodiments, the let-7 miRNA comprises a chemical modification that increases stability of the miRNA and/or reduces an immune response to the miRNA in a subject. In various embodiments, the chemical modification is a 2′-O-methyl modification. In various embodiments, the let-7 miRNA is selected from the group consisting of human let-7b miRNA and human let-7c miRNA. In various embodiments, the nanoparticle is a 7C1 nanoparticle.

In another aspect, the invention comprises a method of reducing an atherosclerotic lesion in a subject, the method comprising administering to the subject an agent that modulates the activity or level of let-7 miRNA in an endothelial cell in the subject, thereby reducing or inhibiting the atherosclerotic lesion in the subject.

In another aspect, the invention comprises a method of reducing an atherosclerotic lesion in a subject, the method comprising administering to the subject an agent that decreases in an endothelial cell in the subject the activity or level of a endothelial TGFβ signaling polypeptide selected from the group consisting of TGFβ 1, TGFβ2, TGFβ3, TGFβR1, and TGFβR2, thereby reducing or inhibiting the atherosclerotic lesion in the subject.

In another aspect, the invention comprises a method of inhibiting progression of atherosclerosis in a subject, the method comprising administering to the subject an agent that increases the activity or level of let-7 miRNA in an endothelial cell in the subject, thereby inhibiting progression of atherosclerosis in the subject.

In another aspect, the invention comprises a method of inhibiting progression of atherosclerosis in a subject, the method comprising administering to the subject an agent that decreases in an endothelial cell in the subject the activity or level of a TGFβ signaling polypeptide selected from the group consisting of TGFβ1, TGFβ2, TGFβ3, TGFβR1, and TGFβR2, thereby inhibiting progression of atherosclerosis in the subject.

In another aspect, the invention comprises a method of reversing atherosclerosis in a subject, the method comprising administering to the subject an agent that increases the activity or level of let-7 miRNA in an endothelial cell in the subject, thereby reversing atherosclerosis in the subject.

In another aspect, the invention comprises a method of reversing atherosclerosis in a subject, the method comprising administering to the subject an agent that decreases in an endothelial cell in the subject the activity or level of a TGFβ signaling polypeptide selected from the polypeptide selected from the group consisting of TGFβ 1, TGFβ2, TGFβ3, TGFβR1, and TGFβR2, thereby reversing atherosclerosis in the subject.

In another aspect, the invention comprises a method of treating atherosclerosis in a subject, the method comprising administering to the subject an agent that increases the activity or level of let-7 miRNA in an endothelial cell in the subject, thereby treating atherosclerosis in the subject.

In another aspect, the invention comprises a method of treating atherosclerosis in a subject, the method comprising administering to the subject an agent that decreases in an endothelial cell in the subject the activity or level of a TGFβ signaling polypeptide selected from the group consisting of TGFβR1, and TGFβR2, thereby treating atherosclerosis in the subject.

In another aspect, the invention comprises a method of inhibiting progression of atherosclerosis in a subject, the method comprising administering to the subject an agent that decreases the activity or level of FRS2a in a smooth muscle cell in the subject, thereby inhibiting progression of atherosclerosis in the subject.

In another aspect, the invention comprises a method of reversing atherosclerosis in a subject, the method comprising administering to the subject an agent that decreases the activity or level of FRS2α in a smooth muscle cell in the subject, thereby reversing atherosclerosis in the subject.

In another aspect, the invention comprises a method of treating atherosclerosis in a subject, the method comprising administering to the subject an agent that decreases the activity or level of FRS2α in a smooth muscle cell in the subject, thereby treating atherosclerosis in the subject.

In various embodiments, the agent is selectively delivered to an endothelial cell in the subject.

In various embodiments, the agent is in a nanoparticle.

In various embodiments, the nanoparticle is a 7C1 nanoparticle.

In various embodiments, the agent is selectively delivered to a smooth muscle cell in the subject.

In various embodiments, the agent is administered intravenously.

In various embodiments, the agent that increases the activity or level of let-7 miRNA is selected from the group consisting of human let-7b miRNA and human let-7c miRNA.

In various embodiments, the agent that increases the activity or level of let-7 miRNA is one of the above described compositions.

In various embodiments, the agent that decreases the activity or level of a TGFβ signaling polypeptide is an inhibitory polynucleotide that reduces expression of the TGFβ signaling polypeptide.

In various embodiments, the agent that decreases the activity or level of FRS2α is an inhibitory polynucleotide that reduces expression of a FRS2α polypeptide.

In various embodiments, the increased level of let-7 miRNA in the subject decreases expression of a TGFβ signaling polypeptide, thereby decreasing TGFβ signaling in the cell.

In various embodiments, the decrease in the activity or level of the TGFβ signaling polypeptide inhibits an endothelial-to-mesenchymal transition.

In various embodiments, the decrease in the activity or level of the FRS2α polypeptide promotes smooth muscle cell proliferation.

In various embodiments, the subject is identified as having a decreased level of let-7 miRNA or an increased level or activity of a TGFβ signaling polypeptide in a biological sample obtained from the subject relative to a reference. In various embodiments, the biological sample is an endothelial cell.

In various embodiments, the subject is identified as having an increased level of let-7 miRNA or a decreased level or activity of a TGFβ signaling polypeptide in a biological sample obtained from the subject relative to a reference.

In various embodiments, the biological sample is a smooth muscle cell.

In various embodiments, the subject is human.

In another aspect, the invention comprises a method of identifying an agent that modulates atherosclerosis, the method comprising measuring the activity or level of a TGFβ signaling polypeptide or polynucleotide, a let-7 miRNA, or a FGF signaling polypeptide or polynucleotide in a cell contacted with a candidate agent, wherein an alteration in the activity or level of the TGFβ signaling polypeptide or polynucleotide, the let-7 miRNA, or the FGF signaling polypeptide or polynucleotide relative to a reference indicates the candidate agent modulates atherosclerosis. In various embodiments, the TGFβ signaling polypeptide or polynucleotide is a TGFβ1, TGFβ2, TGFβ3, TGFβR1, or a TGFβR2 polypeptide or polynucleotide. In various embodiments, the FGF signaling polypeptide is FRS2a. In various embodiments, the cell is an endothelial cell. In various embodiments, an increase in the activity or level of let-7 miRNA or FGF signaling polypeptide or polynucleotide or a decrease in the activity or level of a TGFβ signaling polypeptide or polynucleotide indicates the candidate agent inhibits progression or reverses atherosclerosis. In various embodiments, the cell is a smooth muscle cell. In various embodiments, a decrease in the activity or level of let-7 miRNA or FGF signaling polypeptide or an increase in the activity or level of a TGFβ signaling polypeptide or polynucleotide indicates the candidate agent inhibits progression or reverses atherosclerosis.

In another aspect, the invention comprises a method of reducing, inhibiting or reversing an endothelial-to-mesenchymal transition (EndMT) in an endothelial cell in a subject in need thereof, the method comprising administering to the subject an agent that decreases in the endothelial cell of the subject the activity or level of at least one selected from the group consisting of let-7 miRNA, endothelial TGFβ signaling polypeptide and FRS2a, thereby reducing, inhibiting or reversing the EndMT in the endothelial cell in the subject in need thereof.

In various embodiments, the TGFβ signaling polypeptide is selected from the group consisting of TGFβ 1, TGFβ2, TGFβ3, TGFβR1, and TGFβR2. In various embodiments, the let-7 miRNA is selected from the group consisting of human let-7b miRNA and human let-7c miRNA.

In various embodiments, the methods further comprise administering to the subject an additional agent comprising a therapeutically effective amount of rapamycin or any derivative thereof. In various embodiments, the agent that decreases the activity or level of a TGFβ signaling polypeptide and the additional agent are co-administered to the subject.

In various embodiments, the agent that decreases the activity or level of a TGFβ signaling polypeptide is a nucleic acid capable of downregulating the gene expression of at least one gene selected from the group selected from the group consisting of TGFβ 1, TGFβ2, TGFβ3, TGFβR1, and TGFβR2. In various embodiments, the at least one gene is selected from the group consisting of TGFβR1, and TGFβR2. In various embodiments, the nucleic acid is selected from the group consisting of an antisense RNA, siRNA, shRNA, and a CRISPR system. In various embodiments, the nucleic acid is combined with a therapeutically effective amount of rapamycin or any derivative thereof. In various embodiments, the nucleic acid is encapsulated in a nanoparticle formulated for selective delivery to an endothelial cell, in a pharmaceutically acceptable excipient. In various embodiments, the nanoparticle is a 7C1 nanoparticle.

In another aspect, the invention comprises a method of reducing, inhibiting or reversing an endothelial-to-mesenchymal transition (EndMT) in an endothelial cell in a subject in need thereof, the method comprising administering to the subject at least one siRNA that decreases in the endothelial cell of the subject the activity or level of at least one TGFβ receptor, thereby reducing, inhibiting or reversing the EndMT in the endothelial cell in the subject in need thereof.

In various embodiments, the at least one TGFβ receptor comprises TGFβR1 or TGFβR2.

### BRIEF DESCRIPTION OF THE DRAWINGS

FIGS. 1A-1C are plots and an immunoblot showing that FRS2α knockdown activates TGFβ signaling in primary human aortic smooth muscle cells (HASMCs). FIGS. 1A-1B show qRT-PCR analysis of TGFβ ligands, TGFβ receptors, and TGFβ target expression in control and FRS2α knockdown HASMCs (NS: not significant compared to control, *p<0.05; **p<0.01; ***p<0.001 compared to control; unpaired two-tailed Student's t test). 3-actin was used for sample loading normalization. Histogram of qRT-PCR results are representative of three independent experiments. FIG. 1C shows an immunoblot analysis of TGFβRs, phosphorylated Smad2 (p-Smad2), and phosphorylated Smad3 (p-Smad3) in control and FRS2α knockdown HASMCs. Blots are representative of four independent experiments.

FIGS. 2A-2F are images and plots showing that FRS2α knockdown increases smooth muscle marker gene expression via the TGFβ pathway in primary human aortic smooth muscle cells (HASMCs). FIG. 2A shows an immunoblot analysis of smooth muscle marker gene expression in control and FRS2α knockdown HASMCs. Blots are representative of four independent experiments. FIG. 2B shows a qRT-PCR analysis of SMC transcription factor gene expression in control and FRS2α knockdown HASMCs (*p<0.05; **p<0.01; ***p<0.001 compared to control; unpaired two-tailed Student's t test. N=3). R-actin was used for sample loading normalization. FIG. 2C shows results of collagen gel contraction assays used to determine the contractile ability of control or FRS2α knockdown HASMCs (*p<0.05 compared to control; unpaired two-tailed Student's t test. N=3). FIGS. 2D-2F show immunoblots of smooth muscle markers, phosphorylated Smad2 (p-Smad2), and TGFβR1 expression in control and FRS2α knockdown HASMCs treated with SB431542 (10 μm), TGFβR2 or Smad2 shRNA lentiviruses. Blots are representative of three independent experiments.

FIGS. 3A-3E are plots and images showing FRS2α knockdown increases smooth muscle marker gene expression via the let-7-TGFβ pathway in primary human aortic smooth muscle cells (HASMCs). FIG. 3A shows a quantitative real-time PCR analysis of mature let-7 family in control and FRS2α knockdown HASMCs. SNORD47 was used to normalize the variability in template loading. Histogram of qRT-PCR results are three independent experiments. FIG. 3B shows immunoblots of SM-calponin, phosphorylated Smad2 (p-Smad2), and TGFβR1 expression in control and FRS2α knockdown HASMCs transduced with let-7b lentiviruses. Blots are representative of three independent experiments. FIG. 3C shows phase-contrast and immunofluorescence staining of smooth muscle markers in HASMCs. Nuclei were counterstained with DAPI. Scale bar: 12 μm. Images are representative of three independent experiments. FIG. 3D shows quantitative real-time PCR analysis of mature let-7 family in HASMCs. HASMCs were cultured in the growth medium (M231+SMGS) at day 0 then switched from growth conditions to differentiation medium (M231+SMDS) for 8 days. SNORD47 was used to normalize the variability in template loading. Histogram of qRT-PCR results are three independent experiments. FIG. 3E shows immunoblots of smooth muscle markers, phosphorylated Smad2 (p-Smad2), and TGFβR1 expression in control and FRS2α knockdown HASMCs with or without let-7b lentiviruses. Control and FRS2α knockdown HASMCs were cultured in the growth medium (M231+SMGS) at day 0 then switched from growth conditions to differentiation medium (M231+SMDS) for 6 days with or without let-7b lentiviruses. Blots are representative of three independent experiments.

FIGS. 4A-4H are plots and images showing FGFR1 signaling activity in smooth muscle cells in human left main coronary arteries with various degrees of atherosclerosis. FIG. 4A shows coronary arteries dissected from the human heart. Left main (LM), left anterior descending (LAD), and left circumflex (LCX) branches Scale bar: 1 cm. FIG. 4B shows Elastic-Van Gieson (EVG) staining of human coronary arteries demonstrating various degrees of atherosclerosis. FIGS. 4C-4D are representative images of immunofluorescence staining for CD31 and SM α-actin or SM-MHC in No/mild, moderate, and severe disease human left main coronary arteries. No: no-disease. Nuclei were stained with DAPI. Images are representative of ten No/mild, nine moderate and ten severe disease human left main coronary artery samples. Scale bar: 16 μm. FIGS. 4E and 4G show representative images of immunofluorescence staining for p-FGFR1 or FGFR1 in the same patient cohort. Nuclei were counter-stained with DAPI. Scale bar: 16 μm. FIGS. 4F and 4H show percentage of medial p-FGFR1⁺ SMC and FGFR1⁺ SMC (***p<0.001 compared to No/mild disease, NS: not significant compared to No/mild disease; one-way ANOVA with Newman-Keuls post hoc test for multiple comparison correction).

FIGS. 5A-5F are plots and images showing TGFβ signaling activity in smooth muscle cells in human left main coronary arteries with various degrees of atherosclerosis. FIGS. 5A, 5C, and 5E show representative images of immunofluorescence staining for TGFβ, p-Smad2, or p-Smad3 from patients with No/mild, moderate, or severe disease. Nuclei were counter-stained with DAPI. Scale bar: 16 μm. FIGS. 5B, 5D, and 5F show percentage of medial TGFβ, p-Smad2, and p-Smad3 (***p<0.001 compared to No/mild disease; one-way ANOVA with Newman-Keuls post hoc test for multiple comparison correction).

FIGS. 6A-6J are plots and images showing FGFR1 and TGFβ signaling activity in smooth muscle cells in a mouse atherosclerosis model. FIG. 6A shows a dissected mouse aorta demonstrating lipid-rich plaques in brachiocephalic artery after 4 months of high fat diet compared to the normal diet in Apoe^{-/-} mice. Portions of FIG. 6A labeled “b” and “d,” respectively show a cross-section of brachiocephalic artery from the portions of FIG. 6A labeled “a” and “c” stained with Oil Red O. FIG. 6B shows a histological analysis of mouse normal artery or atherosclerotic plaque in brachiocephalic artery with anti-SM α-actin, anti-Notch3, and anti-SM-MHC antibodies. Nuclei were counterstained with DAPI. Scale bar: 62 μm. FIGS. 6C-6F shows analysis of brachiocephalic artery of Apoe^{-/-} mice maintained for 4 months on either normal or high fat diet using anti-CD31, anti-p-FGFR1, anti-FGFR1, anti-p-Smad2, and anti-p-Smad3 antibodies. Nuclei counterstained with DAPI. Scale bar: 62 μm. (6 mice/group). L: lumen. M: Media. FIGS. 6G-6J show quantification of the number of media smooth muscle cells expressing p-FGFR1, FGFR1, p-Smad2, and p-Smad3 (***p<0.001 compared to ND, NS: not significant compared to ND; unpaired two-tailed Student's t test.). ND: Normal diet. HFD: High fat diet.

FIGS. 7A-7H are plots and images showing smooth muscle cell FRS2α knockout inhibits atherosclerosis plaque development after 16 weeks of high fat diet. FIG. 7A shows representative photomicrographs of Oil Red O-stained atherosclerotic lesions in the aortic arch, of Apoe^{-/-} or Frs2^{SMCKO}/Apoe^{-/-} mice after 16 weeks of high fat diet. FIG. 7B (on the left) shows microphotographs of aortas (en face) from Apoe^{-/-} and Frs2^{SMCKO}/Apoe^{-/-} mice after 16 weeks of high fat diet after staining with Oil Red O. Shown on the right of FIG. 7B is lesion area quantification. All data shown as mean±SD. (***p<0.001 compared to Apoe^{-/-}; unpaired two-tailed Student's t test). FIGS. 7C-7D show representative cross-sections of brachiocephalic arteries Apoe^{-/-} and Frs2^{SMCKO}/Apoe^{-/-} mice stained with hematoxylin and eosin (H&E) (C) and Movat (D). (a)&(b) are high magnification view of the atherosclerotic plaque shown by black dot boxes. NC: necrotic core. FIG. 7E shows histological analysis of atherosclerotic plaque with anti-Ki67 antibody. Nuclei were counterstained with DAPI. Scale bar: 62 μM. FIG. 7F shows quantification of plaque cellularity; Apoe^{-/-} mice N=9, Frs2^{SMCKO}/Apoe^{-/-} mice N=12 (***p<0.001 compared to Apoe^{-/-}; unpaired two-tailed Student's t test). FIG. 7G shows quantifications of the extent of fibrous cap and necrotic areas in brachiocephalic artery of Apoe^{-/-} and Frs2^{SMCKO}/Apoe^{-/-} mice. Apoe^{-/-} mice N=9, Frs2^{SMCKO}/Apoe^{-/-} mice N=12. (*p<0.05, **p<0.01 compared to Apoe^{-/-}; unpaired two-tailed Student's t test). FIG. 7H shows measurement of Ki67⁺ cells (*p<0.05, ***p<0.001 compared to Apoe^{-/-}; unpaired two-tailed Studert's t test).

FIG. 8 is a schematic showing a scheme of FGF-dependent regulation of TGFβ signaling in smooth muscle cells and endothelial cells. In both smooth muscle cells and endothelial cells, suppression of FGF signaling leads to reduction of let-7 miRNAs expression that, in turn, results in increased TGFβR1 expression and activation of TGFβ-dependent transcriptional program. In SMC (left panel), activation of TGFβ signaling promotes SMC conversion from proliferative to contractile phenotype thereby reducing the number of SMCs in the plaque and reducing plaque growth. In contrast, in endothelial cells (EC) activation of TGFβ signaling promotes endothelial-to-mesenchymal transition thus increasing the number of plaque SMCs and promoting plaque growth.

FIGS. 9A-9C are plots and images showing that FRS2α knockdown inhibits proliferation of human aortic smooth muscle cells (HASMCs). FIG. 9A shows control and FRS2α knockdown HASMCs that were cultured in the growth medium (M231+SMGS). Cell proliferation was analyzed using real-time cell analysis (xCELLigence). Cell proliferation curves are representative of three independent experiments (***p<0.05 compared to control; unpaired two-tailed Student's t test). FIG. 9B shows control and FRS2α knockdown HASMCs were cultured in the growth medium (M231+SMGS). Immunoblots of cell cycle regulators Cyclin D1, p21, and p27 in control and FRS2α knockdown HASMCs. Blots are representative of four independent experiments. FIG. 9C shows control and FRS2α knockdown HASMCs cultured in the growth medium (M231+SMGS). Flow cytometry analysis with propidium iodide (PI) staining was used to evaluate the percentage of cellular DNA content in control and FRS2α knockdown HASMCs. Histogram of cell cycle distribution results are representative of three independent experiments.

FIGS. 10A-10E are plots and images showing FGFR1 knockdown activates TGFβ signaling and induces smooth muscle marker gene expression in primary human aortic smooth muscle cells (HASMCs). FIG. 10A shows qRT-PCR analysis of FGFRs, FRS2α, and Klotho family gene expression in primary human aortic smooth muscle cells (HASMCs). Data are presented as mean±SD. 3-actin was used for sample loading normalization. Histogram of qRT-PCR results are representative of four independent experiments. FIGS. 10B-10C show qRT-PCR analysis of TGFβ ligands, TGFβ receptors, and downstream target genes in control and FGFR1 knockdown HASMCs. (NS: not significant compared to control, *p<0.05; **p<0.01; ***p<0.001 compared to control; unpaired two-tailed Student's t test). β-actin was used for sample loading normalization. Histogram of qRT-PCR results are representative of three independent experiments. FIG. 10D shows qRT-PCR analysis of smooth muscle cell transcription factors and smooth muscle marker gene expression in control and FGFR1 knockdown HASMCs. (*p<0.05; **p<0.01; ***p<0.001 compared to control; unpaired two-tailed Student's t test. N=3). 3-actin was used for sample loading normalization. FIG. 10E shows an immunoblot analysis of TGFβ signaling, TGFβ downstream targets, and smooth muscle markers in control and FGFR1 knockdown HASMCs. Blots are representative of four independent experiments.

FIGS. 11A-11H are plots and images showing Frs2α^{SMCKO} mice display normal vascular morphology and vascular density. FIG. 11A shows a qRT-PCR analysis of Frs2α expression in mouse aorta (***p<0.001 compared to control). 3-actin was used for sample loading normalization. All of the data represent the mean±SD. 3 control and 3 Frs2αα^{SMCKO} mice were analyzed. FIG. 11B shows an immunoblot analysis of FRS2α expression in mouse aorta. In each group aorta were pooled from 4 mice/group. FIG. 11C shows representative images of FRS2α immunofluorescence staining of control and Frs2αα^{SMCKO} aorta. Endothelial cells are visualized by CD31. Black arrows indicate endothelial cells. L: lumen. Nuclei were stained with DAPI. Images are representative of 3 mice/group. Scale bar: 10 μm. FIG. 11D shows gross appearance of aorta in 8-week-old control and Frs2αα^{SMCKO} mice. Asc: Ascending; Desc: Descending. FIG. 11E shows 5 μm cross-sections of control and Frs2α^{SMCKO} mouse brachiocephalic artery were stained with EVG (elastic Van Gieson), anti-SM α-actin, anti-SM22a, and anti-Notch3 antibodies. Nuclei were counterstained with DAPI. L: lumen. Scale bar: 10 μm. Images are representative of 3 mice/group. FIG. 11F (left) shows a histological analysis of control and Frs2α^{SMCKO} mouse brachiocephalic artery with anti-CD31 and anti-p-Smad2 antibodies. Nuclei were counterstained with DAPI. L: lumen. Scale bar: 10 μm. Right: Percentage of p-Smad2⁺ cells in the media (NS: not significant compared to control; unpaired two-tailed Student's t test). Images are representative of 6 mice/group. FIGS. 11G-11H (left) show representative images of vascular structure in heart and skeletal muscle in control and Frs2α^{SMCKO} mice. Scale bar: 62 μm for 100× and 16 μm for 400×. Right: Vascular density was quantified (NS: not significant compared to control; unpaired two-tailed Student's t test). Images are representative of 5 mice/group.

FIGS. 12A-12D are plots and images showing Frs2α^{SMCKO} mice have normal body weight, lipid profiles, and heart function. FIGS. 12A-12B show body weight, total cholesterol, triglycerides, and HDL-C analysis of Apoe^{-/-} and Frs2a^{SMCKO}/Apoe^{-/-} mice before and after 16 weeks on a high cholesterol diet. (NS: not significant compared to Apoe^{-/-}; unpaired two-tailed Student's (test). FIG. 12C shows representative ultrasound images and ascending aorta diameters of Apoe^{-/-} and Frs2α^{SMCKO}/Apoe^{-/-} mice. All of the data represent the mean±SD. (NS: not significant compared to Apoe^{-/-}; unpaired two-tailed Student's t test). 3 Apoe^{-/-} and 3 Frs2α^{SMCKO}/Apoe^{-/-} mice were analyzed. FIG. 12D shows an echocardiographic analysis in Apoe^{-/-} and Frs2α^{SMCKO}/Apoe^{-/-} mice showed no effect on cardiac output, ejection fraction, and fractional shortening. All of the data represent the mean±SD. (NS: not significant compared to Apoe^{-/-}; unpaired two-tailed Student's t test). 3 Apoe^{-/-} and 3 Frs2α^{SMCKO}/Apoe^{-/-} mice were analyzed.

FIGS. 13A-13D are plots and images showing that smooth muscle cell FRS2α knockout inhibits atherosclerosis plaque development. FIG. 13A shows representative photomicrographs of Oil Red O-stained atherosclerotic lesions in the aortic arch, of Apoe^{-/-} or Frs2^{SMCKO}/Apoe^{-/-} mice after 2 months of high fat diet or normal diet. FIG. 13B (left) shows microphotographs of aortas (en face) from Apoe^{-/-} and Frs2^{SMCKO}/Apoe^{-/-} mice after 2 months of high fat diet after staining with Oil Red 0; (right) lesion area quantification. All data shown as mean±SD. (**p<0.01 compared to Apoe^{-/-}; unpaired two-tailed Student's t test).

FIG. 13C shows quantification of SM α-actin area in the plaque from Apoe^{-/-} and Frs2^{SMCKO}/Apoe^{-/-} mice after 4 months of high fat diet. Apoe^{-/-} mice N=9, Frs2^{SMCKO}/Apoe^{-/-} mice N=12 (*p<0.05 compared to Apoe^{-/-}; unpaired two-tailed Student's t test). Nuclei were counterstained with DAPI. Scale bar: 62 μm. FIG. 13D shows measurement of Collagen 1 area from Apoe^{-/-} and Frs2^{SMCKO}/Apoe^{-/-} mice after 4 months of high fat diet (*p<0.05 compared to Apoe^{-/-}; unpaired two-tailed Student's t test). Data expressed as the ratio of collagen 1 signal to the total vessel area. Apoe^{-/-} mice N=9, Frs2^{SMCKO}/Apoe^{-/-} mice N=12. Nuclei were counterstained with DAPI. Scale bar: 62 μm.

FIGS. 14A-14C are immunoblots showing TGFβ signaling in TGFβR1, TGFβR2, and TGFβR1/2 knockdown backgrounds. Each of FIGS. 14A-14C shows levels of TGFβR1, TGFβR2, and TGFβR3, and levels of p-Smad2 (phosphorylated Smad2), Smad-2, p-Smad3 (phosphorylated Smad3), and Smad 2/3 in a TGFβR1, TGFβR2, and TGFβR1/2 knockdown background, respectively.

FIGS. 15A-15C are immunoblots showing BMP signaling in TGFβR1, TGFβR2, and TGFβR1/2 knockdown backgrounds. Each of FIGS. 15A-15C shows levels of p-Smad1/5/8 (phosphorylated Smad1/5/8), Smad-5, activin receptor-like kinase 1 (ALK1), bone morphogenetic protein receptor (BMPR2), endoglin, TGFβR1, and TGFβR2 in a TGFβR1, TGFβR2, and TGFβR1/2 knockdown background, respectively.

FIGS. 16A-16D are schematics and blots depicting the generation and characterization of Apoe^{-/-} mice with endothelial-specific Tgfbr1 and Tgfbr2 ablation. FIG. 16A: Scheme of the Cdh5-CreER^{T2} transgene, Tgfbr1, Tgfbr2 floxed alleles, and R26-mTmG reporter constructs. FIG. 16B: PCR analysis using tail genomic DNA of the indicated genotypes, FIG. 16C shows a setup of experiments investigating TGFβ signaling and atherosclerotic plaque development using the Tgfbr^{iECKO}/Apoe^{-/-} mice, as described elsewhere herein. FIG. 16D are immunoblots showing TGFβ (upper) and BMP (bottom) signaling in Tgfbr^{iECKO}/Apoe^{-/-} mouse endothelial cells. Heart endothelial cells were isolated from vehicles or tamoxifen treated mice and were treated with TGFβ 1 (0.5 ng/ml, upper) or BMP9 (0.5 ng/ml, bottom) for the indicated times and downstream signaling was analyzed by immunoblotting. In each group, endothelial cells were isolated and pooled from 3 mice/group.

FIGS. 17A-17D are series of graphs demonstrating that endothelial cell Tgfbr1/Tgfbr2 knockout have no effect on body weight and serum lipid profile. FIG. 17A: Scheme of Tamoxifen injection (1 mg/day i.p. for 5 days starting at 6 week old) and high fat diet (HFD) feeding. FIG. 17B: Body weight analysis of Apoe^{-/-} and Tgfbr^{iECKO}/Apoe^{-/-} mice before and after 4, 8, 12, 16 weeks on a high cholesterol diet. (NS: not significant compared to Apoe^{-/-}; unpaired two-tailed Student's t test). FIGS. 17C-17D: Serum total cholesterol and triglycerides levels from Apoe^{-/-} and Tgfbr^{iECKO}/Apoe^{-/-} mice before and after 16 weeks on a high cholesterol diet. (NS: not significant compared to Apoe^{-/-}; unpaired two-tailed Student's t test).

FIG. 18 is an image showing no plaque development in mice fed with a normal diet at 8 weeks old.

FIG. 19 is an image showing 61% plaque reduction in Tgfbr^{iECKO}/Apoe^{-/-} mice after 1 month on a high fat diet (HFD).

FIG. 20 is set of images and a plot showing 72% plaque reduction in Tgfbr^{iECKO}/Apoe^{-/-} mice after 2 months on a high fat diet (HFD).

FIG. 21 is a set of images and a plot showing 52% plaque reduction in Tgfbr^{iECKO}/Apoe^{-/-} mice after 3 months on a high fat diet (HFD).

FIGS. 22A-22F are a set of images demonstrating that endothelial cell Tgfbr1/Tgfbr2 knockout inhibits atherosclerosis plaque development. (FIG. 22A) (Left) Microphotographs of aortas (en face) from Apoe^{-/-} or Tgfbr^{iECKO}/Apoe^{-/-} mice after 0, 1, 2, 3, 4 months of high fat diet staining with Oil Red O. (Right) Lesion area quantification. % Lesion area is lesion area/total area of aorta. All data shown as mean±SD. (***p<0.001 compared with Apoe^{-/-}; unpaired two-tailed Student's t test). 3-11 mice per group. (FIG. 22B) Representative photomicrographs of Oil Red O-stained atherosclerotic lesions in the aortic arch of Apoe^{-/-} or Tgfbr^{iECKO}/Apoe^{-/-} mice after 0, 1, 2, 3, 4 months of high fat diet. 3 mice per group. Scale bar: 5 mm. (FIG. 22C) (Left) Representative examples of cross-sections from the aortic root after 4 months of high fat diet stained with Oil Red O. Scale bar: 200 μm. 11 mice/group. (Right) Quantification of aortic root lesion areas. Mean±SD. (***p<0.001 compared to Apoe^{-/-}; unpaired two-ailed Student's t test). (FIG. 22D) Representative images of brachiocephalic artery from Apoe^{-/-} and Tgfbr^{iECKO}/Apoe^{-/-} mice stained with Movat. Scale bar: 100 μm. (FIG. 22E) Measurement of lesion area (***p<0.001 compared to Apoe^{-/-}; unpaired two-tailed Student's t test). (FIG. 22F) Quantifications of the extent of necrotic areas in brachiocephalic artery of Apoe^{-/-} and Tgfbr^{iECKO}/Apoe^{-/-} mice (***p<0.001 compared to Apoe^{-/-}; unpaired two-tailed Student's (test).

FIG. 23 is a set of images showing Apoe^{-/-} and Frs2α^{iECKO}/Apoe^{-/-} mice injected with let-7 miRNA (let-7 heavy (mi-let-7b_{H}); let-7 light (mi-let-7b_{L})) or a luciferase control.

FIGS. 24A-24E are a series of graphs showing that 7C1-let-7 mimics treatment have no effect on mouse body weight and serum lipid profile. FIG. 24A: Time frame of gene inactivation and 7C1 lipid nanoparticle injections. FIG. 24B: Serum triglycerides, total cholesterol, and HDL-C levels from Apoe^{-/-} and Frs2^{iECKO}/Apoe^{-/-} mice after 16 weeks on a high cholesterol diet (NS: not significant by one-way ANOVA with Newman-Keuls post-hoc test). FIG. 24C: Individual body weights in each group were measured every week. FIGS. 24D-24E: qPCR analysis of let-7b and Tgfbr1 expression in lung endothelial cells after treatment with 7C1-let-7 particles. All data shown as mean±s.d. (*p<0.05; **p<0.01; ***p<0.001 compared with Luciferase; one-way ANOVA with Newman-Keuls post hoc test for multiple comparison correction). N=4-6 mice per group.

FIG. 25 is a set of plots showing blood cell counts (white blood cell (WBC), red blood cell (RBC), platelet (PLT), lymphocyte, and monocyte counts) in Apoe^{-/-} and Frs2α^{iECKO}/Apoe^{-/-} mice.

FIG. 26 is a set of images showing organs harvested from Apoe^{-/-} and Frs2α^{iECKO}/Apoe^{-/-} mice injected with let-7 miRNA (let-7 heavy, let-7 light) or a luciferase control.

FIG. 27 is a set of plots showing weight of organs (heart, lung, liver, kidney, spleen) harvested from Apoe^{-/-} and Frs2α^{iECKO}/Apoe^{-/-} mice injected with let-7 miRNA (let-7 heavy, let-7 light) or a luciferase control.

FIG. 28 is set of plots showing results of liver function test in Apoe^{-/-} and Frs2α^{iECKO}/Apoe^{-/-} mice injected with let-7 miRNA (let-7 heavy, let-7 light) or a luciferase control.

FIG. 29 is a set of images and plot showing Oil Red-O staining of whole aorta obtained from Apoe^{-/-} and Frs2α^{iECKO}/Apoe^{-/-} mice injected with let-7 miRNA (let-7 heavy, let-7 light) or a luciferase control.

FIG. 30 is a set of plots showing triglyceride, cholesterol, and high density lipoprotein (HDL) levels in Apoe^{-/-} and Frs2α^{iECKO}/Apoe^{-/-} mice injected with let-7 miRNA (let-7 heavy, let-7 light) or a luciferase control.

FIG. 31 is a set of plots showing let-7 and target gene expression in isolated lung endothelial cells in Apoe^{-/-} and Frs2α^{iECKO}/Apoe^{-/-} mice injected with let-7 miRNA (let-7 heavy, let-7 light) or a luciferase control.

FIGS. 32A-32D are plots and blots showing TGFβ and BMP signaling in a FRS2α knockdown background. FIG. 32A shows levels of Type 1, Type II, and Type III TGFβ receptors in a FRS2α knockdown background. FIG. 32B shows levels of TGFβ and BMP signaling components in a FRS2α knockdown background. FIG. 32C shows a time course of levels of TGFβ signaling components in a FRS2α knockdown background. FIG. 32D shows a time course of levels of BMP signaling components in a FRS2α knockdown background.

FIGS. 33A-33B are blots showing TGFβ and BMP signaling, respectively, in a ALK1 knockdown, TGFβR2 knockdown, FRS2α knockdown, ALK1/FRS2α knockdown, and TGFβR2/FRS2α knockdown background.

FIGS. 34A-34C are blots and an image showing MAPK signaling in a FRS2α knockdown background. FIGS. 34A and 34C show levels of MAPK signaling components in a FRS2α knockdown. FIG. 34B shows an analysis using anti-VE cadherin and anti-active 3-catenin. Nuclei were counterstained with DAPI.

FIGS. 35A-35C are images showing TGFβ signaling activity in endothelial cells from subjects having No/mild disease, moderate disease, and severe disease, using anti-CD31, anti-p-Smad3, and anti-p-Smad5 antibodies. FIG. 35A shows immunostaining for p-Smad3.

FIG. 35B shows immunostaining for p-Smad5. FIG. 35C shows quantification of immunocytochemistry data from FIG. 35B. Nuclei were counterstained with DAPI.

FIG. 36 are images showing TGFβ signaling activity in arteries from subjects having No/mild disease, moderate disease, and severe disease, using anti-CD31 and anti-TGFβ antibodies. Nuclei were counterstained with DAPI.

FIGS. 37A-37B are images and a plots showing NKX2.5 expression in endothelial cells from subjects having No/mild disease, moderate disease, and severe disease. Nuclei were counterstained with DAPI. FIG. 37A shows immunostaining for NKX2.5. FIG. 37B shows quantification of immunocytochemistry data from FIG. 37A.

FIGS. 38A-38D are a series of images and histograms depicting that 7C1-let-7 mimics suppress atherosclerosis lesion development in both Apoe^{-/-} and Frs2α^{iECKO}/Apoe^{-/-} mice. Mice were injected intravenously with PBS, 7C1-Luciferase, and 7C1-let-7 mimics and concomitantly fed the high fat diet for 4 months (n=4 to 6 per group). FIGS. 38A-38B (Left) Representative images of the Oil Red O-stained atherosclerotic lesions in the aorta from Apoe^{-/-} or Frs2α^{iECKO}/Apoe^{-/-} mice treated with PBS, Luciferase, or let-7 mimics. (Right) Lesion area quantification. All data shown as mean±s.d. (***p<0.001 compared with Luciferase treated group; one-way ANOVA with Newman-Keuls post hoc test for multiple comparison correction). FIGS. 38C-38D (Upper) Representative images of brachiocephalic artery from PBS, Luciferase, or let-7 mimics treated mice stained with Movat (scale bar: 200 μm). (Bottom) Quantifications of the lesion area and the extent of necrotic core areas in brachiocephalic artery of PBS, Luciferase, or let-7 mimics treated mice (*p<0.05; ***p<0.001 compared with Luciferase; one-way ANOVA with Newman-Keuls post hoc test for multiple comparison correction).

FIGS. 39A-39H are a series of images and histograms depicting that endothelial cell Tgfbr1/Tgfbr2 knockout facilitates regression of advanced murine atherosclerotic plaques. FIG. 39A: Diet and treatment schemes. After 2 months of high-fat-diet, the mice were treated with tamoxifen or vehicle control. Then the high-fat-diet was continued for another 2 months. FIG. 39B: (Left) Representative images of the Oil Red O-stained atherosclerotic lesions in the aorta from Apoe^{-/-} or Tgfbr^{iECKO}/Apoe^{-/-} mice. (Right) Lesion area quantification. All data shown as mean±s.d. (NS: not significant; ***p<0.001; one-way ANOVA with Newman-Keuls post hoc test for multiple comparison correction). FIG. 39C: Diet and treatment schemes. Mice were fed the high-fat-diet for 3 months to induce advanced atherosclerotic lesions. Then the diet was changed to a normal diet for another month. Mice were simultaneously treated with tamoxifen or vehicle control. FIG. 39D: Representative images of the Oil Red O-stained atherosclerotic lesions in the aorta from Apoe^{-/-} or Tgfbr^{iECKO}/Apoe^{-/-} mice. FIG. 39E: Diet and treatment schemes. Mice were fed the high-fat-diet for 3 months to induce advanced atherosclerotic lesions. Then the diet was either changed to a normal diet for another 1 or 2 months. Mice were simultaneously treated with tamoxifen or vehicle control. FIG. 39F: Representative images of the cross-sections from the aortic root after 4 months of high fat diet stained with Movat (scale bar: 200 μm). FIG. 39G: Lesion area quantification shown in FIG. 39D. All data shown as mean±s.d. (**p<0.01; unpaired two-tailed Student's t test). FIG. 39H: Aortic root lesion area quantification shown in FIG. 39F. All data shown as mean±s.d. (***p<0.001; one-way ANOVA with Newman-Keuls post hoc test for multiple comparison correction).

FIGS. 40A-40B are a series of images and histograms depicting that endothelial cell Tgfbr1/Tgfbr2 knockout reduce plaque cellularity and inhibit SM α-actin, collagen 1, fibronectin, and VCAM-1 expression in the plaques. FIG. 40A: Histological analysis of atherosclerotic plaques from Apoe^{-/-} and Tgfbr^{iECKO}/Apoe^{-/-} mice stained with Hematoxylin and eosin (H&E) and anti-SM α-actin, anti-collagen 1, anti-fibronectin, and anti-VCAM1 antibodies. Nuclei were counterstained with DAPI. Scale bar: 62 μm. FIG. 40B: Measurement of plaque cell number, SM α-actin, collagen 1, fibronectin, and VCAM-1 area (*p<0.05; **p<0.01; ***p<0.001 compared to Apoe^{-/-}; one-way ANOVA with Newman-Keuls post hoc test for multiple comparison correction).

FIGS. 41A-41F are a series of images and histograms showing that endothelial Tgfbr1/Tgfbr2 knockout represses EC activation. FIGS. 41A-41D: Heart endothelial cells were isolated from vehicles or tamoxifen treated mice and were treated with TNF-α (10 ng/ml), IL-1β (10 ng/ml), IL-6 (10 ng/ml), or IFN-γ (10 ng/ml) for the indicated times and downstream signaling was analyzed by immunoblotting. In each group, endothelial cells were isolated and pooled from 3 mice/group. FIG. 41E: Histological analysis of thoracic aorta from Apoe^{-/-} and Tgfbr^{iECKO}/Apoe^{-/-} mice injected with either PBS or 100 mg LPS stained for ICAM-1 and VCAM-1. Nuclei were counterstained with DAPI. Scale bar: 62 μm. FIG. 41F: Measurement of ICAM-1 and VCAM-1 area (NS: not significant; **p<0.05; ***p<0.001 compared to Apoe^{-/-}; unpaired two-tailed Student's t test).

FIGS. 42A-42D are a series of images and histograms demonstrating that 7C1-let-7 mimics treatment reduce plaque cellularity, inhibit SM α-expression, macrophage recruitment in the plaques in both Apoe^{-/-} and Frs2α^{iECKO}/Apoe^{-/-} mice. FIG. 42A and FIG. 42C: Histological analysis of atherosclerotic plaques from PBS, 7C1-Luciferase, and 7C1-let-7 mimics treated mice stained with Hematoxylin and eosin (H&E) and anti-SM α-actin, and anti-F4/80 antibodies. Nuclei were counterstained with DAPI. Scale bar: 62 μm. FIG. 42B and FIG. 42D: Measurement of plaque cell number, SM α-actin, F4/80 area (**p<0.01; ***p<0.001 compared to Apoe^{-/-}; one-way ANOVA with Newman-Keuls post hoc test for multiple comparison correction).

FIGS. 43A-43B are a series of images and histograms showing the effects of endothelial cell Tgfbr1/Tgfbr2 knockout on the regression of atherosclerosis macrophage content. Mice were fed the high-fat-diet for 3 months to induce advanced atherosclerotic lesions. Then the diet was changed to a normal diet for additional 1 or 2 months. Mice were simultaneously treated with tamoxifen or vehicle control. FIG. 43A: Histological analysis of aortic root from Apoe^{-/-} and Tgfbr^{iECKO}/Apoe^{-/-} mice stained with anti-F4/80 antibody. Nuclei were counterstained with DAPI. Scale bar: 62 μm. FIG. 43B: Measurement of F4/80 area (NS: not significant; *p<0.05; ***p<0.001; one-way ANOVA with Newman-Keuis pos, hoc test for multiple comparison correction).

FIGS. 44A-44B are a series of histograms depicting the in vivo assessment of siTgfbr1 and siTgfbr2 in heart and lung endothelial cells (EC). FIG. 44A (Lung EC) and FIG. 44B (Heart EC): C57BL/6J mice were injected intravenously with PBS or 7C1-siTgfbr1/Tgfbr2 at different concentrations. Forty-eight hours later, heart and lung endothelial cells were harvested. Expression of Tgfbr1 and Tgfbr2 were analyzed by quantitative real-time PCR. 3-actin was used to normalized the variability in template loading. All data shown as mean±SD. (NS: not significant; *p<0.05; **p<0.01; ***p<0.001 compared with PBS; unpaired two-tailed Student's t test. N=3 mice per group.

FIGS. 45A-45B are a graph and a series of images showing that 7C1-siTgfbr and rapamycin suppress atherosclerosis lesion development in Apoe^{-/-} mice after 4 months of high fat diet. FIG. 45A: Time frame of 7C1-siTgfbr1/Tgfbr2 lipid nanoparticle and rapamycin injections. FIG. 45B: Representative photomicrographs of Oil Red O-stained atherosclerotic lesions in PBS, 7C1-siLuciferase, DMSO, Rapamycin, or 7C1-siTgfbr treated mice.

FIG. 46 is a histogram illustrating the quantification of atherosclerotic lesions from FIG. 45B. Lesion area quantification. % Lesion area is the lesion area/total area of aorta. All data shown as mean±SD. (***p<0.001; one-way ANOVA with Newman-Keuls post hoc test for multiple comparison correction).

### DETAILED DESCRIPTION OF THE INVENTION

The invention features compositions and methods that are useful for treating atherosclerosis in a subject. The invention is based, at least in part, on the discovery of a key molecular mechanism responsible for atherosclerosis progression. The molecular mechanism is based on the relationship between fibroblast growth factor (FGF) signaling, let-7 miRNA expression, and transforming growth factor β (TGFβ) signaling, which contribute to growth of atherosclerotic plaque. Genetic evidence obtained herein confirm that blocking the mechanism responsible for atherosclerosis progression (e.g., activation of endothelial TGFβ signaling) not only prevents atherosclerotic plaque growth but stops its progression and facilitates it regression.

In endothelial cells (EC) and smooth muscle cells (SMC), FGF-signaling induces let-7 miRNA expression, which leads to downregulation of TGFβ signaling. Studies described herein demonstrate that overexpression of let-7 miRNA or a let-7 miRNA mimic in endothelial cells, which downregulated TGFβ signaling, reduced atherosclerotic lesions in mice. Studies herein also demonstrate that disruption of FGF signaling in smooth muscle cells, which reduced let-7 miRNA expression and led to upregulation of TGFβ signaling, reduced atherosclerotic lesions in mice.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

By “agent” is meant any small molecule chemical compound, antibody, nucleic acid molecule, or polypeptide, or fragments thereof. In some embodiments, the agent is a nucleic acid molecule.

By “alteration” is meant a change (increase or decrease) in the expression levels or activity of a gene or polypeptide as detected by standard art known methods such as those described herein. In some embodiments, an alteration in expression level includes a 10% change in expression levels, a 25% change, a 40% change, and a 50% or greater change in expression levels.

“Biological sample” as used herein means a biological material isolated from a subject, including any tissue, cell, fluid, or other material obtained or derived from the subject. In some embodiments, the subject is human. The biological sample may contain any biological material suitable for detecting the desired analytes, and may comprise cellular and/or non-cellular material obtained from the subject. In certain embodiments, the biological sample is an endothelial cell. Biological samples include tissue samples (e.g., cell samples, biopsy samples), such as tissue from the heart or aorta. Biological samples also include bodily fluids, including, but not limited to, blood, blood serum, plasma, saliva, and urine.

By “capture reagent” is meant a reagent that specifically binds a nucleic acid molecule or polypeptide to select or isolate the nucleic acid molecule or polypeptide. In some embodiments, the capture reagent is a probe or primer that specifically binds a polynucleotide encoding a TGFβ signaling polypeptide, a let-7 miRNA, or a FGF signaling polypeptide.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected. In some embodiments, a level of a let-7 miRNA, a TGFβ signaling polypeptide or polynucleotide, or a FGF signaling polypeptide or polynucleotide is detected.

By “disease” is meant any condition or disorder that damages or interferes with the normal function of a cell, tissue, or organ. Examples of diseases include atherosclerosis, pulmonary hypertension, and chronic inflammation induced fibrosis.

By “effective amount” is meant the amount of a required to ameliorate the symptoms of a disease relative to an untreated patient. In particular embodiments, the disease is atherosclerosis. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, an effective amount of an agent that modulates activity or level of a FGF signaling polypeptide, let-7 miRNA, or TGFβ signaling polypeptide is an amount of the agent that reduces the growth and/or formation of atherosclerotic lesions or reverses atherosclerosis in a subject.

As used herein, a “FGF signaling polypeptide” is meant a member or component of a fibroblast growth factor (FGF) signaling pathway. In some embodiments, the FGF signaling polypeptide is FGFR1 polypeptide or FRS2α polypeptide.

By “FGFR1 polypeptide” is meant a polypeptide or fragment thereof having at least about 85% amino acid identity to GenBank Accession No. AAH15035.1 and having a biological activity of a FGFR1 polypeptide. Biological activities of a FGFR1 polypeptide include cell surface receptor activity and tyrosine kinase activity. The sequence at GenBank Accession No. AAH15035.1 is shown below (SEQ ID No: 21):

| | |
|---|---|
| 1 | mwswkcllfw avlvtatlct arpsptlpeq aqpwgapvev esflvhpgdl lqlrcrlrdd |
| 61 | vqsinwlrdg vqlaesnrtr itgeevevqd svpadsglya cvtsspsgsd ttyfsvnvsd |
| 121 | alpssedddd dddssseeke tdntkpnrmp vapywtspek mekklhavpa aktvkfkeps |
| 181 | sgtpnptlrw lkngkefkpd hriggykvry atwsiimdsv vpsdkgnytc iveneygsin |
| 241 | htyqldvver sphrpilqag ipanktvalg snvefmckvy sdpqphiqwl khievngski |
| 301 | gpdnlpyvqi lktagvnttd kemevlhlrn vsfedageyt clagnsigls hhsawltvle |
| 361 | aleerpavmt splyleiiiy ctgafliscm vgsvivykmk sgtkksdfhs qmavhklaks |
| 421 | iplrrqvsad ssasmnsgvl lvrpsrlsss gtpmlagvse yelpedprwe lprdrlvlgk |
| 481 | plgegcfgqv vlaeaigldk dkpnrvtkva vkmlksdate kdlsdlisem emmkmigkhk |
| 541 | niinllgact qdgplyvive yaskgnlrey lqarrppgle ycynpshnpe eqlsskdlvs |
| 601 | cayqvargme ylaskkcihr dlaarnvlvt ednvmkiadf glardihhid yykkttngrl |
| 661 | pvkwmapeal fdriythqsd vwsfgvllwe iftlggspyp gvpveelfkl lkeghrmdkp |
| 721 | snctnelymm mrdcwhavps qrptfkqlve dldrivalts nqeyldlsmp ldqyspsfpd |
| 781 | trsstcssge dsvfsheplp eepclprhpa qlangglkrr |

By “FGFR1 polynucleotide” is meant a polynucleotide encoding a FGFR1 polypeptide. An exemplary FGFR1 polynucleotide sequence is provided at GenBank Accession No. BC015035.1. The exemplary sequence provided at GenBank Accession No. BC015035.1 is reproduced below (SEQ ID No: 22).

| | |
|---|---|
| 1 | agcgctcttg cggccacagg cgcggcgtcc tcggcggcgg gcggcagcta gcgggagccg |
| 61 | ggacgccggt gcagccgcag cgcgcggagg aacccgggtg tgccgggagc tgggcggcca |
| 121 | cgtccggacg ggaccgagac ccctcgtagc gcattgcggc gacctcgcct tccccggccg |
| 181 | cgagcgcgcc gctgcttgaa aagccgcgga acccaaggac ttttctccgg tccgagctcg |
| 241 | gggcgccccg cagggcgcac ggtacccgtg ctgcagtcgg gcacgccgcg gcgccggggc |
| 301 | ctccgcaggg cgatggagcc cggtctgcaa ggaaagtgag gcgccgccgc tgcgttctgg |
| 361 | aggagggggg caccagctcc ggctccattg ttcccgcccg ggctggaggc gccgagcacc |
| 421 | gagcgccgcc gggagtcgag cgccggccgc ggagctcttg cgaccccgcc aggacccgaa |
| 481 | cagagcccgg gggcggcggg ccggagccgg ggacgcgggc acacgcccgc tcgcacaagc |
| 541 | cacggcggac tctcccgagg cggaacctcc acgccgagcg agggtcagtt tgaaaaggag |
| 601 | gatcgagctc actgtggagt atccatggag atgtggagcc ttgtcaccaa cctctaactg |
| 661 | cagaactggg atgtggagct ggaagtgcct cctcttctgg gctgtgctgg tcacagccac |
| 721 | actctgcacc gctaggccgt ccccgacctt gcctgaacaa gcccagccct ggggagcccc |
| 781 | tgtggaagtg gagtccttcc tggtccaccc cggtgacctg ctgcagcttc gctgtcggct |
| 841 | gcgggacgat gtgcagagca tcaactggct gcgggacggg gtgcagctgg cggaaagcaa |
| 901 | ccgcacccgc atcacagggg aggaggtgga ggtgcaggac tccgtgcccg cagactccgg |
| 961 | cctctatgct tgcgtaacca gcagcccctc gggcagtgac accacctact tctccgtcaa |
| 1021 | tgtttcagat gctctcccct cctcggagga tgatgatgat gatgatgact cctcttcaga |
| 1081 | ggagaaagaa acagataaca ccaaaccaaa ccgtatgccc gtagctccat attggacatc |
| 1141 | cccagaaaag atggaaaaga aattgcatgc agtgccggct gccaagacag tgaagttcaa |
| 1201 | atgcccttcc agtgggaccc caaaccccac actgcgctgg ttgaaaaatg gcaaagaatt |
| 1261 | caaacctgac cacagaattg gaggctacaa ggtccgttat gccacctgga gcatcataat |
| 1321 | ggactctgtg gtgccctctg acaagggcaa ctacacctgc attgtggaga atgagtacgg |
| 1381 | cagcatcaac cacacatacc agctggatgt cgtggagcgg tcccctcacc ggcccatcct |
| 1441 | gcaagcaggg ttgcccgcca acaaaacagt ggccctgggt agcaacgtgg agttcatgtg |
| 1501 | taaggtgtac agtgacccgc agccgcacat ccagtggcta aagcacatcg aggtgaatgg |
| 1561 | gagcaagatt ggcccagaca acctgcctta tgtccagatc ttgaagactg ctggagttaa |
| 1621 | taccaccgac aaagagatgg aggtgcttca cttaagaaat gtctcctttg aggacgcagg |
| 1681 | ggagtatacg tgcttggcgg gtaactctat cggactctcc catcactctg catggttgac |
| 1741 | cgttctggaa gccctggaag agaggccggc agtgatgacc tcgcccctgt acctggagat |
| 1801 | catcatctat tgcacagggg ccttcctcat ctcctgcatg gtggggtcgg tcatcgtcta |
| 1861 | caagatgaag agtggtacca agaagagtga cttccacagc cagatggctg tgcacaagct |
| 1921 | ggccaagagc atccctctgc gcagacaggt gtctgctgac tccagtgcat ccatgaactc |
| 1981 | tggggttctt ctggttcggc catcacggct ctcctccagt gggactccca tgctagcagg |
| 2041 | ggtctctgag tatgagcttc ccgaagaccc tcgctgggag ctgcctcggg acagactggt |
| 2101 | cttaggcaaa cccctgggag agggctgctt tgggcaggtg gtgttggcag aggctatcgg |
| 2161 | gctggacaag gacaaaccca accgtgtgac caaagtggct gtgaagatgt tgaagtcgga |
| 2221 | cgcaacagag aaagacttgt cagacctgat ctcagaaatg gagatgatga agatgatcgg |
| 2281 | gaagcataag aatatcatca acctgctggg ggcctgcacg caggatggtc ccttgtatgt |
| 2341 | catcgtggag tatgcctcca agggcaacct gcgggagtac ctgcaggccc ggaggccccc |
| 2401 | agggctggaa tactgctaca accccagcca caacccagag gagcagctct cctccaagga |
| 2461 | cctggtgtcc tgcgcctacc aggtggcccg aggcatggag tatctggcct ccaagaagtg |
| 2521 | catacaccga gacctggcag ccaggaatgt cctggtgaca gaggacaatg tgatgaagat |
| 2581 | agcagacttt ggcctcgcac gggacattca ccacatcgac tactataaaa agacaaccaa |
| 2641 | cggccgactg cctgtgaagt ggatggcacc cgaggcatta tttgaccgga tctacaccca |
| 2701 | ccagagtgat gtgtggtctt tcggggtgct cctgtgggag atcttcactc tgggcggctc |
| 2761 | cccatacccc ggtgtgcctg tggaggaact tttcaagctg ctgaaggagg gtcaccgcat |
| 2821 | ggacaagccc agtaactgca ccaacgagct gtacatgatg atgcgggact gctggcatgc |
| 2881 | agtgccctca cagagaccca ccttcaagca gctggtggaa gacctggacc gcatcgtggc |
| 2941 | cttgacctcc aaccaggagt acctggacct gtccatgccc ctggaccagt actcccccag |
| 3001 | ctttcccgac acccggagct ctacgtgctc ctcaggggag gattccgtct tctctcatga |
| 3061 | gccgctgccc gaggagccct gcctgccccg acacccagcc cagcttgcca atggcggact |
| 3121 | caaacgccgc tgactgccac ccacacgccc tccccagact ccaccgtcag ctgtaaccct |
| 3181 | cacccacagc ccctgctggg cccaccacct gtccgtccct gtcccctttc ctgctggcag |
| 3241 | gagccggctg cctaccaggg gccttcctgt gtggcctgcc ttcaccccac tcagctcacc |
| 3301 | tctccctcca cctcctctcc acctgctggt gagaggtgca aagaggcaga tctttgctgc |
| 3361 | cagccacttc atcccctccc agatgttgga ccaacacccc tccctgccac caggcactgc |
| 3421 | ctggagggca gggagtggga gccaatgaac aggcatgcaa gtgagagctt cctgagcttt |
| 3481 | ctcctgtcgg tttggtctgt tttgccttca cccataagcc cctcgcactc tggtggcagg |
| 3541 | tgccttgtcc tcagggctac agcagtaggg aggtcagtgc ttcgtgcctc gattgaaggt |
| 3601 | gacctctgcc ccagataggt ggtgccagtg gcttattaat tccgatacta gtttgctttg |
| 3661 | ctgaccaaat gcctggtacc agaggatggt gaggcgaagg ccaggttggg ggcagtgttg |
| 3721 | tggccctggg gcccagcccc aaactggggg ctctgtatat agctatgaag aaaacacaaa |
| 3781 | gtgtataaat ctgagtatat atttacatgt ctttttaaaa gggtcgttac cagagattta |
| 3841 | cccatcgggt aagatgctcc tggtggctgg gaggcatcag ttgctatata ttaaaaacaa |
| 3901 | aaaaaaaaaa aaa |

By “FRS2α polypeptide” is meant a polypeptide or fragment thereof having at least about 85% amino acid identity to NCBI Accession No. NP_001265286.1 and having a biological activity of a FRS2α polypeptide. Biological activities of a FRS2α polypeptide include transmembrane receptor protein tyrosine kinase adaptor activity and binding to a FGFR1 polypeptide. The sequence at NCBI Accession No. NP_001265286.1 is shown below (SEQ ID No: 23):

| | |
|---|---|
| 1 | mgsccscpdk dtvpdnhrnk fkvinvdddg nelgsgimel |
| | tdtelilytr krdsvkwhyl |
| 61 | clrrygydsn lfsfesgrrc qtgqgifafk caraeelfnm |
| | lqeimqnnsi nvveepvver |
| 121 | nnhqtelevp rtprtpttpg faaqnlpngy prypsfgdas |
| | shpssrhpsv gsarlpsvge |
| 181 | esthpllvae eqvhtyvntt gvqeerknrt svhvplearv |
| | snaesstpke epssiedrdp |
| 241 | qillepegvk fvlgptpvqk qlmekekleq igrdqvsgsg |
| | anntewdtgy dsderrdaps |
| 301 | vnklvyenin glsipsasgv rrgrltstst sdtqninnsa |
| | qrrtallnye nlpslppvwe |
| 361 | arklsrdedd nlgpktpsln gyhnnldpmh nyvntenvtv |
| | pasahkieys rrrdctptvf |
| 421 | nfdirrpsle hrqlnyiqvd leggsdsdnp qtpktpttpl |
| | pqtptrrtel yavidierta |
| 481 | amsnlqkalp rddgtsrktr hnstdlpm |

By “FRS2α polynucleotide” is meant a polynucleotide encoding a FRS2α polypeptide. An exemplary FRS2α polynucleotide sequence is provided at NCBI Accession No. NM_001278357.1. The exemplary sequence provided at NCBI Accession No. NM_001278357.1 is reproduced below (SEQ ID No: 24).

| | |
|---|---|
| 1 | aaaacccttc cctcccccgc tcccccggaa gtgcttttcc aagattcggg ccggagagag |
| 61 | gccttgtagg cacagcggct gagactcgat ctgctccaag taggggctcc agcgcgggtc |
| 121 | ggagtctggg ggttcgcgcc cgccgacccg cgccctgctc cctctcagca cctgggcgga |
| 181 | cggttaaatc agcaaacaaa gaaaacatgg tattttgaaa tatgattaaa ctcctgatgc |
| 241 | tgcagcagag gctaagaata ttaatggcca gatctagtgc acacatggtc ttctgaagaa |
| 301 | gccatgggta gctgttgtag ctgtccagat aaagacactg tcccagataa ccatcggaac |
| 361 | aagtttaagg tcattaatgt ggatgatgat gggaatgagt taggttctgg cataatggaa |
| 421 | cttacagaca cagaactgat tttatacacc cgcaaacgtg actcagtaaa atggcactac |
| 481 | ctctgcctgc gacgctatgg ctatgactcg aatctctttt cttttgaaag tggtcgaagg |
| 541 | tgtcaaactg gacaaggaat ctttgccttt aagtgtgccc gtgcagaaga attatttaac |
| 601 | atgttgcaag agattatgca aaataatagt ataaatgtgg tggaagagcc agttgtagaa |
| 661 | agaaataatc atcagacaga attggaagtc cctagaacac ctcgaacacc tacaactcca |
| 721 | ggatttgctg ctcagaactt acctaatgga tatccccgat atccctcatt tggagatgct |
| 781 | tcatcccatc cgtcaagcag acatccttct gtgggaagtg ctcgcctgcc ttcagtaggg |
| 841 | gaagaatcta cacatccttt gcttgtggct gaggaacaag tacataccta tgtcaacact |
| 901 | acaggtgtgc aagaagagcg gaaaaaccgc acaagtgtgc atgttccatt ggaggcgagg |
| 961 | gtttctaacg ctgaaagcag cacaccaaaa gaagaaccaa gtagtattga ggacagggat |
| 1021 | cctcagattc ttcttgaacc tgaaggagtc aaatttgttt tagggccaac ccctgttcaa |
| 1081 | aagcagttaa tggaaaaaga gaaactggag caacttggaa gagatcaagt tagtggaagt |
| 1141 | ggagcaaata acacagaatg ggacactggc tatgacagtg atgaacgaag agatgcaccc |
| 1201 | tctgttaaca aactggtgta tgaaaatata aatgggctat ctatccctag tgcctcaggg |
| 1261 | gtcaggagag gtcgtctgac atccaccagt acctcagata cccagaatat caacaactca |
| 1321 | gctcagagaa gaactgcatt attaaactat gaaaatctac catctttgcc tcctgtttgg |
| 1381 | gaagcccgca agctaagtag ggatgaagat gacaatttag gaccaaagac cccatctcta |
| 1441 | aatggctacc ataataatct agatccaatg cataactatg taaatacaga gaatgtaaca |
| 1501 | gtgccagcaa gtgctcacaa aatagaatat tcaaggcgtc gggactgtac accaacagtc |
| 1561 | tttaactttg atatcagacg cccaagttta gaacacaggc agcttaatta catacaggtt |
| 1621 | gacttggaag gtggcagtga ctctgacaac cctcagactc caaaaacgcc tacaactccc |
| 1681 | cttccacaaa cccctaccag gcgcacagag ctgtatgccg tgatagacat cgagagaact |
| 1741 | gctgctatgt caaatttgca gaaagcactg ccacgagatg atggtacatc taggaaaact |
| 1801 | agacacaata gtactgatct gcccatgtga gcctggaaag cattgtgttg tttgcacctt |
| 1861 | tgtgaagttt ttaaaaatga agatgcaagt gcttcatttt catttctaaa cactaactcc |
| 1921 | ttttatagac tgataaaatt tttttctgaa tatttcatgt gcatctttaa ctaaagggaa |
| 1981 | ttaatgtaga gcaggtactc cttaaagaac actaatttca ttatatacta ctcgttgtac |
| 2041 | agcagcattc ccgttttcac agtgcctatt taaaatgaga gttgaagtaa atgacatgct |
| 2101 | ggttgatttt tatcaatatt ctggacttaa cgcatacctt tcatgtctaa gtcatggttg |
| 2161 | gcttttaaaa ctttttataa agcctcttga caatgtacat tgctaacagg taactatagg |
| 2221 | ctttgaaagt aatgctcgta gattcagtgt tcacagtatg tggcctccag catgtaacat |
| 2281 | gaggaatcct ttatttcatt aattaatggc tttttgactt gagccaaaac atatgtaaag |
| 2341 | gaaacagaag taccgcacct cctcttacac cagtcagctc ctttgccttc agtgttacta |
| 2401 | gaaagcggcc tgtgtccatg agtgtgcttt gctgttggtg cactgaaagg caggaaggag |
| 2461 | acaagatttt ctatttactc atctcatgat gtcatttgaa gggcatgtcc agatatctta |
| 2521 | aaattataat aggctcaaga atcagtctca ggtcacttta cccaaaaaca tttgaaaatc |
| 2581 | tgaaccacaa tctcctgaaa gtttttctcc tatagattgt tgacaacaca ttgttttctg |
| 2641 | gaggcatttg tgccattagg tttccattta tcttcagttt ttttctttgg tgtttgggat |
| 2701 | gtcttatttt gttgccttat gtccttttca atttaaaatg tttgagtttg tatatagttt |
| 2761 | tgaaattgga ttatgtgttc attgttgttt agtttgcatt tttgtcaaat tatggttttg |
| 2821 | aaggttcatt tggaacttac tgttagtctg taacagggtt gcccttgtcc agtatttatt |
| 2881 | tataagctgt ttacttttca agttgataaa aacattctcc aattctaaat ttgcttgtgt |
| 2941 | ccataggtga tctctttagc aaactgagaa aaaaaggaag ctacttttaa catgcaaagt |
| 3001 | tccctcaagg tgtaccgtgt tgtctctgtg ggcactcttc cccagcactt tagcagtaat |
| 3061 | tcccccagct acacgctgca gttgtactct gcccactcta gtgttcctca gctctgctgt |
| 3121 | ccttttactt gtagctggat ctttgattat ccttcgattt ccatgaaata ttaatattgt |
| 3181 | tgccagcata gcaggtacag tggaagtctt gtagcagtga gattgtatca taatttagga |
| 3241 | tttaaaatga attaaagttt atataaactg aagagtctcc atatgtcaaa ctcttggaaa |
| 3301 | atcaaagatg ttccaatttc ctaaacacta gagaatacga gagaaggtag agtggaaaag |
| 3361 | gttaggtaac cttgcaaaat attttactat tttctctaaa tatgaggaag tttgagatta |
| 3421 | tgatctggat ctaccagata taactaaggt taatttagca tgaaaaagtt ttagtcatat |
| 3481 | tggcatccaa cctattcagt aaccgaatca taggacaatg atggattagg agaacaatag |
| 3541 | agtgggatca ttataaagaa aataaattat taaaggtgtc tttatcgttt tagtgccatt |
| 3601 | tttagtgtct ttactataaa tcaatatcag tgtattttat cattctatgt gcatagcaga |
| 3661 | attttctttt ctcccttttg ttcccctgtg aacttggtgc ttattaaagt gctcactgtt |
| 3721 | ctcttaaaag agagcagtgg tataggtgtg cagtttccat gatgcaggtt ccatttttaa |
| 3781 | tatattgttc cacttatcct ttcttctgag taaattgcta attgtgccaa atttatgtaa |
| 3841 | tagtttttgt aatgtggaat aagaattatg atggaaccat tgcacatttt tttctgaaac |
| 3901 | agccagtcaa ggcagaacat taatctccaa atgcaagggc tgatctattt attcattttg |
| 3961 | gaggttgggt actttattct ttctttccgt catccttttc attgtttccc ccggattcta |
| 4021 | attagttttt atttttttta gataactcca atataatcat tacagtttat gctttaaata |
| 4081 | ctatgtgctt taaaaaggaa aatgggacca atttgtctgc taagaatttg attttaggta |
| 4141 | ctataagagt attaggaaaa tatatacaac tggtgttaat ttctagatat tttctagaaa |
| 4201 | tcacttgtgt tcctatttaa taaaaggtaa tttagaatac tacttgtcct ttgcagtagt |
| 4261 | ttagtaatgg gcattaagct gtgtcctcga aggatgtacc tattactagg tgcattttag |
| 4321 | aatgaaatat tgatatttta ttagcatata attgtggcca tatatctcag attttctgag |
| 4381 | gcagatctaa ttttagataa ttctgttggt agaccatgtg atccttcttt ttggttttgg |
| 4441 | aaatataatc attgttaatg ttttccctcc aaatagaata ctgttttatc catacaaatc |
| 4501 | ataacagcat ctatcccatg ctagggttgg aaactgatat tggtattact tgtgtttttt |
| 4561 | cttagtgtgt tttatttccc agtttcatct tcttctaaaa atgaaaatat ggtgccttcc |
| 4621 | ctccctccag gaagactggc aaatatttcc ttttatttac tgctgctgtg gagtgatgag |
| 4681 | atatgcactt tactctttaa gattcagcaa aaagcttttc acttctcagt atatccagaa |
| 4741 | tacatcatat ctgggactta ggaaaatttg ccaagcaatc tttgttttta tagatactaa |
| 4801 | tgttgaccct ctccagcgtt caatgttata aatagaacaa gtcaagctag tgtttatctc |
| 4861 | ctccccctcc ccaaaactgt ggcacagcat ataaaaatgt acctcaataa tgttctatta |
| 4921 | aaaatgggac aggggcctta tgttttcata atttcccaac aatgtgccgc catatttttg |
| 4981 | cctcaaggta aaggttttaa cagatgaaaa agtacttccc aattcccccg tgctattcct |
| 5041 | aacctataat gcccaaatgt tttgtgcaat gtgtagtgtg tgtgtataaa tacatatatt |
| 5101 | cttgaaatag acataccatc agagacatca ttcacaagta actgatgtat tggcatctca |
| 5161 | ttcatatttc tgatgtgtga ggtatatggt actaattacc ttttccttga tgtttgccaa |
| 5221 | atttgaataa aggcattggt acgaaattac agaatgtaaa gaaaatgttt ttggcttgaa |
| 5281 | aaattaacat attttatgac gtaccacagt atactctgcc caaaccagca ccctatctat |
| 5341 | ctttcctgtt ctttacatcc ctgttcccca tccctacttc ctcatttttg gtataacaca |
| 5401 | gttcttttgt agcatcatta taattgcagt tctatggcaa ttggacagtt atagcatgga |
| 5461 | aacagactgg tataagtagt acagtagtca ccagtgtgcc acatttgcat tagtaatgca |
| 5521 | aaatatacat tttataaagg acaaactttg tgttatgttt tattttcatt acattgtata |
| 5581 | atattgtaag actattgtat gtcctaattt gcattataaa tgtttttttc ctacgtaaag |
| 5641 | gcataaatat agcaactttg tataaaggta gcttattaga tttttaattt tttcttttat |
| 5701 | aaaaaattgt ccaacagtgg gactaccatt gccaaattgt atatgaaata tgaattttac |
| 5761 | ccccatggtt aatttctttt ataaacattc catatttctc taataaaaag acataagtga |
| 5821 | tactgtacta tgcatacatt gtatcttaat gctgtttcag atcagcattt taaattttgg |
| 5881 | tttgcatttt taatattggc aaaacgtaac cactgttaat taaaataaaa ccttgttgta |
| 5941 | tatgtaacaa cataattttc cctctatccc ttcccaccct ttgttctcta tttctcccta |
| 6001 | tcagtgccaa cttcatacat tttgtagcat ggcaataaaa tataactttt acactgaggc |
| 6061 | cgagtgtggc tttttggagg aagtggggat gggacgattg ccctctagtt gtcctttgca |
| 6121 | tatgactgtt ttttgccata taagccatgt catcaggcat gaaaagtttt ctcatatatg |
| 6181 | atgtaaactt gcttttaagg acaagtgtga atgtgctttt taagcttaat ttttgtcatg |
| 6241 | acaactaatt ttttttatct ttggagaagt cagagttctt tacaatcaaa cgtttattaa |
| 6301 | ctggagtact tagaataagc tagtaattga atttagttca agggctaagc aacacatttt |
| 6361 | taaatcctta tttattgtag agtattagta tactgtccta caaattatgt aaaatatggt |
| 6421 | ttaatattag atgactttgg attttgcaat gccttactgt tgtcattcta gcataaatat |
| 6481 | ccataatgag gtactcaagt tgatactgga agctgagctg atcatacact gacctgaagc |
| 6541 | attcatgaaa agctgcttta ttgaataaag tctgattgga gttcttttca tgctcacttt |
| 6601 | ccccttattg ctgaaagtag attgcaataa aaccccaata aaacgtttgg tcggatatct |
| 6661 | acttaaaaaa aaaaaa |

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “expression” as used herein is defined as the transcription and/or translation of a particular nucleotide sequence driven by its promoter.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation can be at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any polypeptide or polynucleotide having an alteration in expression level, sequence, or activity that is associated with a disease or disorder or risk of disease or disorder. In some embodiments, a decrease in activity or level of a FGF signaling polypeptide or let-7 miRNA in an endothelial cell is associated with development and/or progression of atherosclerosis. In some embodiments, an increase in level or activity of a TGFβ signaling polypeptide (e.g., TGFβ 1, TGFβ2, TGFβ3, TGFβR1, TGFβR2) in an endothelial cell is associated with development and/or progression of atherosclerosis. In some other embodiments, an increase in activity or level of a FGF signaling polypeptide or let-7 miRNA in a smooth muscle cell is associated with development and/or progression of atherosclerosis. In still other embodiments, a decrease in level or activity of a TGFβ signaling polypeptide (e.g., TGFβ 1, TGFβ2, TGFβ3, TGFβR1, TGFβR2) is associated with development and/or progression of atherosclerosis.

As used herein, “microRNA” or “miRNA” describes small non-coding RNA molecules, generally about 15 to about 50 nucleotides in length, preferably 17-23 nucleotides, 15 which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from 20 primary transcripts (pri-miRNA) through sequential cleavage by RNAse III enzymes. miRBase is a comprehensive microRNA database located at www.mirbase.org, incorporated by reference herein in its entirety for all purposes.

By “let-7 miRNA” is meant a miRNA member of the let-7 miRNA family. Sequences of members of the let-7 miRNA family can be found in, for example, www.mirbase.org. Exemplary members of the let-7 miRNA family include hsa-let-7b or human let-7b (miRBase Accession No. MI0000063), hsa-let-7a-1 (miRBase Accession No. MI0000060), hsa-let-7a-2 (miRBase Accession No. MI0000061), hsa-let-7a-3 (miRBase Accession No. MI0000062), hsa-let-7b, hsa-let-7c (miRBase Accession No. MI0000064), hsa-let-7d (miRBase Accession No. MI0000065), hsa-let-7e (miRBase Accession No. MI0000066), hsa-let-7f-1 (miRBase Accession No. MI0000067), hsa-let-7f-2 (miRBase Accession No. MI0000068), hsa-let-7g (miRBase Accession No. M10000433), and hsa-let-7i (miRBase Accession No. MI00000434). The sequence of human let-7b provided at miRBase Accession No. MI0000063 is reproduced below.

human let-7b (5 prime):
(SEQ ID No: 19)
UGAGGUAGUAGGUUGUGUGGUU
human let-7b (3 prime):
(SEQ ID No: 20)
CUAUACAACCUACUGCCUUCCC

The let-7 miRNA family has been shown to play important roles in animal development, cell differentiation, and metabolism. In some embodiments, an activity of let-7 miRNA is repression of expression of a TGFβ signaling polypeptide. In some embodiments, an activity of let-7 miRNA is repression of TGFβ signaling.

In some embodiments, the let-7 miRNA is used as a therapeutic. Use of let-7 miRNA as a therapeutic has been demonstrated previously. For example, let-7 miRNA was used as anti-cancer therapy (Trang et al., Mol Ther. 2011 June; 19(6): 1116-1122).

In some embodiments, the let-7 miRNA is chemically modified. In particular embodiments, uracil (“U”) or cytosine (“C”) is chemically modified. In some embodiments, the miRNA is modified to impart properties to the miRNA to make it useful as a therapeutic, such as attenuated immunostimulation and increased serum stability. Such modifications to the miRNA include, without limitation, incorporation of a 2′-O-methyl (2′-O-Me), phosphorothioate (PS), and deoxy thymidine (dT) residues. In particular embodiments, the modified miRNA retains silencing activity in vivo. In particular embodiments, the modification is a 2′-O-methyl nucleotide modification. In some embodiments, the modification decreases the likelihood of triggering an innate immune response.

In some embodiments, the let-7 miRNA contains a “light” modification. By a miRNA containing a “light modification” is meant that the miRNA contains a 2′-O-methyl modification on all U and C nucleotide bases followed by adenosine (“A”) on the antisense strand. In some other embodiments, the let-7 miRNA contains a “heavy” modification. By a miRNA containing a “heavy modification” is meant that the miRNA contains a 2′-O-methyl modification on all U and C nucleotide bases on the sense strand.

In still other embodiments, the let-7 miRNA is “mi-let-7b_{L}”. mi-let-7b_{L} is also referred to herein as “let-7 light.” The sequence of mi-let-7b_{L} is provided below:

mi-let-7b_{L} (5 prime):
(SEQ ID No: 19)
UGAGGuAGuAGGUUGUGUGGUU
mi-let-7b_{L} (3 prime):
(SEQ ID NO: 20)
CuAuAcAACCuACUGCCUUCCC

In some other embodiments, the let-7 miRNA is “mi-let-7b_{H}”. mi-let-7b_{H} is also referred to herein as “let-7 heavy.” The sequence of mi-let-7b_{H} miRNA is provided below: mi-let-7b_{H} (5 prime): UGAGGuAGuAGGUUGUGUGGUU (SEQ ID No: 19) mi-let-7b_{H} (3 prime): cuAuAcAAccuAcuGccuuccc (SEQ ID NO: 20) In the foregoing sequences, lower case indicates a nucleotide base containing a 2′-O-methyl modification.

As used herein, “obtaining” as in “obtaining an agent” includes synthesizing, purchasing, or otherwise acquiring the agent.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, “polynucleotide” includes cDNA, RNA, DNA/RNA hybrid, antisense RNA, siRNA, miRNA, snoRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the term “promoter” or “regulatory sequence” means a nucleic acid sequence which is required for expression of a gene product operably linked to the promoter or regulator sequence. In some instances, this sequence may be the core promoter sequence and in other instances, this sequence may also include an enhancer sequence and other regulatory elements which are required for expression of the gene product. The promoter or regulatory sequence may, for example, be one which expresses the gene product in an inducible manner.

By “reduces” is meant a negative alteration of at least 10%, 25%, 50%, 75%, or 100%.

By “reference” is meant a standard or control condition. In some embodiments, the reference is an activity or level of a TGFβ signaling polypeptide or polynucleotide or a FGF signaling polypeptide or polynucleotide in a healthy, normal subject or in a subject that does not have atherosclerosis. In some embodiments, the reference is an activity or level of a let-7 miRNA in a healthy, normal subject or in a subject that does not have atherosclerosis. In some embodiments, the TGFβ signaling polypeptide or polynucleotide is a TGFβ 1, TGFβ2, TGFβ3, TGFβR1, or TGFβR2 polypeptide or polynucleotide. In some embodiments, the FGF signaling polypeptide is FRS2α. In some other embodiments, the let-7 miRNA is at least one selected from the group consisting of human let-7b miRNA and human let-7c miRNA.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, or at least about 25 amino acids. The length of the reference polypeptide sequence can be about 35 amino acids, about 50 amino acids, or about 100 amino acids. For nucleic acids, the length of the reference nucleic acid sequence will generally be at least about 50 nucleotides, at least about 60 nucleotides, or at least about 75 nucleotides. The length of the reference nucleic acid sequence can be about 100 nucleotides, about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant an agent that recognizes and binds a polypeptide or polynucleotide of the invention, but which does not substantially recognize and bind other molecules in a sample, for example, a biological sample, which naturally includes a polynucleotide of the invention. In some embodiments, the agent is a nucleic acid molecule.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., and at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 g/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., and at least about 68° C. In one embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, at least 80%, at least 85%, at least 90%, at least 95% or even at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, a “TGFβ signaling polypeptide” refers to a member or component of a transformation growth factor β (TGFβ) signaling pathway. Exemplary TGFβ signaling polypeptides include polypeptides TGFβ1, TGFβ2, TGFβ3, TGFβR1, TGFβR2, SMAD1, SMAD2, SMAD3, SMAD4, SMAD5, and SMAD9.

As used herein, a “TGFβ signaling polynucleotide” is a polynucleotide encoding a TGFβ signaling polypeptide.

By “TGFβ1 polypeptide” is meant a polypeptide or fragment thereof having at least about 85% amino acid identity to GenBank Accession No. AAH22242.1 and having a biological activity of a TGFβ1 polypeptide. Biological activities of a TGFβ1 polypeptide include binding to a type II transforming growth factor β (TGFβ) receptor and homodimerization. The sequence at GenBank Accession No. AAH22242.1 is shown below (SEQ ID NO: 25):

| | |
|---|---|
| 1 | mppsglrlll lllpllwllv ltpgrpaagl stcktidmel |
| | vkrkrieair gqilsklrla |
| 61 | sppsqgevpp gplpeavlal ynstrdrvag esaepepepe |
| | adyyakevtr vlmvethnei |
| 121 | ydkfkqsths iymffntsel reavpepvll sraelrllrl |
| | klkveqhvel yqkysnnswr |
| 181 | ylsnrllaps dspewlsfdv tgvvrqwlsr ggeiegfrls |
| | ahcscdsrdn tlqvdingft |
| 241 | tgrrgdlati hgmnrpfill matpleraqh lqssrhrral |
| | dtnycfsste knccvrqlyi |
| 301 | dfrkdlgwkw ihepkgyhan fclgpcpyiw sldtqyskvl |
| | alynqhnpga saapccvpqa |
| 361 | leplpivyyv grkpkveqls nmivrsckcs |

By “TGFβ1 polynucleotide” is meant a polynucleotide encoding a TGFβ1 polypeptide. An exemplary TGFβ1 polynucleotide sequence is provided at GenBank Accession No. BC022242.1. The exemplary sequence provided at GenBank Accession No. BC022242.1 is reproduced below (SEQ ID NO: 26).

| | |
|---|---|
| 1 | cccagacctc gggcgcaccc cctgcacgcc gccttcatcc ccggcctgtc tcctgagccc |
| 61 | ccgcgcatcc tagacccttt ctcctccagg agacggatct ctctccgacc tgccacagat |
| 121 | cccctattca agaccaccca ccttctggta ccagatcgcg cccatctagg ttatttccgt |
| 181 | gggatactga gacacccccg gtccaagcct cccctccacc actgcgccct tctccctgag |
| 241 | gacctcagct ttccctcgag gccctcctac cttttgccgg gagaccccca gcccctgcag |
| 301 | gggcggggcc tccccaccac accagccctg ttcgcgctct cggcagtgcc ggggggcgcc |
| 361 | gcctccccca tgccgccctc cgggctgcgg ctgctgctgc tgctgctacc gctgctgtgg |
| 421 | ctactggtgc tgacgcctgg ccggccggcc gcgggactat ccacctgcaa gactatcgac |
| 481 | atggagctgg tgaagcggaa gcgcatcgag gccatccgcg gccagatcct gtccaagctg |
| 541 | cggctcgcca gccccccgag ccagggggag gtgccgcccg gcccgctgcc cgaggccgtg |
| 601 | ctcgccctgt acaacagcac ccgcgaccgg gtggccgggg agagtgcaga accggagccc |
| 661 | gagcctgagg ccgactacta cgccaaggag gtcacccgcg tgctaatggt ggaaacccac |
| 721 | aacgaaatct atgacaagtt caagcagagt acacacagca tatatatgtt cttcaacaca |
| 781 | tcagagctcc gagaagcggt acctgaaccc gtgttgctct cccgggcaga gctgcgtctg |
| 841 | ctgaggctca agttaaaagt ggagcagcac gtggagctgt accagaaata cagcaacaat |
| 901 | tcctggcgat acctcagcaa ccggctgctg gcacccagcg actcgccaga gtggttatct |
| 961 | tttgatgtca ccggagttgt gcggcagtgg ttgagccgtg gaggggaaat tgagggcttt |
| 1021 | cgccttagcg cccactgctc ctgtgacagc agggataaca cactgcaagt ggacatcaac |
| 1081 | gggttcacta ccggccgccg aggtgacctg gccaccattc atggcatgaa ccggcctttc |
| 1141 | ctgcttctca tggccacccc gctggagagg gcccagcatc tgcaaagctc ccggcaccgc |
| 1201 | cgagccctgg acaccaacta ttgcttcagc tccacggaga agaactgctg cgtgcggcag |
| 1261 | ctgtacattg acttccgcaa ggacctcggc tggaagtgga tccacgagcc caagggctac |
| 1321 | catgccaact tctgcctcgg gccctgcccc tacatttgga gcctggacac gcagtacagc |
| 1381 | aaggtcctgg ccctgtacaa ccagcataac ccgggcgcct cggcggcgcc gtgctgcgtg |
| 1441 | ccgcaggcgc tggagccgct gcccatcgtg tactacgtgg gccgcaagcc caaggtggag |
| 1501 | cagctgtcca acatgatcgt gcgctcctgc aagtgcagct gaggtcccgc cccgccccgc |
| 1561 | cccgccccgg caggcccggc cccaccccgc cccgcccccg ctgccttgcc catgggggct |
| 1621 | gtatttaagg acacccgtgc cccaagccca cctggggccc cattaaagat ggagagagga |
| 1681 | aaaaaaaaaa aaaaaaaaaa aaaaaaaaaa aaaaaaaaaa aaaaaaaaaa aaaaaaaaaa |
| 1741 | aaaaaa |

By “TGFβ2 polypeptide” is meant a polypeptide or fragment thereof having at least about 85% amino acid identity to GenBank Accession No. AAA50405.1 and having a biological activity of a TGFβ2 polypeptide. Biological activities of a TGFβ2 polypeptide include binding to a type II transforming growth factor β (TGFβ) receptor and homodimerization. The sequence at GenBank Accession No. AAA50405.1 is shown below (SEQ ID NO: 27):

| | |
|---|---|
| 1 | mhycvlsafl ilhlvtvals istcstldmd qfmrkrieai |
| | rgqilsklkl tsppedypep |
| 61 | eevppevisi ynstrdllqe kasrraaace rersdeeyya |
| | kevykidmpp ffpseaippt |
| 121 | fyrpyfrivr fdvsamekna snlvkaefrv frlqnpkarv |
| | peqrielyqi lkskdltspt |
| 181 | qryidskvvk traegewlsf dvtdavhewl hhkdrnlgfk |
| | islhcpcctf vpsnnyiipn |
| 241 | kseelearfa gidgtstyts gdqktikstr kknsgktphl |
| | llmllpsyrl esqqtnrrkk |
| 301 | raldaaycfr nvqdncclrp lyidfkrdlg wkwihepkgy |
| | nanfcagacp ylwssdtqhs |
| 361 | rvlslyntin peasaspccv sqdlepltil yyigktpkie |
| | qlsnmivksc kcs |

By “TGFβ2 polynucleotide” is meant a polynucleotide encoding a TGFβ2 polypeptide. An exemplary TGFβ2 polynucleotide sequence is provided at GenBank Accession No. M19154.1. The exemplary sequence provided at GenBank Accession No. M19154.1 is reproduced below (SEQ ID NO: 28).

| | |
|---|---|
| 1 | gcccctcccg tcagttcgcc agctgccagc cccgggacct tttcatctct tcccttttgg |
| 61 | ccggaggagc cgagttcaga tccgccactc cgcacccgag actgacacac tgaactccac |
| 121 | ttcctcctct taaatttatt tctacttaat agccactcgt ctcttttttt ccccatctca |
| 181 | ttgctccaag aatttttttc ttcttactcg ccaaagtcag ggttccctct gcccgtcccg |
| 241 | tattaatatt tccacttttg gaactactgg ccttttcttt ttaaaggaat tcaagcagga |
| 301 | tacgtttttc tgttgggcat tgactagatt gtttgcaaaa gtttcgcatc aaaaacaaca |
| 361 | acaacaaaaa accaaacaac tctccttgat ctatactttg agaattgttg atttcttttt |
| 421 | tttattctga cttttaaaaa caactttttt ttccactttt ttaaaaaatg cactactgtg |
| 481 | tgctgagcgc ttttctgatc ctgcatctgg tcacggtcgc gctcagcctg tctacctgca |
| 541 | gcacactcga tatggaccag ttcatgcgca agaggatcga ggcgatccgc gggcagatcc |
| 601 | tgagcaagct gaagctcacc agtcccccag aagactatcc tgagcccgag gaagtccccc |
| 661 | cggaggtgat ttccatctac aacagcacca gggacttgct ccaggagaag gcgagccgga |
| 721 | gggcggccgc ctgcgagcgc gagaggagcg acgaagagta ctacgccaag gaggtttaca |
| 781 | aaatagacat gccgcccttc ttcccctccg aaactgtctg cccagttgtt acaacaccct |
| 841 | ctggctcagt gggcagcttg tgctccagac agtcccaggt gctctgtggg taccttgatg |
| 901 | ccatcccgcc cactttctac agaccctact tcagaattgt tcgatttgac gtctcagcaa |
| 961 | tggagaagaa tgcttccaat ttggtgaaag cagagttcag agtctttcgt ttgcagaacc |
| 1021 | caaaagccag agtgcctgaa caacggattg agctatatca gattctcaag tccaaagatt |
| 1081 | taacatctcc aacccagcgc tacatcgaca gcaaagttgt gaaaacaaga gcagaaggcg |
| 1141 | aatggctctc cttcgatgta actgatgctg ttcatgaatg gcttcaccat aaagacagga |
| 1201 | acctgggatt taaaataagc ttacactgtc cctgctgcac ttttgtacca tctaataatt |
| 1261 | acatcatccc aaataaaagt gaagaactag aagcaagatt tgcaggtatt gatggcacct |
| 1321 | ccacatatac cagtggtgat cagaaaacta taaagtccac taggaaaaaa aacagtggga |
| 1381 | agaccccaca tctcctgcta atgttattgc cctcctacag acttgagtca caacagacca |
| 1441 | accggcggaa gaagcgtgct ttggatgcgg cctattgctt tagaaatgtg caggataatt |
| 1501 | gctgcctacg tccactttac attgatttca agagggatct agggtggaaa tggatacacg |
| 1561 | aacccaaagg gtacaatgcc aacttctgtg ctggagcatg cccgtattta tggagttcag |
| 1621 | acactcagca cagcagggtc ctgagcttat ataataccat aaatccagaa gcatctgctt |
| 1681 | ctccttgctg cgtgtcccaa gatttagaac ctctaaccat tctctactac attggcaaaa |
| 1741 | cacccaagat tgaacagctt tctaatatga ttgtaaagtc ttgcaaatgc agctaaaatt |
| 1801 | cttggaaaag tggcaagacc aaaatgacaa tgatgatgat aatgatgatg acgacgacaa |
| 1861 | cgatgatgct tgtaacaaga aaacataaga gagccttggt tcatcagtgt taaaaaattt |
| 1921 | ttgaaaaggc ggtactagtt cagacacttt ggaagtttgt gttctgtttg ttaaaactgg |
| 1981 | catctgacac aaaaaaagtt gaaggcctta ttctacattt cacctacttt gtaagtgaga |
| 2041 | gagacaagaa gcaaattttt tttaaagaaa aaaataaaca ctggaagaat ttattagtgt |
| 2101 | taattatgtg aacaacgaca acaacaacaa caacaacaaa caggaaaatc ccattaagtg |
| 2161 | gagttgctgt acgtaccgtt cctatcccgc gcctcacttg atttttctgt attgctatgc |
| 2221 | aataggcacc cttcccattc ttactcttag agttaacagt gagttattta ttgtgtgtta |
| 2281 | ctatataatg aacgtttcat tgcccttgga aaataaaaca ggtgtataaa gtggagacca |
| 2341 | aatactttgc cagaaactca tggatggctt aaggaacttg aactcaaacg agccagaaaa |
| 2401 | aaagaggtca tattaatggg atgaaaaccc aagtgagtta ttatatgacc gagaaagtct |
| 2461 | gcattaagat aaagaccctg aaaacacatg ttatgtatca gctgcctaag gaagcttctt |
| 2521 | gtaaggtcca aaaactaaaa agactgttaa taaaagaaac tttcagtcag |

By “TGFβ3 polypeptide” is meant a polypeptide or fragment thereof having at least about 85% amino acid identity to GenBank Accession No. EAW81249.1 and having a biological activity of a TGFβ3 polypeptide. Biological activities of a TGFβ3 polypeptide include binding to a type II transforming growth factor β (TGFβ) receptor and homodimerization. The sequence at GenBank Accession No. EAW81249.1 is shown below (SEQ ID NO: 29):

| | |
|---|---|
| 1 | mkmhlqralv vlallnfatv slslstcttl dfghikkkrv |
| | eairgqilsk irltsppept |
| 61 | vmthvpyqvl alynstrell eemhgereeg ctqentesey |
| | yakeihkfdm iqglaehnel |
| 121 | avcpkgitsk vfrfnvssve knrtnlfrae frvlrvpnps |
| | skrneqriel fqilrpdehi |
| 181 | akqryiggkn iptrgtaewl sfdvtdtvre wllrresnlg |
| | leisihcpch tfqpngdile |
| 241 | nihevmeikf kgvdneddhg rgdlgrlkkq kdhhnphlil |
| | mmipphrldn pgqggqrkkr |
| 301 | aldtnycfrn leenccvrpl yidfrqdlgw kwvhepkgyy |
| | anfcsgpcpy lrsadtthst |
| 361 | vlglyntlnp easaspccvp qdlepltily yvgrtpkveq |
| | isnmvvksck cs |

By “TGFβ3 polynucleotide” is meant a polynucleotide encoding a TGFβ3 polypeptide. An exemplary TGFβ3 polynucleotide sequence is provided at NCBI Accession No. NG_011715.1. The exemplary sequence provided at NCBI Accession No. BT007287.1 is reproduced below (SEQ ID NO: 30).

| | |
|---|---|
| 1 | atgaagatgc acttgcaaag ggctctggtg gtcctggccc |
| | tgctgaactt tgccacggtc |
| 61 | agcctctctc tgtccacttg caccaccttg gacttcggcc |
| | acatcaagaa gaagagggtg |
| 121 | gaagccatta ggggacagat cttgagcaag ctcaggctca |
| | ccagcccccc tgagccaacg |
| 181 | gtgatgaccc acgtccccta tcaggtcctg gccctttaca |
| | acagcacccg ggagctgctg |
| 241 | gaggagatgc atggggagag ggaggaaggc tgcacccagg |
| | aaaacaccga gtcggaatac |
| 301 | tatgccaaag aaatccataa attcgacatg atccaggggc |
| | tggcggagca caacgaactg |
| 361 | gctgtctgcc ctaaaggaat tacctccaag gttttccgct |
| | tcaatgtgtc ctcagtggag |
| 421 | aaaaatagaa ccaacctatt ccgagcagaa ttccgggtct |
| | tgcgggtgcc caaccccagc |
| 481 | tctaagcgga atgagcagag gatcgagctc ttccagatcc |
| | ttcggccaga tgagcacatt |
| 541 | gccaaacagc gctatatcgg tggcaagaat ctgcccacac |
| | ggggcactgc cgagtggctg |
| 601 | tcctttgatg tcactgacac tgtgcgtgag tggctgttga |
| | gaagagagtc caacttaggt |
| 661 | ctagaaatca gcattcactg tccatgtcac acctttcagc |
| | ccaatggaga tatcctggaa |
| 721 | aacattcacg aggtgatgga aatcaaattc aaaggcgtgg |
| | acaatgagga tgaccatggc |
| 781 | cgtggagatc tggggcgcct caagaagcag aaggatcacc |
| | acaaccctca tctaatcctc |
| 841 | atgatgattc ccccacaccg gctcgacaac ccgggccagg |
| | ggggtcagag gaagaagcgg |
| 901 | gctttggaca ccaattactg cttccggtag |

By “TGFβR1 polypeptide” is meant a polypeptide or fragment thereof having at least about 85% amino acid identity to GenBank Accession No. AAH71181.1 and having a biological activity of a TGFβR1 polypeptide. Biological activities of a TGFβR1 polypeptide include binding to ligands TGFβ 1, TGFβ2, and TGFβ3 polypeptides, and transduction of a signal from TGFβ1, TGFβ2, or TGFβ3 polypeptide binding from the cell surface to the cytoplasm. The sequence at GenBank Accession No. AAH71181.1 is shown below (SEQ ID NO: 31):

| | |
|---|---|
| 1 | meaavaaprp rllllvlaaa aaaaaallpg atalqcfchl |
| | ctkdnftcvt dglcfvsvte |
| 61 | ttdkvihnsm ciaeidlipr drpfvcapss ktgsvtttyc |
| | cnqdhcnkie lpttglpllv |
| 121 | qrtiartivl qesigkgrfg evwrgkwrge evavkifssr |
| | eerswfreae iyqtvmlrhe |
| 181 | nilgfiaadn kdngtwtqlw lvsdyhehgs lfdylnrytv |
| | tvegmiklal stasglahlh |
| 241 | meivgtqgkp aiahrdlksk nilvkkngtc ciadlglavr |
| | hdsatdtidi apnhrvgtkr |
| 301 | ymapevldds inmkhfesfk radiyamglv fweiarrcsi |
| | ggihedyqlp yydlvpsdps |
| 361 | veemrkvvce qklrpnipnr wqscealrvm akimrecwya |
| | ngaarltalr ikktlsqlsq |
| 421 | qegikm |

By “TGFβR1 polynucleotide” is meant a polynucleotide encoding a TGFβR1 polypeptide. An exemplary TGFβR1 polynucleotide sequence is provided at GenBank Accession No. BC071181.1. The exemplary sequence provided at GenBank Accession No. BC071181.1 is reproduced below (SEQ ID NO: 32).

| | |
|---|---|
| 1 | gcggcggcta gggaggtggg gcgaggcgag gtttgctggg gtgaggcagc ggcgcggccg |
| 61 | ggccgggccg ggccacaggc ggtggcggcg ggaccatgga ggcggcggtc gctgctccgc |
| 121 | gtccccggct gctcctcctc gtgctggcgg cggcggcggc ggcggcggcg gcgctgctcc |
| 181 | cgggggcgac ggcgttacag tgtttctgcc acctctgtac aaaagacaat tttacttgtg |
| 241 | tgacagatgg gctctgcttt gtctctgtca cagagaccac agacaaagtt atacacaaca |
| 301 | gcatgtgtat agctgaaatt gacttaattc ctcgagatag gccgtttgta tgtgcaccct |
| 361 | cttcaaaaac tgggtctgtg actacaacat attgctgcaa tcaggaccat tgcaataaaa |
| 421 | tagaacttcc aactactggt ttaccattgc ttgttcagag aacaattgcg agaactattg |
| 481 | tgttacaaga aagcattggc aaaggtcgat ttggagaagt ttggagagga aagtggcggg |
| 541 | gagaagaagt tgctgttaag atattctcct ctagagaaga acgttcgtgg ttccgtgagg |
| 601 | cagagattta tcaaactgta atgttacgtc atgaaaacat cctgggattt atagcagcag |
| 661 | acaataaaga caatggtact tggactcagc tctggttggt gtcagattat catgagcatg |
| 721 | gatccctttt tgattactta aacagataca cagttactgt ggaaggaatg ataaaacttg |
| 781 | ctctgtccac ggcgagcggt cttgcccatc ttcacatgga gattgttggt acccaaggaa |
| 841 | agccagccat tgctcataga gatttgaaat caaagaatat cttggtaaag aagaatggaa |
| 901 | cttgctgtat tgcagactta ggactggcag taagacatga ttcagccaca gataccattg |
| 961 | atattgctcc aaaccacaga gtgggaacaa aaaggtacat ggcccctgaa gttctcgatg |
| 1021 | attccataaa tatgaaacat tttgaatcct tcaaacgtgc tgacatctat gcaatgggct |
| 1081 | tagtattctg ggaaattgct cgacgatgtt ccattggtgg aattcatgaa gattaccaac |
| 1141 | tgccttatta tgatcttgta ccttctgacc catcagttga agaaatgaga aaagttgttt |
| 1201 | gtgaacagaa gttaaggcca aatatcccaa acagatggca gagctgtgaa gccttgagag |
| 1261 | taatggctaa aattatgaga gaatgttggt atgccaatgg agcagctagg cttacagcat |
| 1321 | tgcggattaa gaaaacatta tcgcaactca gtcaacagga aggcatcaaa atgtaattct |
| 1381 | acagctttgc ctgaactctc cttttttctt cagatctgct cctgggtttt aatttgggag |
| 1441 | gtcaattgtt ctacctcact gagagggaac agaaggatat tgcttccttt tgcagcagtg |
| 1501 | taataaagtc aattaaaaac ttcccaggat ttctttggac ccaggaaaca gccatgtggg |
| 1561 | tcctttctgt gcactatgaa cgcttctttc ccaggacaga aaatgtgtag tctaccttta |
| 1621 | ttttttatta acaaaacttg ttttttaaaa agatgattgc tggtcttaac tttaggtaac |
| 1681 | tctgctgtgc tggagatcat ctttaagggc aaaggagttg gattgctgaa ttacaatgaa |
| 1741 | acatgtctta ttactaaaga aagtgattta ctcctggtta gtacattctc agaggattct |
| 1801 | gaaccactag agtttccttg attcagactt tgaatgtact gttctatagt ttttcaggat |
| 1861 | cttaaaacta acacttataa aactcttatc ttgagtctaa aaatgacctc atatagtagt |
| 1921 | gaggaacata attcatgcaa ttgtattttg tatactatta ttgttctttc acttattcag |
| 1981 | aacattacat gccttcaaaa tgggattgta ctataccagt aagtgccact tctgtgtctt |
| 2041 | tctaatggaa atgagtagaa ttgctgaaag tctctatgtt aaaacctata gtgtttgaat |
| 2101 | tcaaaaagct tatttatctg ggtaacccaa actttttctg ttttgttttt ggaagggttt |
| 2161 | ttgtggtatg tcatttggta ttctattctg aaaatgcctt tctcctacca aaatgtgctt |
| 2221 | aagccactaa agaaatgaag tggcattaat tagtaaatta ttagcatggt catgtttgaa |
| 2281 | tattctcaca tcaagctttt gcattttaat tgtgttgtct aagtatactt ttaaaaaatc |
| 2341 | aagtggcact ctagatgctt atagtacttt aatatttgta gcatacagac taatttttct |
| 2401 | aaaagggaaa gtctgtctag ctgcttgtga aaagttatgt ggtattctgt aagccatttt |
| 2461 | tttctttatc tgttcaaaga cttatttttt aagacatgaa ttacatttaa aattagaata |
| 2521 | tggttaatat taaataatag gcctttttct aggaaggcga aggtagttaa taatttgaat |
| 2581 | agataacaga tgtgcaagaa agtcacattt gttatgtatg taggagtaaa cgttcggtgg |
| 2641 | atcctctgtc tttgtaactg aggttagagc tagtgtggtt ttgaggtctc actacacttt |
| 2701 | gaggaaggca gcttttaatt cagtgtttcc ttatgtgtgc gtacattgca actgcttaca |
| 2761 | tgtaatttat gtaatgcatt cagtgcaccc ttgttacttg ggagaggtgg tagctaaaga |
| 2821 | acattctgag tataggtttt tctccattta cagatgtctt tggtcaaata ttgaaagcaa |
| 2881 | acttgtcatg gtcttcttac attaagttga aactagctta taataactgg tttttacttc |
| 2941 | caatgctatg aagtctctgc agggctttta cagttttcga agtcctttta tcactgtgat |
| 3001 | cttattctga ggggagaaaa aactatcata gctctgaggc aagacttcga ctttatagtg |
| 3061 | ctatcagttc cccgatacag ggtcagagta acccatacag tattttggtc aggaagagaa |
| 3121 | agtggccatt tacactgaat gagttgcatt ctgataatgt cttatctctt atacgtagaa |
| 3181 | taaatttgaa agactatttg atcttaaaac caaagtaatt ttagaatgag tgacatatta |
| 3241 | cataggaatt tagtgtcaat ttcatgtgtt taaaaacatc atgggaaaaa tgcttagagg |
| 3301 | ttactatttt gactacaaag ttgagttttt ttctgtagtt accataattt cattgaagca |
| 3361 | aatgaatgag tttgagaggt ttgtttttat agttgtgttg tattacttgt ttaataataa |
| 3421 | tctctaattc tgtgatcagg tacttttttt gtgggggttt tttttttgtt tttttttttt |
| 3481 | tttgttgttg tttttgggcc atttctaagc ctaccagatc tgctttatga aatccagggg |
| 3541 | accaatgcat tttatcacta aaactatttt tatataattt taagaatata ccaaaagttg |
| 3601 | tctgatttaa agttgtaata catgatttct cactttcatg taaggttatc cacttttgct |
| 3661 | gaagatattt tttattgaat caaagattga gttacaatta tacttttctt acctaagtgg |
| 3721 | ataaaatgta cttttgatga atcagggaat ttttttaaag ttggagttta gttctaaatt |
| 3781 | gactttacgt attactgcag ttaattcctt ttttggctag ggatggtttg ataaaccaca |
| 3841 | attggctgat attgaaaatg aaagaaactt aaaaggtggg atggatcatg attactgtcg |
| 3901 | ataactgcag ataaatttga ttagagtaat aattttgtca tttaaaaaca cagttgttta |
| 3961 | tactgcccat cctaggatgc tcaccttcca agattcaacg tggctaaaac atcttctggt |
| 4021 | aaattgtgcg tccatattca ttttgtcagt agccaggaga aatggggatg ggggaaatac |
| 4081 | gacttagtga ggcatagaca tccctggtcc atcctttctg tctccagctg tttcttggaa |
| 4141 | cctgctctcc tgcttgctgg tccctgacgc agagaccgtt gcctccccca cagccgtttg |
| 4201 | actgaaggct gctctggaga cctagagtaa aacggctgat ggaagttgtg ggacccactt |
| 4261 | ccatttcctt cagtcattag aggtggaagg gaggggtctc caagtttgga gattgagcag |
| 4321 | atgaggcttg ggatgcccct gctttgactt cagccatgga tgaggagtgg gatggcagca |
| 4381 | aggtggctcc tgtggcagtg gagttgtgcc agaaacagtg gccagttgta tcgcctataa |
| 4441 | gacagggtaa ggtctgaaga gctgagcctg taattctgct gtaataatga tagtgctcaa |
| 4501 | gaagtgcctt gagttggtgt acagtgccat ggccatcaag aatcccagat ttcaggtttt |
| 4561 | attacaaaat gtaagtggtc acttggcgat tttgtagtac atgcatgagt tacctttttt |
| 4621 | ctctatgtct gagaactgtc agattaaaac aagatggcaa agagatcgtt agagtgcaca |
| 4681 | acaaaatcac tatcccatta gacacatcat caaaagctta tttttattct tgcactggaa |
| 4741 | gaatcgtaag tcaactgttt cttgaccatg gcagtgttct ggctccaaat ggtagtgatt |
| 4801 | ccaaataatg gttctgttaa cactttggca gaaaatgcca gctcagatat tttgagatac |
| 4861 | taaggattat ctttggacat gtactgcagc ttcttgtctc tgttttggat tactggaata |
| 4921 | cccatgggcc ctctcaagag tgctggactt ctaggacatt aagatgattg tcagtacatt |
| 4981 | aaacttttca atcccattat gcaatcttgt ttgtaaatgt aaacttctaa aaatatggtt |
| 5041 | aataacattc aacctgttta ttacaactta aaaggaactt cagtgaattt gtttttattt |
| 5101 | tttaacaaga tttgtgaact gaatatcatg aaccatgttt tgatacccct ttttcacgtt |
| 5161 | gtgccaacgg aatagggtgt ttgatatttc ttcatatgtt aaggagatgc ttcaaaatgt |
| 5221 | caattgcttt aaacttaaat tacctctcaa gagaccaagg tacatttacc tcattgtgta |
| 5281 | tataatgttt aatatttgtc agagcattct ccaggtttgc agttttattt ctataaagta |
| 5341 | tgggtattat gttgctcagt tactcaaatg gtactgtatt gtttatattt gtaccccaaa |
| 5401 | taacatcgtc tgtactttct gttttctgta ttgtatttgt gcaggattct ttaggcttta |
| 5461 | tcagtgtaat ttctgccttt taagatatgt acagaaaatg tccatataaa tttccattga |
| 5521 | agtcgaatga tactgagaag cctgtaaaga ggagaaaaaa cataagctgt gtttccccat |
| 5581 | aagttttttt aaattgtata ttgtatttgt agtaatattc caaaagaatg taaataggaa |
| 5641 | atagaagagt gatgcttatg ttaagtccta acactacagt agaagaatgg aagcagtgca |
| 5701 | aataaattac atttttccca aaaaaaaaaa aaaaaaaaaa aaaaaaaaaa gaaaaaaaaa |
| 5761 | aaaaaa |

By “TGFβR2 polypeptide” is meant a polypeptide or fragment thereof having at least about 85% amino acid identity to GenBank Accession No. ABG65632.1 and having a biological activity of a TGFβR2 polypeptide. Biological activities of a TGFβR2 polypeptide include binding to TGFβR1 polypeptide to form a heterodimeric complex, and serine/threonine kinase activity. The sequence at GenBank Accession No. ABG65632.1 is shown below (SEQ ID NO: 33):

| | |
|---|---|
| 1 | mgrgllrglw plhivlwtri astipphvqk svnndmivtd |
| | nngavkfpql ckfcdvrfst |
| 61 | cdnqkscmsn csitsicekp qevcvavwrk ndenitletv |
| | chdpklpyhd filedaaspk |
| 121 | cimkekkkpg etffmcscss decndniifs eeyntsnpdl |
| | llvifqvtgi sllpplgvai |
| 181 | sviiifycyr vnrqqklsst wetgktrklm efsehcaiil |
| | eddrsdisst canninhnte |
| 241 | llpieldtlv gkgrfaevyk aklkqntseq fetvavkifp |
| | yeeyaswkte kdifsdinik |
| 301 | henilqflta eerktelgkq ywlitafhak gnlqeyltrh |
| | viswedlrkl gsslargiah |
| 361 | ihsdhtpcgr pkmpivhrdl kssnilvknd ltcclcdfgl |
| | slrldptlsv ddlansgqvg |
| 421 | tarymapevl esrmnlenve sfkqtdvysm alvlwemtsr |
| | cnavgevkdy eppfgskvre |
| 481 | hpcvesmkdn vlrdrgrpei psfwlnhqgi qmvcetltec |
| | wdhdpearlt aqcvaerfse |
| 541 | lehldrlsgr scseekiped gslnttk |

By “TGFβR2 polynucleotide” is meant a polynucleotide encoding a TGFβR2 polypeptide. An exemplary TGFβR2 polynucleotide sequence is provided at GenBank Accession No. DQ377553.1. The exemplary sequence provided at GenBank Accession No. DQ377553.1 is reproduced below (SEQ ID NO: 34).

CCTCCTGGCTGGCGAGCGGGCGCCACATCTGGCCCGCACATCTGCGCTGC
CGGCCCGGCGCGGGGTCCGGAGAGGGCGCGGCGCGGAGGCGCAGCCAGGG
GTCCGGGAAGGCGCCGTCCGCTGCGCTGGGGGCTCGGTCTATGACGAGCA
GCGGGGTCTGCCATGGGTCGGGGGCTGCTCAGGGGCCTGTGGCCGCTGCA
CATCGTCCTGTGGACGCGTATCGCCAGCACGATCCCACCGCACGTTCAGA
AGTCGGGTGAGTGGTCCCCAGCCCGGGCTCGGCGGGGCGCCGGGGGTCTT
CCTGGGGTCCCCGCCTCTCCGCTGCGCTTGACAGTCGGGCCCGGCAACCC
GGCCCCCGGGCGGAAACGAGGAAAGTTTCCCCCGCGACACTCACGCAGCC
CGACTCCCGTAGCTGCAGGGATTGTGAGTTTTTCTTGAAAAAGAGAAGGA
AAGTTCAGTTGCAAGGGGCGCGGGGCACGTTTGGTCC

As used herein, the term “rapamycin” refers to a compound (a macrocyclic triene antibiotic also known as Sirolimus) produced by the bacterium Streptomyces hygroscopicus. It inhibits the activation of T cells and B cells by reducing the production of interleukin-2 (IL-2). Rapamycin has immunosuppressant functions in humans and is especially useful in medicine for preventing organ transplant rejection such as the rejection of kidney transplants. It is also used to treat lymphangioleiomyomatosis, a lung progressive and systemic disease. Rapamycin has also been shown to inhibit proliferation of vascular smooth muscle cells migration (Poon M. et al., J Clin Invest. 1996; 98(10):2277-83). Rapamycin derivatives used according to the methods of present invention include, but are not limited to, 40-0-alkyl-rapamycin derivatives, e.g. 40-O-hydroxyalkyl-rapamycin derivatives, for example 40-O-(2-hydroxy)-ethyl-rapamycin (everolimus), rapamycin derivatives which are substituted in 40 position by heterocyclyl, e.g. 40-epi-(tetrazolyi)-rapamycin (also known as ABT578), 32-deoxo-rapamycin derivatives and 32-hydroxy-rapamycin derivatives, such as 32-deoxorapamycin, 16-O-substituted rapamycin derivatives such as 16-pent-2-ynyloxy-32-deoxorapamycin, 16-pent-2-ynyloxy-32(S or R)-dihydro-rapamycin, or 16-pent-2-ynyloxy-32(S or R)-dihydro-40-O-(2-hydroxyethyl)-rapamycin, rapamycin derivatives which are acylated at the oxygen in position 40, e.g. 40-[3-hydroxy-2-(hydroxy-methyl)-2-methylpropanoate]-rapamycin (also known as CCI779 or temsirolimus), rapamycin derivatives as disclosed in WO9802441 or WO0114387 (also sometimes designated as rapalogs), e.g. including AP23573, such as 40-O-dimethylphosphinyl-rapamycin, compounds disclosed under the name biolimus (biolimus A9), including 40-O-(2-ethoxy)ethyl-rapamycin, and compounds disclosed under the name TAFA-93, AP23464, AP23675 or AP23841; or rapamycin derivatives as e.g. disclosed in WO2004101583, WO9205179, WO9402136, WO9402385 and WO9613273.

By “subject” is meant a mammal, including, but not limited to, a human or non-human mammal, such as a bovine, equine, canine, ovine, murine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION

Molecular events that drive progression of atherosclerosis Atherosclerosis is responsible for the vast majority of cardiovascular diseases. Despite decades of work, statins remain the only effective therapy but they can only slow but not stop or reverse disease progression. The relentless nature of atherosclerosis implies the existence of a process that drives its progression, even if the agents responsible for its initiation have been removed. Hyperlipidemia, local disturbances in fluid shear stress, smoking, hypertension etc., induce an initial vascular inflammatory response in the vessel wall characterized by the presence of macrophages, leukocytes, and production of the fibronectin-rich matrix. The disease becomes progressive as the initial bout of inflammation induces an endothelial fate change that leads to the development of endothelial-to-mesenchymal transition (EndMT). The EndMT is a process that involves phenotypic change and migration of epithelial cells into the sub-epithelial mesenchyme in the lamina propria (LP) that function as extracellular-matrix producing fibroblasts/myofibroblasts. EndMT is a vital process during embryogenesis, but can also be induced as a result of persistent damage and tissue inflammation. Active EndMT can lead to severe and even complete organ fibrosis or development of a pre-malignant stroma when associated with angiogenesis.

EndMT not only drives the accumulation of “mesenchymal type” (smooth muscle, fibroblasts) cells in the plaque, but induces further inflammatory activation of luminal endothelial cells, extracellular matrix remodeling, and increased permeability. These events promote further entry and retention of both leukocytes and lipoproteins, which promote further inflammation and further EndMT, thereby creating a self-sustaining feed-forward loop. Once set in motion, this process continues even if initiating factors are no longer present. Described herein are methods to arrest atherosclerosis and induce regression of the established disease by inhibiting EndMT using a therapeutic strategy applicable to large numbers of patients.

EndMT occurs in various inflammatory conditions. EndMT plays an equally important role in transplant arteriosclerosis, a relentless disease that is the primary reason for long-term failure of various organ drugs, such as for the heart or kidneys. There are no known therapies for this condition. EndMT is also important in pulmonary hypertension and various conditions associated with chronic inflammation induced fibrosis such as scleroderma, Systemic Lupus Erythematosus (SLE), transplant arteriopathy, cystitic fibrosis and other fibrosis and the like to name a few. Accordingly, without being bound by theory, the same treatment that is effective in reducing atherosclerosis is expected to be effective in treatment of the foregoing diseases.

In addition to EndMT, another major driver of long-term plaque growth is the loss of media smooth muscle cell (SMC) differentiation leading to uncontrolled proliferation. Described herein is the discovery of a molecular pathway controlling this process and a demonstration, using mouse genetics, that upregulating it reduced plaque size by ˜50%. Without intending to be bound by theory, combining the endothelial approach outlined above with SMC-targeted therapy has the high likelihood of completely blocking atherosclerosis development and progression.

The conversion of vascular smooth muscle cells (SMCs) from contractile to proliferative phenotype is thought to play an important role in atherosclerosis. However, the contribution of this process to plaque growth has never been fully defined. The study described herein reveals that activation of SMC TGFβ signaling, achieved by suppression of SMC FGF signaling input, induces their conversion to a contractile phenotype and dramatically reduces atherosclerotic plaque size. The FGF-TGFβ signaling cross-talk was observed in vitro and in vivo. In vitro, inhibition of FGF signaling increased TGFβ activity thereby promoting smooth muscle differentiation and decreasing proliferation. In vivo, smooth muscle-specific knockout of an FGF receptor adaptor Frs2α led to a profound inhibition of atherosclerotic plaque growth when these animals were crossed on Apoe^{-/-} background and subjected to a high fat diet. In particular, there was a significant reduction in plaque cellularity, increase in fibrous cap area and decrease in necrotic core size. In agreement with these findings, examination of human coronary arteries with various degrees of atherosclerosis revealed a strong correlation between the activation of FGF signaling, loss of TGFβ activity, and increased disease severity. These results identify SMC FGF/TGFβ signaling cross-talk as an important regulator of SMC phenotype switch and document a major contribution of medial SMC proliferation to atherosclerotic plaque growth.

### Therapeutic Strategy for Inhibiting or Reversing Atherosclerosis

Described herein are studies demonstrating the key role of FGF signaling, let-7 miRNA expression, and TGFβ signaling in the progression of atherosclerosis by induction of endothelial-to-mesenchymal transition (EndMT) in endothelial cells and by promotion of a proliferative phenotype in smooth muscle cells. In endothelial cells, overexpression of let-7 miRNA and consequently decreased TGFβ signaling reduced atherosclerotic lesions. In smooth muscle cells, disruption of FGF signaling by deletion of FRS2α, which reduced let-7 miRNA expression and increased TGFβ signaling, was found to reduce atherosclerotic lesions. This is summarized in schematic form in FIG. 8.

Provided herein are methods to arrest atherosclerosis and induce regression of the established disease by inhibiting EndMT or smooth muscle cell proliferation using a therapeutic strategy applicable to large numbers of patients. Currently, there is no available therapy to stop the development of atherosclerosis and induce its regression. Described herein is a key mechanism responsible for atherosclerosis progression and studies demonstrating that modulating this pathway fundamentally changes the natural history or course of the disease. The mechanism involves a link between FGF signaling, let-7 miRNA, and TGFβ signaling. Targeting this mechanism would dramatically alter the management of atherosclerosis and would represent a major practical breakthrough.

The therapeutic approach described herein, based on insights derived from cell signaling studies and confirmed by rigorous in vivo mouse genetics studies and human data, is fundamentally new. Instead of trying to limit the disease complications, the focus of current approaches, the present invention includes a therapeutic approach that alters the biology of the cell type that initiates and sustains atherosclerosis in order to arrest and reverse the process. The highly targeted nature of the approach, the genetic proof of principle that this strategy works, and the ability to specifically target endothelium in a manner suitable to widespread clinical applications, renders the invention highly useful for treatment of atherosclerosis.

### Endothelial-to-Mesenchymal Transition

The endothelial-to-mesenchymal transition (EndMT) is induced by activation of endothelial TGFβ signaling that occurs secondary to the loss of a protective FGF input. In healthy vessels, FGF suppresses TGFβ signaling by inducing the let-7 family of miRNAs that reduce expression of key TGFβ pathway proteins (TGFβ2, TGFβR1, Smad2). The importance of the FGF-let-7-TGFβ link is supported by human and mouse data. In human coronary arteries, a strong correlation between the reduction in FGFR1 expression, increase in p-Smad2/3 signaling, and the extent of atherosclerosis (r=0.84, p<0.01) was observed (Chen et al, 2015, Journal of clinical investigation 125: 4529-4543). Described herein is a demonstration that in mice, blocking TGFβ signaling by endothelial-specific deletion of TGFβR1 and TGFβR2, dramatically reduces atherosclerosis.

Thus, in some embodiments, the TGFβ signaling is blocked by delivering let-7 miRNA into a cell. In a particular embodiment, the cell is an endothelial cell. In a particular embodiment, a systemic treatment strategy using a modified let-7 miRNA delivered to endothelial cells in targeted nanoparticles is employed. In some embodiments, the modified let-7 miRNA is mi-let-7b_{L} or mi-let-7b_{H}. Studies in mice demonstrate that this approach is as effective in reducing atherosclerosis as a TGFβR1/2 knockout.

In some embodiments, the therapy is cell-type specific. Systemic inhibition of TGFβ signaling has an adverse effect on atherosclerosis by promoting inflammation and smooth muscle cell proliferation.

In some embodiments, TGFβR1/2 targeted siRNAs are delivered to endothelial cells. In some embodiments, TGFβR1/2 targeted siRNAs therapy is as effective as let-7-based therapy for reducing atherosclerosis.

Described herein is genetic proof of the proposed therapeutic strategy, evidence of its clinical relevance, and the development of an effective systemic therapeutic approach suitable for large numbers of patients. Further provided herein is evidence that there are specific FGF-dependent metabolic controls that can be used to block EndMT.

In some embodiments, the invention provides a method of reducing, inhibiting or reversing an EndMT in an endothelial cell in a subject in need thereof. The method comprises administering to the subject an agent that decreases in the endothelial cell of the subject the activity or level of at least one selected from the group consisting of let-7 miRNA, endothelial TGFβ signaling polypeptide and FRS2α, thereby reducing, inhibiting or reversing the EndMT in the endothelial cell in the subject in need thereof.

Without intending to be bound by theory, it is believed that a combination of these strategies, aimed at interrupting the EndMT/inflammation cycle, provides a definitive therapeutic approach to atherosclerosis.

### Smooth Muscle Cell Proliferative-to-Contractile Phenotype Switching

In the studies described herein, it was hypothesized that suppression of FGF signaling in SMC would induce a contractile phenotype and that this enforced maintenance of contractile SMC phenotype would diminish any contributions of media smooth muscle cells proliferation to atherosclerotic plaque growth. To investigate this hypothesis, a mouse line with an SMC-specific deletion of a key FGF signaling regulator Frs2α was generated. The shutdown of FGF-induced MAPK signaling in SMCs induced by Frs2α knockout resulted in increased expression of TGFβ ligands and receptors and activation of TGFβ signaling. In vitro this led to a growth arrest of proliferating SMCs and induction of their differentiation while in vivo there was a profound reduction in the size of atherosclerotic lesions. Analysis of clinical specimens confirmed the inverse relationship between the extent of medial FGF and TGFβ signaling and the severity of atherosclerosis. Overall, the results herein demonstrate that FGF regulates SMC phenotypic modulation by controlling SMC TGFβ signaling and directly elucidate the contribution of SMC proliferation to the growth of atherosclerotic plaque.

Accordingly, in some embodiments, the TGFβ signaling is activated by delivering to a cell an inhibitory polynucleotide that reduces SMC expression of FRS2α polypeptide or reduces SMC expression of a let-7 miRNA. In some embodiments, the TGFβ signaling is activated by delivering to an SMC an agent that increases the activity or level of a TGFβ signaling polypeptide. In a particular embodiment, the cell is an smooth muscle cell.

### Methods of Treatment

In some aspects, the present invention provides a method of treating atherosclerosis and/or disorders or symptoms thereof which comprise administering a therapeutically effective amount of a pharmaceutical composition comprising an agent that modulates the activity or level of a TGFβ signaling polypeptide, a let-7 miRNA, or a FGF signaling polypeptide in a cell, to a subject (e.g., a mammal such as a human).

In particular embodiments, the agent that modulates the activity or level of a let-7 miRNA increases the activity or level of a let-7 miRNA in a cell. In some embodiments, the cell is an endothelial cell. In certain embodiments, the agent that increases the activity or level of a let-7 miRNA in a cell is a let-7 miRNA mimic. In some other embodiments, the agent is a polynucleotide encoding a let-7b miRNA. In some embodiments, the let-7 miRNA is let-7b and let-7c miRNA.

In some embodiments, the agent that modulates the activity or level of a let-7 miRNA decreases the activity or level of a let-7 miRNA in a cell. In certain embodiments, the cell is a smooth muscle cell. In some embodiments, the agent that decreases the activity or level of a let-7 miRNA in a cell is an inhibitory polynucleotide that reduces expression of let-7 miRNA.

In still other embodiments, the agent that decreases the activity or level of a let-7 miRNA in a cell is a let-7 miRNA sponge or antagomir-let-7b/c. Such miRNA sponges are described in, for example, Ebert et al. RNA. 2010 Nov.; 16(11): 2043-2050. In some embodiments, the let-7 miRNA is let-7b miRNA.

In some embodiments, the agent that modulates the activity or level of a TGFβ signaling polypeptide increases the activity or level of a TGFβ signaling polypeptide in a cell (in particular, a smooth muscle cell). In some other embodiments, the agent that modulates the activity or level of a TGFβ signaling polypeptide decreases the activity or level of a TGFβ signaling polypeptide in a cell (in particular, an endothelial cell). In some embodiments, the TGFβ signaling polypeptide is TGFβ 1, TGFβ2, TGFβ3, TGFβR1, or TGFβR2. In some embodiments, the agent is siRNA and may be targeted to a TGFβ receptor.

In some embodiments, the agent that decreases the activity or level of a TGFβ signaling polypeptide is an inhibitory polynucleotide that reduces expression of a TGFβ signaling polypeptide. In some other embodiments, the agent that increases the activity or level of a TGFβ signaling polypeptide is a polynucleotide encoding a TGFβ signaling polypeptide.

In certain embodiments, the agent that modulates the activity or level of a FGF signaling polypeptide decreases the activity or level of a FGF signaling polypeptide in a cell (in particular, a smooth muscle cell). In some embodiments, the agent that modulates the activity or level of a FGF signaling polypeptide increases the activity or level of a FGF signaling polypeptide in a cell (in particular, an endothelial cell). In some embodiments, the FGF signaling polypeptide is FRS2α.

In certain embodiments, the agent that decreases the activity or level of a FGF signaling polypeptide in a cell is an inhibitory polynucleotide that reduces expression of a FGF signaling polypeptide. In some other embodiments, the agent that increases the activity or level of a FGF signaling polypeptide in a cell is a polynucleotide encoding a FGF signaling polypeptide.

In some embodiments, the subject is pre-selected by assessing the activity or level of a TGFβ signaling polypeptide or polynucleotide, a let-7 miRNA, or a FGF signaling polypeptide or polynucleotide in a sample from the subject when compared to reference levels.

The subject is pre-selected when an alteration in the activity or level of activity or level of a TGFβ signaling polypeptide or polynucleotide, a let-7 miRNA, or a FGF signaling polypeptide or polynucleotide in a sample from the subject is detected. In some embodiments, the subject is pre-selected when a decrease in the activity or level of let-7 miRNA or a TGFβ signaling polypeptide is observed relative to reference levels in an endothelial cell sample obtained from the subject. In other embodiments, the subject is pre-selected when a decrease in the activity or level of a FGF signaling polypeptide or polynucleotide, or an increase in the activity or level of let-7 miRNA or a TGFβ signaling polypeptide or polynucleotide is observed relative to reference levels in a smooth muscle cell sample obtained from the subject.

In other embodiments, the subject is pre-identified as having or being at risk for atherosclerosis, in certain embodiments patients suffering from coronary artery disease (CAD), peripheral vascular disease (PVD), or stroke. In other embodiments, the patient may have one or more known atherosclerotic plaques or may have experienced one or more recent ischemic events, in certain embodiments, transient ischemic attack (TIA), unstable angina (UA), or myocardial infarction (MI). In other embodiments, the subject has elevated cholesterol and/or a history of PVD, CAD or other cardiovascular disease. Thus, in one embodiment, there is provided a method of treating a subject suffering from or susceptible to atherosclerosis or disorder or symptom thereof. The method includes the step of administering to the mammal a therapeutic amount of an agent (e.g., an agent that modulates the activity or level of a TGFβ signaling polypeptide, a let-7 miRNA, or a FGF signaling polypeptide) that is sufficient to treat the disease or disorder or symptom thereof, under conditions such that the disease or disorder is treated.

In some aspects of the invention, the subject is administered an additional agent comprising a therapeutically effective amount of rapamycin or any derivative thereof. In some embodiments, the therapeutically effective amount of rapamycin or any derivative thereof is used to reduce SMC proliferation and increase its differentiation alone or in combination with EC-specific therapies. In some embodiments, the agent that decreases the activity or level of a TGFβ signaling polypeptide and the additional agent are co-administered to the subject.

In other aspects of the invention, the agent that decreases the activity or level of a TGFβ signaling polypeptide is a nucleic acid capable of downregulating the gene expression of at least one gene selected from the group consisting of TGFβ 1, TGFβ2, TGFβ3, TGFβR1, and TGFβR2. In some embodiments, the at least one gene is selected from the group consisting of TGFβR1, and TGFβR2.

In some instance, downregulation of the TGFβ or TGFβ receptor (TGFβR) gene expression is desired. This downregulation may result from a full or partial knock down of the gene of interest. Briefly, a gene knock down refers to a genetic technique in which one of an organism's genes is silenced, made inoperative or partially inoperative. Gene expression may be downregulated, knocked-down, decreased, and/or inhibited by various well-established molecular techniques known in the art such as, but not limited to, RNA interference (RNAi), small inhibitor RNA (siRNA), small hairpin RNA (shRNA) and Clustered Regularly Interspaced Short Palindromic Repeats (CRISPRs)).

In some embodiments, the nucleic acid is selected from the group consisting of an antisense RNA, siRNA, shRNA, and a CRISPR system. In other embodiments, the nucleic acid is combined with a therapeutically effective amount of rapamycin or any derivative thereof. In yet other embodiments, the nucleic acid is encapsulated in a nanoparticle formulated for selective delivery to an endothelial cell, in a pharmaceutically acceptable excipient. In further embodiments, the nanoparticle is a 7C1 nanoparticle.

The methods disclosed herein include administering to the subject (including a subject identified as in need of such treatment) an effective amount of an agent described herein, or a composition described herein to produce such effect. Identifying a subject in need of such treatment can be made by a health care professional and may be subjective (e.g. opinion) or objective (e.g. measurable by a test or diagnostic method, such as using the methods described herein).

The therapeutic methods of the invention, which may also include prophylactic treatment, in general comprise administering a therapeutically effective amount of one or more of the agents herein (such as an agent that modulates the activity or level of a TGFβ signaling polypeptide, a let-7 miRNA, or a FGF signaling polypeptide) to a subject (e.g., animal, human) in need thereof, including a mammal, particularly a human. Such treatment is suitable for subjects, particularly humans, suffering from, having, susceptible to, or at risk for a atherosclerosis, disorder, or symptom thereof. In one embodiment, the invention provides a method of monitoring progression of treatment. The method comprises determining a level or activity of diagnostic marker (e.g., a TGFβ signaling polypeptide or polynucleotide, a let-7 miRNA, or a FGF signaling polypeptide or polynucleotide) in a subject suffering from or susceptible to a atherosclerosis, in which the subject has been administered a therapeutic or effective amount of a therapeutic agent sufficient to treat the atherosclerosis or symptoms thereof. The activity or level of a TGFβ signaling polypeptide or polynucleotide, a let-7 miRNA, or a FGF signaling polypeptide or polynucleotide determined in the method can be compared to a known activity or level of a TGFβ signaling polypeptide or polynucleotide, a let-7 miRNA, or a FGF signaling polypeptide or polynucleotide in either healthy normal controls, or in other afflicted patients, to establish the subject's disease status. In some embodiments, an activity or level of a TGFβ signaling polypeptide or polynucleotide, a let-7 miRNA, or a FGF signaling polypeptide or polynucleotide in an endothelial cell or smooth muscle cell sample obtained from the subject is determined. In some embodiments, a second activity or level of a TGFβ signaling polypeptide or polynucleotide, a let-7 miRNA, or a FGF signaling polypeptide or polynucleotide in the subject is determined at a time point later than the determination of the first level, and the two levels are compared to monitor the course of disease or the efficacy of the therapy. In certain embodiments, a pre-treatment activity or level of a TGFβ signaling polypeptide or polynucleotide, a let-7 miRNA, or a FGF signaling polypeptide or polynucleotide is determined prior to commencing. This pre-treatment level can then be compared to the level of a TGFβ signaling polynucleotide or polypeptide or let-7 miRNA in the subject after the treatment commences, to determine the progress or efficacy of the treatment.

### Pharmaceutical Compositions

The present invention features compositions useful for treating atherosclerosis in a pre-selected subject. The compositions include an agent that modulates the activity or level of a TGFβ signaling polypeptide, a let-7 miRNA, or a FGF signaling polypeptide in a cell.

In particular embodiments, the agent that modulates the activity or level of a let-7 miRNA increases the activity or level of a let-7 miRNA in a cell, in particular, an endothelial cell. In certain embodiments, the agent that increases the activity or level of a let-7 miRNA in a cell is a let-7 miRNA mimic. In some other embodiments, the agent is a polynucleotide encoding a let-7b miRNA. In certain embodiments, the agent that modulates the activity or level of a let-7 miRNA decreases the activity or level of a let-7 miRNA in a cell, in particular, a smooth muscle cell. In some embodiments, the agent that decreases the activity or level of a let-7 miRNA in a cell is an inhibitory polynucleotide that reduces expression of let-7 miRNA. In some embodiments, the let-7 miRNA is let-7b miRNA.

In some embodiments, the agent that modulates the activity or level of a TGFβ signaling polypeptide increases the activity or level of a TGFβ signaling polypeptide in a cell (in particular, a smooth muscle cell). In some other embodiments, the agent that modulates the activity or level of a TGFβ signaling polypeptide decreases the activity or level of a TGFβ signaling polypeptide in a cell (in particular, an endothelial cell). In some embodiments, the TGFβ signaling polypeptide is TGFβ 1, TGFβ2, TGFβ3, TGFβR1, or TGFβR2.

In some embodiments, the agent that decreases the activity or level of a TGFβ signaling polypeptide is an inhibitory polynucleotide that reduces expression of a TGFβ signaling polypeptide. In some other embodiments, the agent that increases the activity or level of a TGFβ signaling polypeptide is a polynucleotide encoding a TGFβ signaling polypeptide.

In certain embodiments, the agent that modulates the activity or level of a FGF signaling polypeptide decreases the activity or level of a FGF signaling polypeptide in a cell (in particular, a smooth muscle cell). In some embodiments, the agent that modulates the activity or level of a FGF signaling polypeptide increases the activity or level of a FGF signaling polypeptide in a cell (in particular, an endothelial cell). In some embodiments, the FGF signaling polypeptide is FRS2α.

In certain embodiments, the agent that decreases the activity or level of a FGF signaling polypeptide in a cell is an inhibitory polynucleotide that reduces expression of a FGF signaling polypeptide. In some other embodiments, the agent that increases the activity or level of a FGF signaling polypeptide in a cell is a polynucleotide encoding an FGF signaling polypeptide

The composition may be administered systemically, for example, formulated in a pharmaceutically-acceptable buffer such as physiological saline. Routes of administration include, for example, subcutaneous, intravenous, intraperitoneally, intramuscular, or intradermal injections that provide continuous, sustained levels of the agent in the patient.

The amount of the therapeutic agent to be administered varies depending upon the manner of administration, the age and body weight of the patient, and with the clinical symptoms of atherosclerosis. Generally, amounts will be in the range of those used for other agents used in the treatment of atherosclerosis, although in certain instances lower amounts will be needed because of the increased specificity of the agent. A composition is administered at a dosage that decreases effects or symptoms of atherosclerosis as determined by a method known to one skilled in the art.

The therapeutic agent may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target atherosclerosis using carriers or chemical derivatives to deliver the therapeutic agent to a particular cell type (e.g., endothelial cells or smooth muscle cells). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The pharmaceutical composition of this invention could be coated or comprised in a drug-eluting stent (DES) ((Nikam et al., 2014 Med Devices 7:165-78)) that releases at a given site (such as an artery) and pace (i.e. slow release) the composition of this invention.

The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates atherosclerosis, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

In some embodiments, the composition of this invention is delivered locally from, but not limited to, the strut of a stent, a stent graft, a stent cover or a stent sheath. In some embodiments, the composition of this invention comprises a rapamycin or a derivative thereof (e.g. as described in U.S. Pat. No. 6,273,913 B1, incorporated herein by reference).

In some embodiments, the composition comprising the active therapeutic is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Polynucleotide Therapy

In some embodiments, the invention includes a method for treating, slowing the progression of, or reversing atherosclerosis, where a therapeutic polynucleotide activity or level of a TGFβ signaling polypeptide, a let-7 miRNA, or a FGF signaling polypeptide is administered to the subject. In certain embodiments, the polynucleotide is a let-7 miRNA mimic; a polynucleotide encoding let-7 miRNA, a TGFβ signaling polypeptide, or FGF signaling polypeptide; or an inhibitory polynucleotide that reduces expression of a FGF signaling polypeptide, a let-7 miRNA, or a TGFβ signaling polypeptide. Inhibitory polynucleotides include, but are not limited to siRNAs that target a polynucleotide encoding a TGFβ signaling polypeptide, a let-7 miRNA, or a FGF signaling polypeptide.

In particular embodiments, the polynucleotide therapy comprises a let-7 miRNA, a polynucleotide encoding a let-7 miRNA, or an inhibitory polynucleotide that reduces expression of a TGFβ signaling polypeptide. As described elsewhere herein, let-7 miRNA inhibits expression of TGFβ signaling polypeptide(s) in endothelial cells, thereby suppressing TGFβ signaling that drives growth or formation of atherosclerotic lesions.

Such therapeutic polynucleotides can be delivered to cells of a subject having atherosclerosis. The nucleic acid molecules are delivered to the cells of a subject in a form by which they are taken up by the cells so that therapeutically effective levels of the inhibitory nucleic acid molecules are contained within the cells.

Introduction of nucleic acids into cells may be accomplished using any number of methods available in the art. For example, transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, an inhibitory nucleic acid or miRNA (or a precursor to the miRNA) as described can be cloned into a retroviral vector where expression can be driven from its endogenous promoter, from the retroviral long terminal repeat, or from a promoter specific for a target cell type of interest. In some embodiments, the target cell type of interest is an endothelial cell. Other viral vectors that can be used to introduce nucleic acids into cells include, but are not limited to, vaccinia virus, bovine papilloma virus, or herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer a polynucleotide encoding inhibitory nucleic acid molecules that inhibit expression of TGFβ signaling polypeptide.

Non-viral approaches can also be employed for the introduction of the therapeutic to a cell of a patient requiring treatment of atherosclerosis. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In some embodiments, the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of polynucleotide encoding inhibitory nucleic acid molecules into the affected tissues of a patient can also be accomplished by transferring a polynucleotide encoding the inhibitory nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some embodiments, the therapeutic polynucleotide is selectively targeted to an endothelial cell. In some other embodiments, the therapeutic polynucleotide is expressed in an endothelial cell using a lentiviral vector. In still other embodiments, the therapeutic polynucleotide is administered intravenously. In some embodiments, the therapeutic polynucleotide contains one or more chemical modifications that reduce immunostimulation, enhance serum stability, increase specificity, and/or improve activity, while still retaining silencing activity. Such chemical modifications are described in, for example, Foster et al., RNA. 2012 March; 18(3): 557-568. In some embodiments, the therapeutic polynucleotide contains one or more chemical modifications to prevent degradation, as described in Chen et al., Cell Reports 2012; 2(6)1684-1696.

In a particular embodiment, the therapeutic polynucleotide is selectively delivered to endothelial cells using nanoparticles formulated for selective targeting to endothelial cells, such as a 7C1 nanoparticle. Selective targeting or expression of polynucleotides to an endothelial cell is described in, for example, Dahlman et al., Nat Nanotechnol. 2014 August; 9(8): 648-655.

In some other embodiments, the therapeutic polynucleotide is selectively targeted to a smooth muscle cell. The therapeutic polynucleotide can be selectively delivered to a smooth muscle cell using tissue factor-targeted nanoparticles that can penetrate and bind stretch-activated vascular smooth muscles as described in Lanza et al., Circulation. 2002 Nov. 26; 106(22):2842-7.

### Screening Assays

The treatment strategy described herein using agents that target TGFβ-let-7-FGF signaling in cells (e.g., agents that modulate the activity or level of a TGFβ signaling polypeptide, a let-7 miRNA, or a FGF signaling polypeptide in a cell) can be augmented with a comprehensive new target discovery program that leads to the development of a second generation of therapies targeting the same critical disease-inducing pathway. Accordingly, the present invention further features methods of identifying modulators of a disease, particularly atherosclerosis, comprising identifying candidate agents that interact with and/or alter the level or activity of a TGFβ signaling polypeptide or polynucleotide, a let-7 miRNA, or a FGF signaling polypeptide or polynucleotide in a cell. As described elsewhere herein, the FGF-let-7-TGFβ signaling events drive endothelial-to-mesenchymal transition (EndMT) or smooth muscle cell (SMC) proliferation that contributes to growth of atherosclerotic plaque. Without being bound by theory, it is believed that agents that block or interfere with these molecular events in endothelial cells (e.g., agents that decrease TGFβ signaling) can inhibit development or progression of or reverse atherosclerosis in a subject.

Thus, in some aspects, the invention provides a method of identifying a modulator of atherosclerosis. The method comprises (a) contacting a cell or administering an organism with a candidate agent, and (b) measuring an activity or level of a TGFβ signaling polypeptide or polynucleotide, a let-7 miRNA, or a FGF signaling polypeptide or polynucleotide. An alteration in the level of FGF signaling polynucleotide or polypeptide, a TGFβ polynucleotide or polypeptide, or let-7 miRNA compared with the reference levels, is an indication that the candidate agent is a modulator of atherosclerosis. In particular, a decrease in the activity or level of a TGFβ polynucleotide or polypeptide, or an increase the activity or level of or let-7 miRNA or a FGF signaling polypeptide or polynucleotide in an endothelial cell, would indicate that the candidate agent is an inhibitor of atherosclerosis (e.g., the candidate agent inhibits progression of or reverses atherosclerosis). In some other embodiments, an increase in the activity or level of a TGFβ polynucleotide or polypeptide, or a decrease the activity or level of or let-7 miRNA or a FGF signaling polypeptide or polynucleotide in a smooth muscle cell would indicate that the candidate agent is an inhibitor of atherosclerosis.

Methods of measuring or detecting activity and/or levels of the polypeptide or polynucleotide are known to one skilled in the art. Polynucleotide levels may be measured by standard methods, such as quantitative PCR, Northern Blot, microarray, mass spectrometry, and in situ hybridization. Standard methods may be used to measure polypeptide levels, the methods including without limitation, immunoassay, ELISA, western blotting using an antibody that binds the polypeptide, and radioimmunoassay.

### Kits

The invention provides kits for treating a atherosclerosis in a subject. A kit of the invention provides a therapeutic composition comprising an agent that modulates the activity or level of a TGFβ signaling polypeptide, a let-7 miRNA, or a FGF signaling polypeptide. In particular embodiments, the let-7 miRNA is a human let-7b miRNA chemically modified to increase its stability and reduce an immune response in vivo. In some embodiments, the therapeutic composition further comprises a nanoparticle. In particular embodiments, the nanoparticle is formulated for selective targeting to an endothelial cell. In some embodiments, the nanoparticle is 7C1. In some other embodiments, the nanoparticle is formulated for selective targeting to a smooth muscle cell.

In one embodiment, the kit further includes a diagnostic composition comprising a capture reagent for measuring relative expression level or activity a TGFβ signaling polypeptide, let-7 miRNA, or FGF signaling polypeptide (e.g., a primer or hybridization probe specifically binding to a polynucleotide encoding a TGFβ signaling polypeptide, let-7 miRNA, or FGF signaling polypeptide).

In some embodiments, the kit comprises a sterile container which contains a therapeutic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for using the diagnostic agents and/or administering the therapeutic agents of the invention. In particular embodiments, the instructions include at least one of the following: description of the therapeutic agent; dosage schedule and administration for reducing atherosclerosis symptoms; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

### In General

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Examples

Results of experiments described herein were obtained using the following materials and methods.

### Materials and Methods

### Chemicals

The TGFβR1 kinase inhibitor SB431542 (Sigma 54317) was reconstituted in DMSO (Sigma D2650) and used at a final concentration of 10 μM in cell culture.

### Antibodies

The following antibodies were used for immunoblotting (IB), immunofluorescence (IF) or immunohistochemistry (IHC): Calponin (Sigma C2687; IB, IF), CD31 (Santa Cruz sc-1506; IHC for mouse paraffin samples), CD31 (BD 561814; IHC for mouse fixed OCT samples), CD31 (Dako M0823; IHC for human frozen samples), collagen I (Rockland 600-401-103S; IB), collagen I (Novus Biologicals NB600-408; IHC), Cyclin D1 (Santa Cruz sc-20044; IB), FGFR1 (Epitomics 2144-1; IB), FGFR1 (phospho Y654) (abcam ab59194; IHC), FGFR1 (abeam ab10646; IHC), FRS2α (abeam ab10425; IHC), FRS2α (Santa Cruz sc-8318; IB), GAPDH (glyceraldehyde phosphate dehydrogenase) (Cell Signaling #2118; IB), HSP90 (Sigma 4300541; IB), Ki-67 (Cell Signaling #9027; IHC), myosin (smooth) (Sigma M7786; IB), Notch3 (ab23426, Abcam; IHC), p21 (Cell Signaling #2947; IB), p27 (Cell Signaling #3688; IB), SM22α (abeam ab14106; IB, IF), phospho-Smad2 (Ser465/467) (Cell Signaling #3108; IB), phospho-Smad2 (Ser465/467) (Cell Signaling #3101; IHC for human paraffin samples) (Millipore AB3849; IHC for mouse paraffin samples), phospho-Smad3 (Ser465/467) (R&D AB3226; IB), phospho-Smad3 (Ser465/467) (abeam ab51451; IHC), Smad2/3 (BD 610843; IB), smooth muscle α-actin (Sigma A2547; IB, IHC), smooth muscle α-actin-Cy3 (Sigma C6198; IF), smooth muscle α-actin-APC (allophycocyanin) (R&D IC1420A; IHC), smooth muscle myosin heavy chain 11 (SM-MHC 11) (abeam ab683; IHC), TGFβ (abeam ab66043; IHC), TGFβR1 (Santa Cruz sc-398; IB), TGFβR2 (Santa Cruz sc-400; IB), and P-tubulin (Sigma T7816; IB), F4/80 (abeam ab6640; IHC 1:100), ICAM-1 (BioLegend 116102; IHC for mouse tissue 1:100), smooth muscle α-actin-APC (allophycocyanin) (R&D IC1420A; IHC 1:10), VCAM-1 (abeam ab19569; IHC for mouse tissue 1:1000), and VE-cadherin (Santa Cruz sc-6458; IB 1:100).

### Cell Culture and Reagents

Human 293T T17 cells (human embryonic kidney cells, ATCC CRL-11268) were maintained in Dulbecco's modified Eagle's medium (Gibco 11965-092) with 10% fetal bovine serum (Life Technologies 16000-044) and penicillin-streptomycin (15140-122, Gibco), and were grown at 37° C. 5% CO2. Human aortic smooth muscle cells (#C-007-5C), media (#M231-500), and supplements (SMGS: S-007-25; SMDS: S-008-5) were purchased from Life Technologies. The cells were grown at 37° C., 5% CO₂ in Medium 231 supplemented with smooth muscle growth supplement (SMGS containing 4.9% FBS, 2 ng/ml FGF2, 0.5 ng/ml EGF, 5 ng/ml heparin, 2 μg/ml IGF-1, and 0.2 μg/ml BSA). For SMC differentiation, HASMC were incubated with Medium 231 containing smooth muscle differentiation supplement (SMDS containing 1% FBS and 30 μg/ml heparin) for different time points. Primary human aortic smooth muscle cells between passages 6 and 10 were used in all experiments. Mouse bEnd.3 cells (ATCC CRL-2299) were maintained in Dulbecco's modified Eagle's medium (ATCC 30-2002) with 10% fetal bovine serum (Life Technologies 16000-044) and penicillin-streptomycin (Gibco 15140-122), and were grown at 37° C., 5% CO₂. Primary mouse endothelial cells were isolated from the hearts and lungs using rat anti-mouse CD31 antibody (BD #553370) and Dynabeads (Invitrogen 110.35). Briefly, minced hearts and lungs were digested with Type I collagenase (2 mg/ml; Sigma C0130) at 37° C. for 45 min with agitation. The cells were then filtered through a 70 μm disposable cell strainer (BD Falcon 352350), and centrifuged at 1300 rpm for 10 min at 4° C., and then resuspended in 2 ml of EC medium [DMEM (LONZA 12-709F), 20% FBS (Sigma 26140-079), 10 units/ml Penicillin/10 μg/ml Strep (Gibco 15140-122), 1× non-essential amino acid (Gibco 11140-050), 2 mM L-glutamine (Gibco 25030-081), 1.2 μg/ml Amphotericin B (Fisher Scientific BP2645-50), 60 μg/ml Gentamycin sulfate (Gibco 15750-060)]. The cells were then incubated with anti-mouse CD31 Dynabeads on a rotator at room temperature for 15 min. After several washes, the cells were plated on gelatin-coated 10 cm dishes or they were centrifuged at 1300 rpm for 10 min at 4° C. and storage in −80° C. freezer. Feed the cells with EC medium containing 100 mg/ml heparin (Sigma H-3933), 100 mg/ml ECGS (Alfa Aesar J64516). Primary mouse endothelial cells between passages 3 and 4 were used in all experiments.

### Growth Factors and Chemicals.

Recombinant human BMP9 (553104, BioLegend), recombinant mouse IFN-γ (315-05, Peprotech), recombinant human IL-1β (200-01B, Peprotech), recombinant human IL-6 (AF-200-06, Peprotech), recombinant human TGFβ 1 (580702, BioLegend), and recombinant human TNF-α (300-01A, Peprotech) were reconstituted in 0.1% BSA/PBS.

### Generation of Lentiviruses

Mouse Tgfbr1 and Tgfbr2 shRNA lentiviral constructs were purchased from Sigma. Human FGFR1, human Smad2 and human TGFβR2 shRNA lentiviral constructs were purchased from Sigma and human FRS2α shRNA lentiviral construct was purchased from Open Biosystems. For the production of shRNA lentivirus, 3.7 μg of Δ8.2, 0.2 μg of VSVG, and 2.1 μg of pLKO.1 carrying the control, FGFR1, FRS2α, Smad2, or TGFβR2 shRNA were co-transfected into 293T cells using X-tremeGENE 9 DNA Transfection Reagent (Roche 06365787001). Forty-eight hours later the medium was harvested, cleared by 0.45 μm filter (PALL Life Sciences PN4184), mixed with polybrene (5 μg/ml) (Sigma H9268), and applied to cells. After 6 hr. incubation, the virus-containing medium was replaced by the fresh medium. For production of let-7 miRNA lentivirus, 10 μg of pMIRNA1 carrying the let-7b (PMIRHlet7bPA-1) miRNA expression cassette (System Biosciences), 5 g of pMDLg/PRRE, 2.5 g of RSV-REV, and 3 g of pMD.2G were co-transfected into 293T cells using X-tremeGENE 9 DNA transfection reagent (Roche 06365787001). Forty-eight hr. later, the medium was harvested, cleared by 0.45 m filter (PALL Life Sciences PN4184), mixed with 5 g/ml polybrene (Sigma H9268), and applied to cells. After 6 hr incubation, the virus-containing medium was replaced by fresh medium.

RNA Isolation and qRT-PCR

Cells were suspended in TRIzol Reagent (Invitrogen #15596018), and total RNA (#74134, QIAGEN) and miRNA-enriched fraction (#74204, QIAGEN) were isolated according to the manufacturer's instructions. Reverse transcriptions were performed by using iScript cDNA synthesis kit (170-8891, Bio-Rad) for mRNA or RT2 miRNA First Strand Kit (331401, QIAGEN) for miRNA. qRT-PCR was performed using Bio-Rad CFX94 (Bio-Rad) by mixing equal amount of cDNAs, iQ SYBR Green Supermix (Bio-Rad 170-8882) and gene specific primers (QIAGEN), 3-actin [PPM02945B], Tgfbr1 [PPM03072C], Tgfbr2 [PPM03599B], mmu-let-7b [MPM00484A], and SNORD66 [MPM01662A]. All reactions were done in a 20-25 μl reaction volume in duplicate. Individual mRNA or miRNA expression was normalized in relation to expression of endogenous 3-actin or small nuclear SNORD47/SNORD66, respectively. PCR amplification consisted of 10 min of an initial denaturation step at 95° C., followed by 46 cycles of PCR at 95° C. for 15 s, 60° C. for 30 s (for mRNA cDNA) and 10 min of an initial denaturation step at 95° C., followed by 46 cycles of PCR at 95° C. for 15 s, 55° C. for 30 s, 70° C. for 30 s (for miRNA cDNA).

### Western Blot Analysis

Cells were lysed with HNTG lysis buffer (20 mM HEPES, pH 7.4/150 mM NaCl/10% glycerol/1% Triton-X 100/1.5 mM MgCl2/1.0 mM EGTA) containing complete mini EDTA-free protease inhibitors (Roche #11836170001) and phosphatase inhibitors (Roche #04906837001). 20 g of total protein from each sample was resolved on Criterion TGX Precast Gels (Bio-Rad #567-1084) with Tris/Glycin/SDS Running Buffer (Bio-Rad #161-0772), transferred to nitrocellulose membranes (Bio-Rad #162-0094) and then probed with various antibodies. Chemiluminescence measurements were performed using SuperSignal West Pico Chemiluminescent Substrate (Thermo Fisher Scientific Prod #34080).

### Immunofluorescence Staining

Cultured primary human aortic smooth muscle cells were grown on 10 μg/ml fibronectin (Sigma F2006) coated glass-bottomed dishes (MatTek CORPORATION P35G-1.5-20-C). Cells were first fixed with 2% paraformaldehyde (Polysciences, Inc, 18814) in PBS for 20 minutes at 37° C. then permeabilized with 0.1% triton X-100 in PBS containing 2% PFA at room temperature for 5 minutes, and blocked with 3% bovine serum albumin (Jackson ImmunoResearch Laboratories, Inc. 001-000-162) at room temperature for 60 minutes. Cells were washed with PBS and incubated with SM α-actin-Cy3 (1:1000 in 1% BSA), SM22α (1:1000 in 1% BSA), and SM-calponin (1:500 in 1% BSA) antibodies at 4° C. overnight, washed three times with PBS and incubated with diluted Alexa Fluor-conjugated secondary antibody (1:500) (life technologies) for 1 hour at room temperature. The dishes were then washed three times with PBS and mounted using Prolong Gold antifade reagent with DAPI (life technologies P36935).

### Cell Contraction Assay

Cell contraction assay was evaluated using a Cell Contraction Assay Kit according to the manufacturer's instructions (CELL BIOLABS-CBA-201). Briefly, HASMCs were harvested and suspended at 5×10⁵ cells/ml, and the collagen lattice was prepared by mixing two parts of cell suspension and eight parts of cold collagen gel solution. Subsequently, 500 μl of the cell-collagen mixture was cast into each well of a 24-well plate and allowed to polymerize at 37° C. for 1 hr. After collagen polymerization cells were incubated in SMC growth medium (Medium 231 plus SMGS) for 24 hr. during which stress developed. Upon release of the collagen lattice from the culture dish, the embedded cells become free to contract the deformable lattice thus reducing its surface area. This was quantified twenty-four hr. after detachment of the gel from the dish using ImageJ and expressed as the percentage of the area of the entire well.

xCELLigence® Real-Time Cell Analysis (RTCA)

Cell proliferation experiments were carried out using the xCELLigence® RTCA DP instrument (Roche Diagnostics GmbH) in a humidified incubator at 37° C. and 5% CO₂. Cell proliferation experiments were performed using modified 16-well plates (E-plate, Roche Diagnostics GmbH). Initially, 100 μL of cell-free growth medium was added to the wells. After leaving the devices at room temperature for 30 min, the background impedance for each well was measured. 100 μL of the cell suspension was then seeded into the wells (1000 cells/well). Plates were locked in the RTCA DP device in the incubator and the impedance value of each well was automatically monitored by the xCELLigence system and expressed as a Cell Index value (CI). CI was monitored every 15 min for 600 times. Two replicates of each cell concentration were used in each test. All data have been recorded by the supplied RTCA software (vs. 1.2.1).

### Cell Cycle Analysis

Cell cycle analysis was performed using propidium iodide (PI) staining and flow cytometry. Cells were trypsinized, washed twice in PBS and fixed in 70% ethanol at −20° C. overnight. After washing twice in PBS, the cells were treated with 100 g/ml RNase A (Sigma R5125) at 37° C. for 30 min and stained in 50 g/ml PI solution (Sigma P4170). Then the cells were transferred to flow cytometry tubes with filters (BD #352235) for cell cycle analysis. 10,000 events were collected for each sample. The data were collected and analyzed with FlowJo software (Tree Star).

### Patient Population

Human coronary arteries were obtained from the explanted hearts of transplant recipients or cadaver organ donors. Research protocols were approved by the Institutional Review Boards of Yale University and the New England Organ Bank. A waiver for consent was approved for surgical patients and written informed consent was obtained from a member of the family for deceased organ donors. Table 1 summarizes clinical characteristics of this patient group.

### Specimen Collection

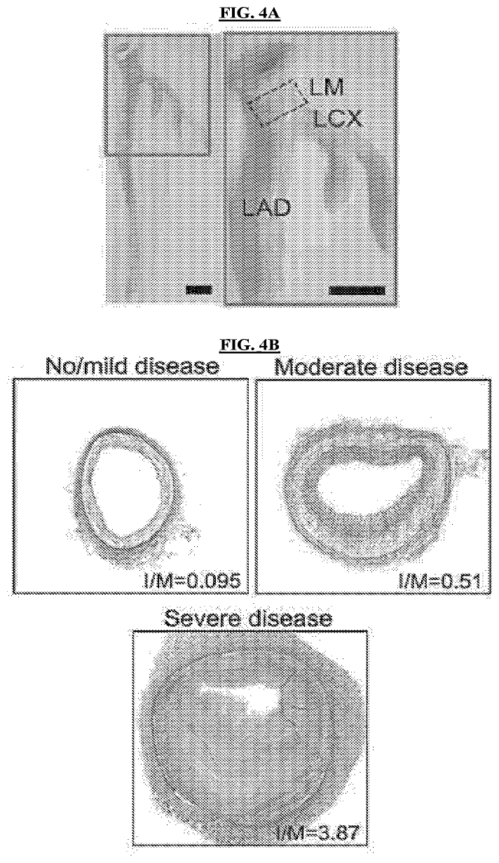

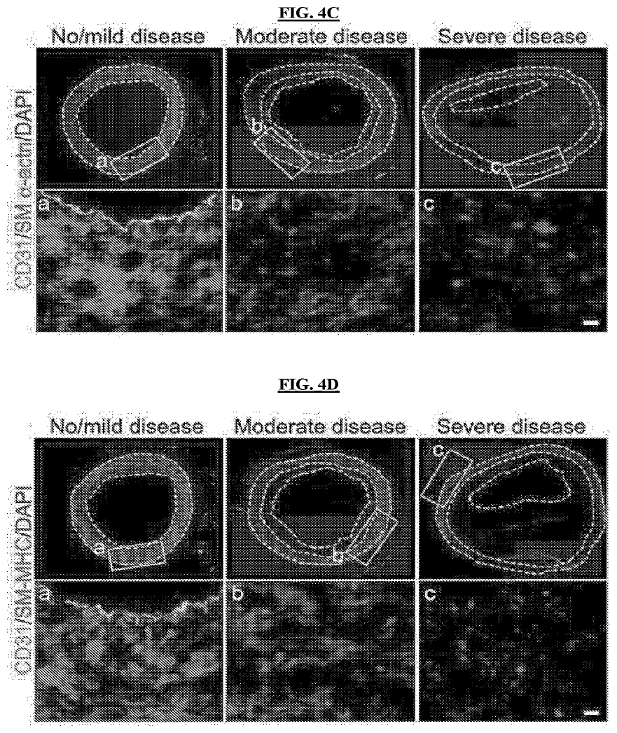

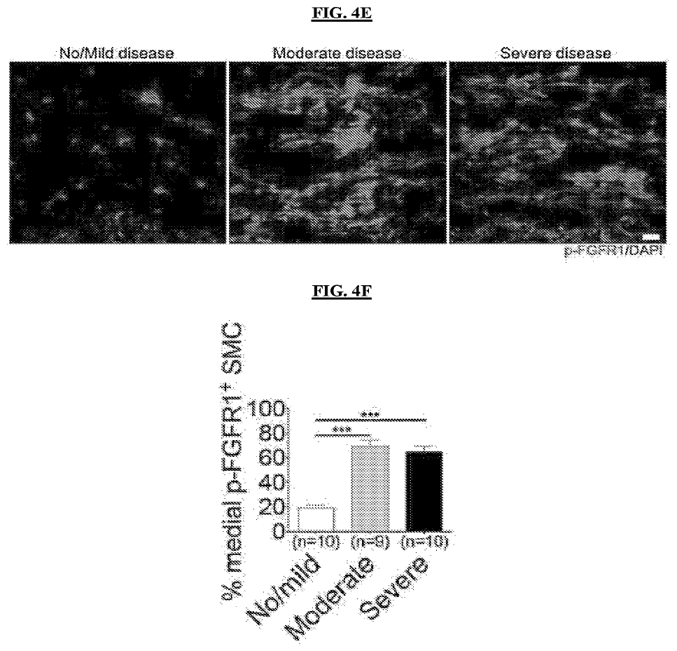

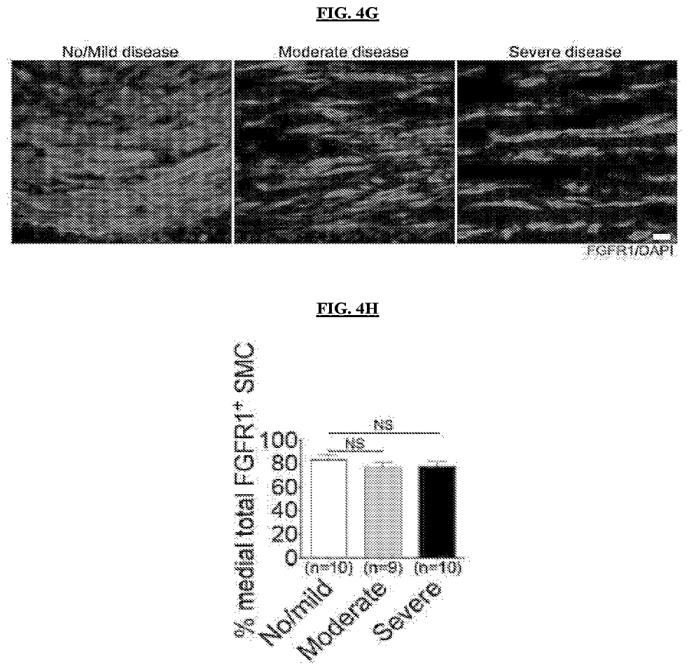

Investigators were on call with the surgical team and collected the heart at the time of explant. To minimize ex vivo artifacts, a ˜5-20 mm segment of the left main coronary artery was removed within the operating room (FIG. 4A) and immediately processed as frozen sections in Optimal Cutting Temperature medium and, when of sufficient length, an additional segment was also fixed in formalin for later embedding, sectioning, and staining.

### Generation of Mice

Cdh5-CreER^{T2} mice were obtained from R. Adams (Max Planck Institute), Tgfbr2^{fl/fl} mice were obtained from Harold L. Moses (Vanderbilt University), and Tgfbr1^{fl/fl} mice were obtained from Martin M. Matzuk (Baylor College). To generate Cdh5-CreER^{T2}; Tgfbr1^{fl/fl}-Tgfbr2^{fl/fl} mice, Cdh5-CreER^{T2}.; Tgfbr2^{fl/fl} mice were mated with Tgfbr1^{fl/fl} mice. To generate Cdh5-CreER^{T2}; Tgfbr1^{fl/fl}-Tgfbr2^{fl/fl}-mT/mG mice, Cdh5-CreER^{T2}; Tgfbr1^{fl/fl}-Tgfbr2^{fl/fl}-mT/mG mice were mated with mT/mG mice (B6.129(Cg)-Gt(ROSA)26Sor^{tm4(ACTB-tdTomato,-EGFP)Luo/J}, Stock No: 007676, The Jackson Laboratory). To generate Cdh5-CreER^{T2}; Tgfbr1^{fl/fl}-Tgfbr2^{fl/fl}-Apoe^{-/-}mT/mG mice, Cdh5-CreER^{T2}; Tgbr1^{fl/fl}-Tgfbr2^{fl/fl}-mT/mG mice were mated with Apoe^{-/-} mice (B6.129P2-Apoetm1Unc/J, Stock No: 002052). C57BL/6J (Stock No: 000664) and Apoe^{-/-} mice (B6.129P2-Apoetm1Unc/J, Stock No: 002052) were purchased from The Jackson Laboratory. This strain had been back-crossed more than ten times to C57BL/6 background. All animal procedures were performed under protocols approved by Yale University Institutional Animal Care and Use Committee.

Frs2α^{flox/flox} mice were previously described (Lin et al, 2007, Genesis 45: 554-559). Frs2α^{flox/flox} mice were bred with mice expressing Cre recombinase under the SM22α promoter. SM22α^{flox/flox} offspring were crossed to C57BL6 Apoe^{-/-} mice (JAX SN:002052). Genotyping was performed by mouse tail DNA PCR analysis. Mouse tail DNA was isolated using the DNeasy Blood & Tissue kit (QIAGEN #69506). PCR genotyping analysis was done using the following primers: Frs2^{flox/flox} (5′-GAGTGTGCTGTGATTGGAAGGCAG-3′(SEQ ID NO: 1) and 5′-GGCACGAGTGTCTGCAGACACATG-3′ (SEQ ID NO: 2)), SM22α-Cre (5′-GCG GTC TGG CAG TAA AAA CTA TC-3′ (SEQ ID NO: 3), 5′-GTG AAA CAG CAT TGC TGT CAC TT-3′ (SEQ ID NO: 4), 5′-CTA GGC CAC AGA ATT GAA AGA TCT-3′ (SEQ ID NO: 5), and 5′-GTA GGT GGA AAT TCT AGC ATC ATC C-3′ (SEQ ID NO: 6)), Apoe (5′-GCCTAGCCGAGGGAGAGCCG-3′ (SEQ ID NO: 7), 5′-GTGACTTGGGAGCTCTGCAGC-3′ (SEQ ID NO: 8), and 5′-GCCGCCCCGACTGCATCT-3′ (SEQ ID NO: 9)), Cdh5-CreER^{T2} (5′-GCC TGC ATT ACC GGT CGA TGC AAC GA-3′ (SEQ ID NO: 10), and 5′-GTG GCA GAT GGC GCG GCA ACA CCA TT-3′ (SEQ ID NO: 11)), Tgfbr1^{fl/fl} (5′-ACT CAC ATG TTG GCT CTC ACT GTC-3′ (SEQ ID NO: 12), and 5′-AGT CAT AGA GCA TGT GTT AGA GTC-3′ (SEQ ID NO: 13), Tgfbr2^{fl/fl} (5′-TAA ACA AGG TCC GGA GCC CA-3′ (SEQ ID NO: 14), and 5′-ACT TCT GCA AGA GGT CCC CT-3′ (SEQ ID NO: 15)), and mT/mG (5′-CTC TGC TGC CTC CTG GCT TCT-3′ (SEQ ID NO: 16), 5′-CGA GGC GGA TCA CAA GCA ATA-3′ (SEQ ID NO: 17), and 5′-TCA ATG GGC GGG GGT CGT T-3′ (SEQ ID NO: 18)).

All animal procedures were performed under protocols approved by Yale University Institutional Animal Care and Use Committee.

### Echocardiographic Studies

Experiments were performed at the Yale Translational Research Imaging Center Core Facility. Cardiac function was analyzed by echocardiography using a Vevo 770® console (VisualSonics). Mice body temperature was maintained with a heading pad. Mice were anesthetized with 2% isoflurane, maintained under anesthesia with 1% isoflurane, and examined. The mouse was placed chest up on an examination board interfaced with the Vevo 770® console. Warmed Aquasonic gel was applied over the thorax and a 30-MHz probe was positioned over the chest in a patasternal position. Long and short axis B-mode and M-mode images were recorded. All measurements were obtained from three to six consecutive cardiac cycles, and the averaged values were used for analysis. Upon completion of the procedure, the gel was wiped off and the animal was returned to its cage housed in a warm chamber.

### Serum Lipid Analysis

Serum was obtained through centrifugation of the blood for 2 min at 10,000 rpm at 4° C. and stored at −80° C. until each assay was performed. Total cholesterol and triglycerides were performed in the Yale Mouse Metabolic Phenotyping Center.

### Histology and Morphometric Analysis

The animals were euthanized and perfusion-fixed with 4% paraformaldehyde (Polysciences, Inc. Cat #18814) via the left ventricle for 5 min. For human vessel studies, sections of left main coronary arteries were stained with Elastic Van Gieson (EVG). Digital EVG-stained photographs of one section from each block were projected at final magnifications of X100. ImageJ software (NIH) was used for morphometric analyses. As described in FIG. 4B, measurements were made of the intima and media thickness. The ratio of intima (I) to media (M) thickness was used to grade the severity of atherosclerosis. The results for these parameters from each specimen were average of four different areas to obtain mean values. Left main coronary arteries of I/M ratio less than 0.2 were considered as no disease or mild disease; those of I/M ratio between 0.2-1 were considered as moderate disease; those of I/M ratio greater than 1 or have calcification as severe disease.

### Histological Analysis of Atherosclerotic Lesions

Apoe^{-/-} and Frs2^{SMCKO}/Apoe^{-/-} male mice were fed a Western diet (40% kcal % Fat, 1.25% Cholesterol, 0% Cholic Acid) for 8 or 16 weeks (Research Diets, product #D12108) starting at the age of 8 weeks. After 8 or 16 weeks of being fed a high-fat diet, mice were anesthetized and euthanized. Mouse heart were perfused with 10 ml of Dulbecco's Phosphate Buffered Saline (DPBS) (Life Technologies Cat #14190-144) and 10 ml of 4% paraformaldehyde (Polysciences, Inc. Cat #18814) via the left ventricle. The lesions located in the aorta, aortic roots and abdominal aorta were analyzed using Oil Red 0 staining. To measure lesions in the aorta, the whole aorta, including the ascending arch, thoracic and abdominal segments, was dissected, gently cleaned of adventitial tissue and stained with Oil Red 0 (Sigma 00625) as previously described (Huang et al, 2013, Arterioscler Thromb Vase Biol 33: 795-804). The surface lesion area was quantified with ImageJ software (NIH). To measure lesions in the aortic root, the heart and proximal aorta were excised, and the apex and lower half of the ventricles were removed.

### Immunohistochemical Staining

Blocks were sectioned at 5 μm intervals using a Microm cryostat (for frozen blocks) or a Paraffin Microtome (for paraffin blocks). For frozen tissue sections, slides were fixed in acetone for 10 min at −20° C. For paraffin sections, slides were dewaxed in xylene, boiled for 20 min in citrate buffer (10 mM, pH 6.0) for antigen retrieval, and rehydrated. After washing three times with phosphate-buffered saline, tissue sections were incubated with primary antibodies diluted in blocking solution (10% BSA and horse serum in PBS) overnight at 4° C. in a humidified chamber. For p-Smad2, p-Smad3 staining, slides were denatured with 1.5 M HCl for 20 min prior to antibody labeling.

Sections were washed three times with tris-buffered saline, incubated with appropriate Alexa Fluor 488-, Alexa Fluor 594-, or Alexa Fluor 647-conjugated secondary antibodies diluted 1:1000 in blocking solution for 1 hr at room temperature, washed again three times, and mounted on slides with ProLong Gold mounting reagent with DAPI (Life Technologies P36935). All immunofluorescence micrographs were acquired using a Zeiss microscope. Images were captured using Velocity software and quantifications performed using ImageJ software (NIH).

### Statistical Analysis

Graphs and statistical analysis were prepared using GraphPad Prism software. Data are expressed as mean±SD. The level of statistical significance was determined by one-way ANOVA with Newman-Keuls test for multiple comparisons or 2-tailed Student's t test using the GraphPad Prism software. A P value less than 0.05 was considered significant (*P<0.05, **P<0.01, ***P<0.001). All results were confirmed by at least 3 independent experiments. Error bars represent mean±SD.

### Study Approval

All experiments involving animals were reviewed and approved by the Animal Welfare Committee of Yale University. The ethics committee of Yale University approved the procedures related to human subjects. All patients who participated in the study provided written informed consent.

### Synthesis of Let-7b Mimics

Chemically-modified miRNA mimics were synthesized at Alnylam Pharmaceuticals (Cambridge, MA). The sequences for the mature strands of let-7b after processing by DICER, mmu-let-7b-5p (mmu-let-7b, MIMAT0000522), and mmu-let-7b-3p (mmus-let-7b*, MIMAT0004621), were obtained from the miRbase (http://www.miRbase.org). 2-O-methyl-nucleotide modifications (indicated in lower case) were introduced to both strands to decrease the likelihood of triggering an innate immune response. Double stranded miRNA mimics were obtained after annealing equimolar amounts of the chemically-modified 5p and 3p strands: mi-let-7b_{L}, for lightly modified (5p 5′-UGAGGuAGuAGGUUGUGUGGUU-3′ (SEQ ID NO: 19), 3p 5′-CuAuAcAACCuACUGCCUUCCC-3′ (SEQ ID NO: 20); and mi-let-7b_{H}, for heavily modified, (5p 5′-UGAGGuAGuAGGUUGUGUGGUU-3′ (SEQ ID NO: 19), 3p 5′-cuAuAcAAccuAcuGccuuccc-3′ (SEQ ID NO: 20). LNPs formulated with siRNA targeting luciferase, siLuc, were used as control. The siLuc, which was also incorporated 2-O-methyl-nucleotide modifications, is commonly used as a control for in vivo siRNA and miRNA studies (Dahlman et al., 2014, Nat Nanotechnol 9, 648-655; Sager et al., 2016, Science translational medicine 8, 342ra380) let-7 mimics have been validated previously in vivo (Chen et al., 2012, Cell reports 2: 1684-1696).

Synthesis of siTgfbr1 and siTgfbr2.

Chemically-modified siRNA against mouse Tgfbr1 and Tgfbr2 were synthesized at Alnylam Pharmaceuticals (Cambridge, MA). The siRNA sequence for Tgfbr1 sense strand (UGUCAAGGAGAUGCUUCAAuAdTsdT) (SEQ ID NO: 35) and antisense (UAUUGAAGCAUCUCCUUGACAUAdTsdT) (SEQ ID NO: 36); for Tgfbr2 is sense (GGCUCGCUGAACACUACCAAAdTsdT) (SEQ ID NO: 37) antisense (UUUGGUAGUGUUCAGCGAGCCAUdTsdT) (SEQ ID NO: 38). miRNA formulation in lipid nanoparticles (LNPs). siRNA targeting Tgfbr1, Tgfbr2, and siLuc were encapsulated in LNPs formulated with the lipid 7C1, using the same protocol and composition as previously described (Dahlman, 2014, Nat Nanotechnol 9, 648-655). More specifically, 7C1 was synthesized and purified as previously described (Dahlman, 2014, Nat Nanotechnol 9, 648-655). It was then combined with C₁₄PEG₂₀₀₀ in a glass syringe (Hamilton Company), and diluted with 100% ethanol. let-7 mimics or siLuc were diluted in 10 mM citrate buffer, and loaded into a separate syringe. The two syringes were connected to a microfluidic mixing device (Chen et al, 2012 J Am Chem Soc. 2012 134(16):6948-51), before the 7C1 and RNA solutions were mixed together at a flow rate of 600 and 1800 μL/min, respectively. The resulting nanoparticles were dialyzed into 1×PBS, before being sterile filtered using a 0.22 μm filter.

### Animal Treatment.

Cre-Lox recombination was induced by tamoxifen (Sigma T5648) at 1 mg/day i.p. for 5 days versus vehicle (corn oil, Sigma C8267) alone. For PBS, siluciferase, let-7 mimics and siTgfbr1/Tgfbr2 delivery in mouse atherosclerosis model, 8 to 10 week old mice were placed on a Western diet (40% kcal % Fat, 1.25% Cholesterol, 0% Cholic Acid) for 16 weeks (Research Diets, product #D12108) and injected intravenously every 10 days during this period of the following: sterile PBS (100 μl/mouse), luciferase-control (2 mg/kg), siTgfbr1/Tgfbr2 (2 mg/kg) or let-7b mimics. For LPS administration, mice were given Escherichia coli LPS (Sigma L2630) prepared in 0.1 ml of sterile saline and administered i.p. by single injection at a dose of 100 μg/kg. Animals were studied 3 h after the injections. For Rapamycin (Millipore 553210) treatment, 8 to 10 week old Apoe^{-/-} mice were placed on a Western diet (40% kcal % Fat, 1.25% Cholesterol, 0% Cholic Acid) for 16 weeks (Research Diets, product #D12108) and injected at 2 mg/kg/d i.p. every day (q.d.). 4% DMSO injected mice was used as controls. Control groups received 0.1 ml of saline i.p.

The results of experiments are now described.

### Example 1: FRS2α Regulates TGFβ Activity and SMC Differentiation

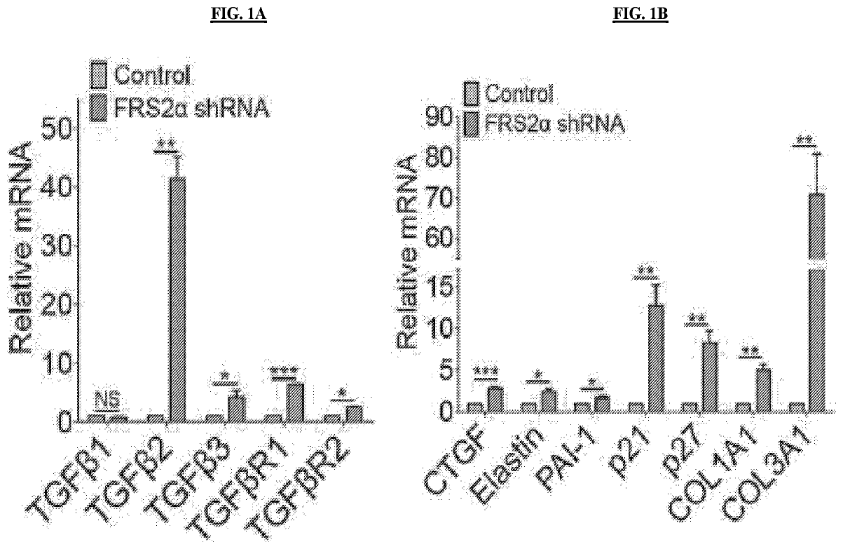

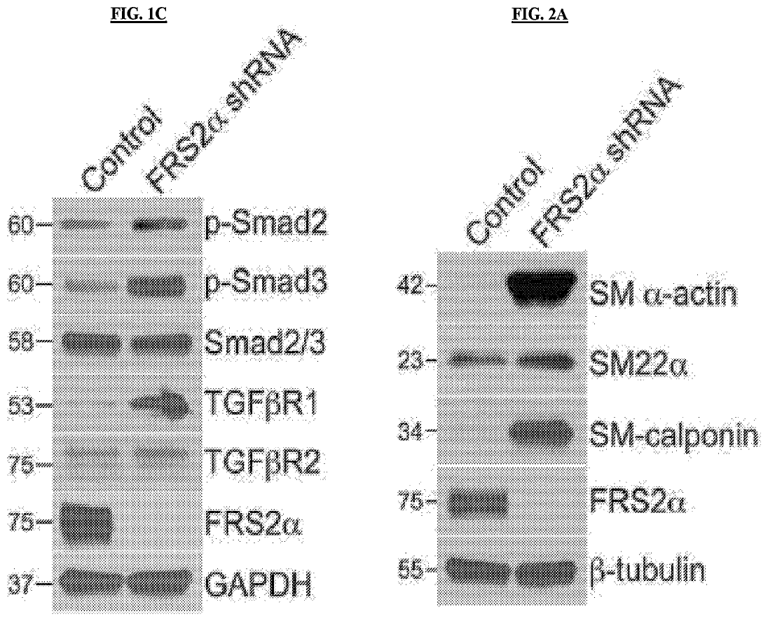

Inhibition of FGF signaling in SMCs using FRS2α knockdown and its effect on the expression of TGFβ pathway signaling molecules was examined. In cultured human aortic smooth muscle cells (HASMCs), knockdown of FRS2α led to a significant increase in expression of TGFβ2, TGFβ3, TGFβR1, and TGFβR2 (FIG. 1A). TGFβ 1 was unchanged. In addition, there was an increase in the expression of a number of TGFβ-dependent genes including connective tissue growth factor (CTGF), elastin, plasminogen activator inhibitor-1 (PAI-1), p21, p27, and collagen (FIG. 1B) suggesting activation of TGFβ signaling. This was confirmed by Western blotting that demonstrated increase phosphorylation of Smad2 and Smad3 following FRS2α knockdown (FIG. 1C).

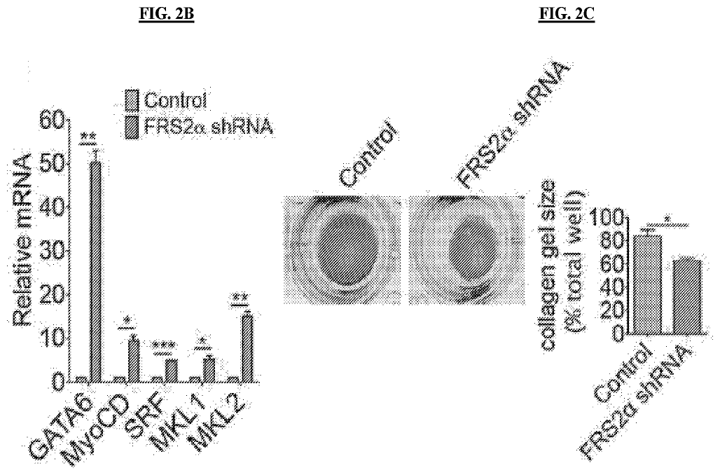

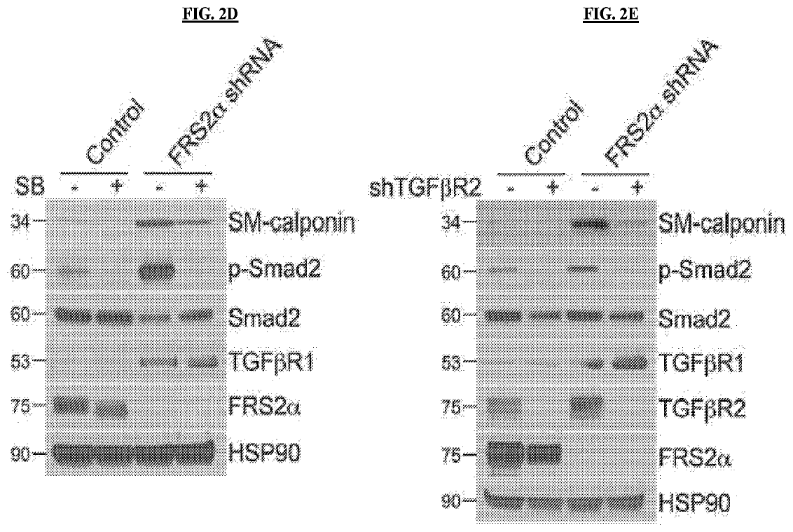

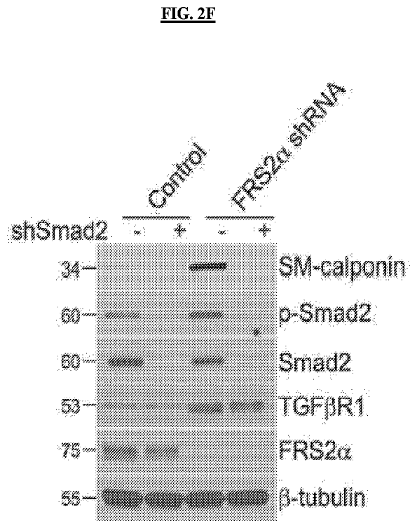

Cultured SMCs in serum-supplemented medium lose differentiation marker expression and acquire a synthetic (proliferative) phenotype. Since activation of TGFβ signaling has been linked with the induction of SMC differentiation, differentiation marker expression was next examined in cultured HASMC following FRS2α knockdown. There was a pronounced increase in expression of SM α-actin, SM22α and SM-calponin (FIG. 2A) as well as various transcription factors (GATA6, MyoCD, SRF) and transcription co-activators (MKL1, MKL2) responsible for the induction of contractile phenotype (FIG. 2B). The contractile machinery was functional as observed by increased contraction of collagen gels following FRS2α knockdown (FIG. 2C).

To assess the effect of FGF signaling shutdown on SMC proliferation, real time cell analysis was used to track HASMC growth in the presence and absence of FRS2α knockdown. The absence of FRS2α expression resulted in nearly complete inhibition of serum-induced HASMC proliferation (FIG. 9A). Western blot analysis demonstrated a decrease in the proliferative marker Cyclin D1 whereas expression of cell cycle inhibitor proteins p21 and p27 was upregulated (FIG. 9B). In agreement with these findings, FACS analysis showed a G1/S arrest following FRS2α knockdown (FIG. 9C).

To test if TGFβ activity is required for FRS2α knockdown-induced SMC differentiation, HASMCs were exposed to FRS2α or control shRNA lentiviruses in the presence or absence of the TGFβR1 kinase inhibitor, SB431542. The inhibitor treatment effectively attenuated FRS2α knockdown-induced increase in p-Smad2 and SM-calponin levels (FIG. 2D) demonstrating that TGFβ activity is essential for FRS2α knockdown-induced contractile smooth muscle gene expression. This was further confirmed by shRNA-mediated knockdown of TGFβR2 or Smad2 with both knockdowns preventing increase in SM-calponin expression (FIGS. 2E-2F).

### Example 2: FGFR1 and Let-7 Mediate FGF-Driven Suppression of TGFβ Signaling in SMCs

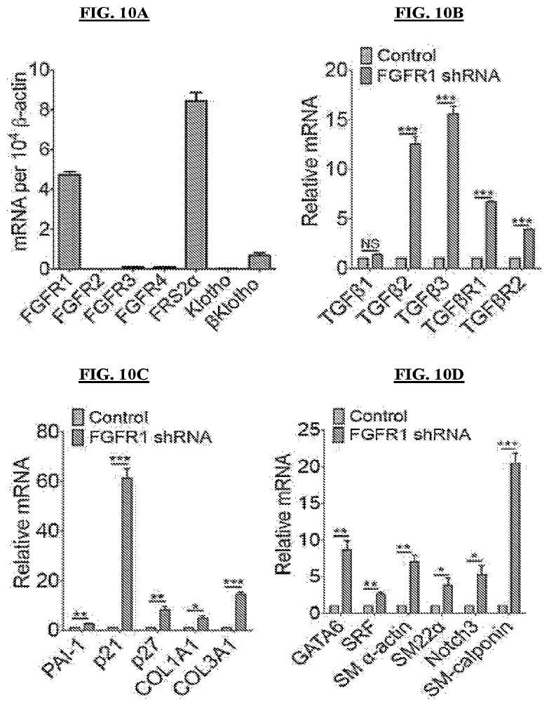

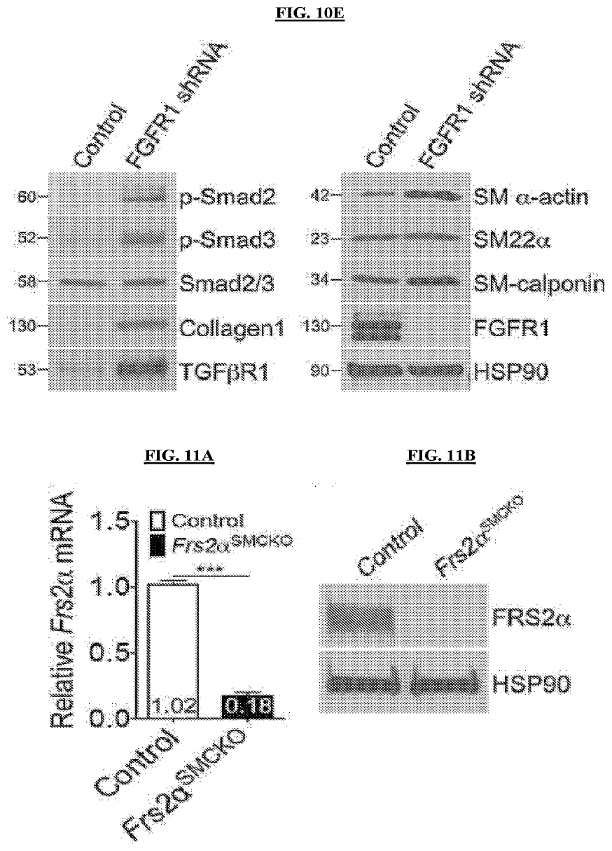

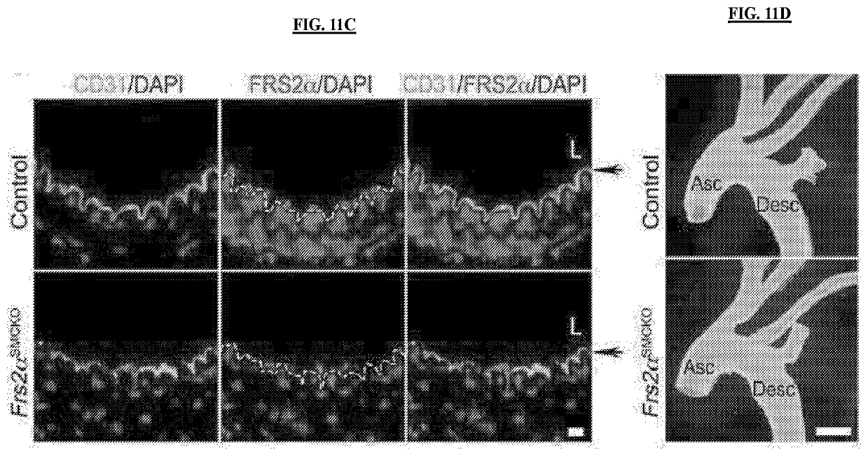

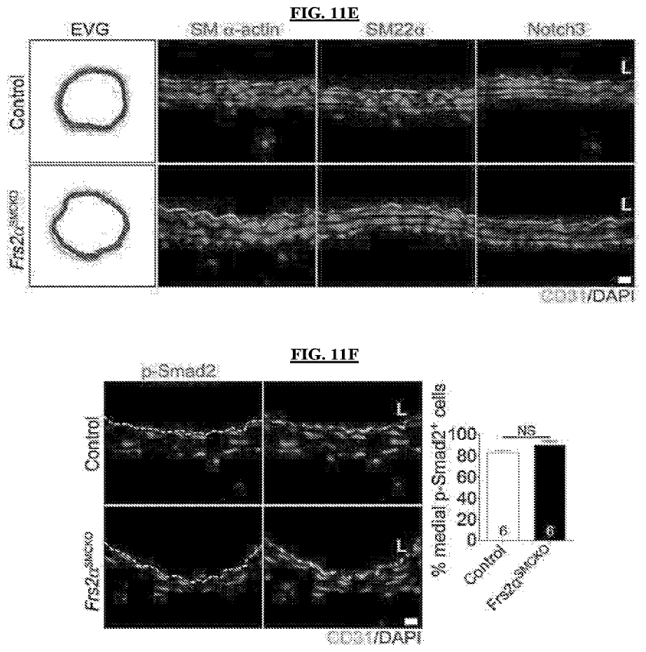

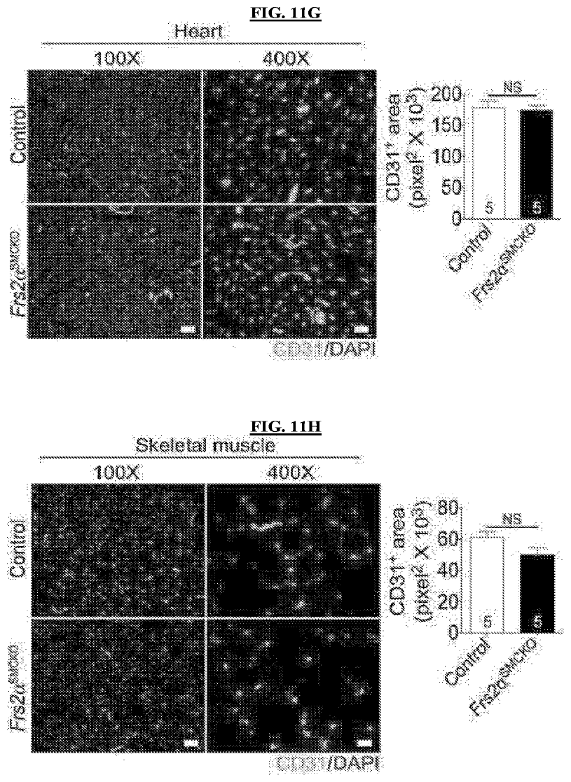

FRS2α is involved in signaling of all four FGF receptors. The following experiments were conducted to establish the principal FGFR responsible for suppression of TGFβ signaling in SMC. qPCR analysis demonstrated that FGFR1 was the main FGFR expressed in cultured HASMCs (FIG. 10A). In agreement with that finding, shRNA-mediated FGFR1 knockdown markedly increased TGFβ2, TGFβ3, TGFβR1 and TGFβR2 expression (FIG. 10B) in a manner similar to that of the FRS2α knockdown. This also led to activation of TGFβ signaling as demonstrated by increased expression of a number of TGFβ-dependent genes and transcription factors (FIGS. 10C-10D). Western blotting confirmed activation of TGFβ signaling as demonstrated by increased Smad2 and Smad3 phosphorylation and increased contractile SMC gene expression (FIG. 10E).

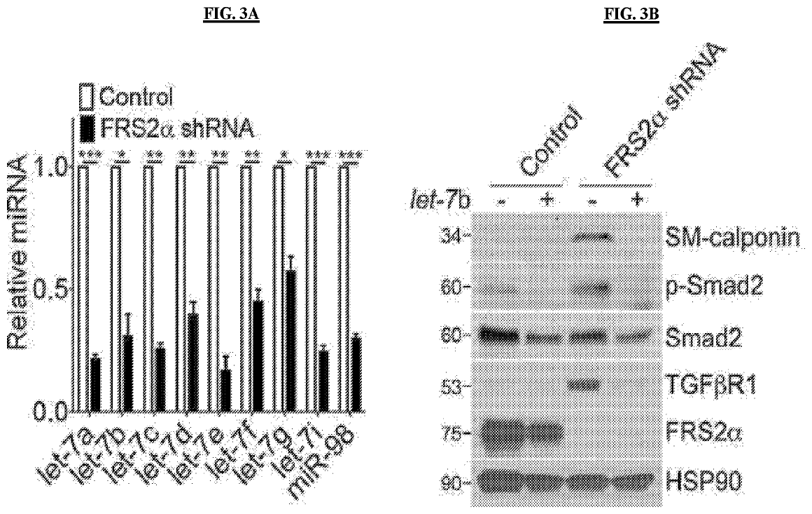

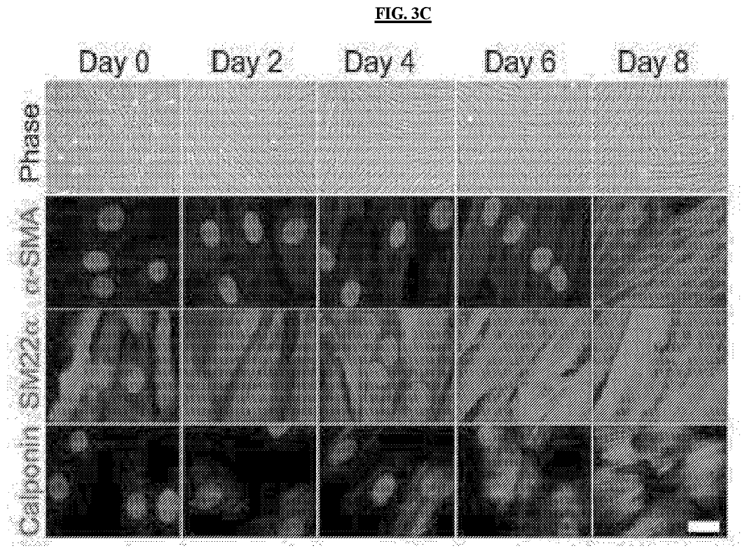

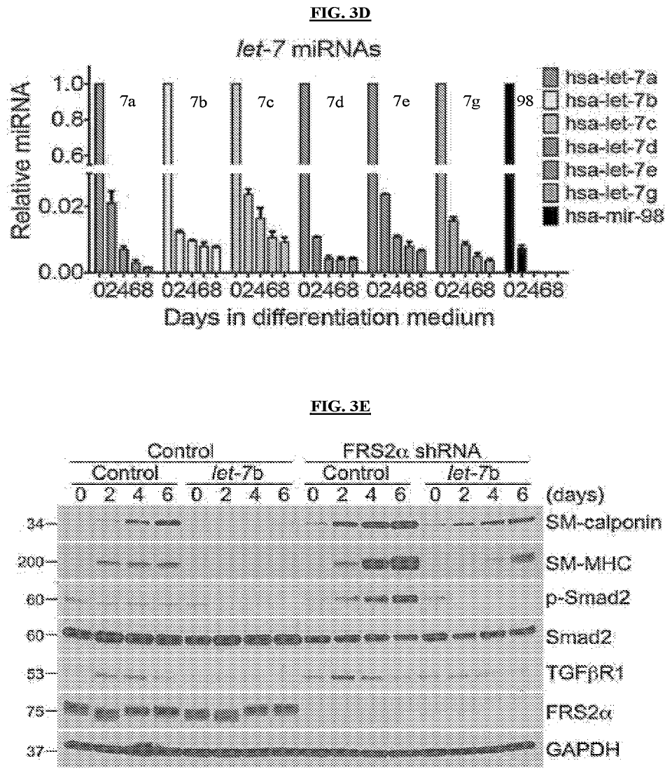

It was previously showed that suppression of FGF signaling in endothelial cells decreases expression of let-7 miRNA family members (Chen et al, 2012, Cell reports 2: 1684-1696; Chen et al, 2014, Science signaling 7: ra90). To assess if the same mechanism is operational in SMCs, let-7 levels were examined after shRNA-mediated FRS2α knockdown in HASMCs. As in endothelial cells, this led to a substantial decrease in let-7 miRNAs expression in FRS2α knockdown HASMCs (FIG. 3A). Transduction of let-7b into HASMCs following FRS2α knockdown prevented activation of TGFβ signaling as demonstrated by decreased TGFβR1, p-Smad2 and SM-calponin levels (FIG. 3B).

Growth arrest of cultured SMCs has been shown to induce their conversion from proliferative to contractile phenotype (Clowes et al, 1988, J Cell Biol 107: 1939-1945). Indeed, shifting HASMC cultured in 4.9% FBS to 1% FBS medium led to a gradual increase in expression of contractile SMC proteins (FIG. 3C). Analysis of let-7 family members' expression during HASMC differentiation demonstrated a profound decrease that preceded changes in contractile proteins expression suggesting let-7-dependent control of this process (FIG. 3D).

To test this further, HASMCs shifted to the growth arrest medium were exposed to FRS2α or control shRNA lentiviruses in the presence or absence of the let-7b lentivirus. In agreement with the data presented above, HASMC FRS2α knockdown accelerated reversion to the contractile phenotype (FIG. 3E). The phenotype conversion, however, was effectively blocked by let-7 overexpression as demonstrated by decreased TGFβR1, SM-calponin, and SM-MHC expression and reduced Smad2 phosphorylation (FIG. 3E).

### Example 3: Activation of FGF and Loss of TGFβ Signaling in Human and Mouse Atherosclerotic Lesions

To examine the role played by FGF regulation of TGFβ signaling activity in SMCs in disease settings, the correlation between medial FGF and TGFβ signaling and the severity of atherosclerosis in samples of left main coronary arteries from forty-three patients was first evaluated (FIGS. 4A-4B). Table 1 summarizes clinical characteristics of this patient group. Immunostaining of serial left main coronary artery sections for SM α-actin and SM-MHC revealed decreased expression of these contractile SMC markers in the media of arteries from patients with moderate and severe coronary atherosclerosis compared to patients with No/mild disease (FIGS. 4C-4D), consistent with previous findings (Aikawa et al, 1995, Annals of the New York Academy of Sciences 748: 578-585; Aikawa et al, 1993, Circulation research 73: 1000-1012; Glukhova et al, 1988, Proc Natl Acad Sci USA 85: 9542-9546). At the same time, there was an increase in immunoreactivity for the phosphorylated form of FGFR1 in patients with moderate and severe CAD (coronary artery disease), implying an increase in FGF signaling (FIGS. 4E-4F). Yet there was no change in the medial FGFR1 expression levels (FIGS. 4G-4H).

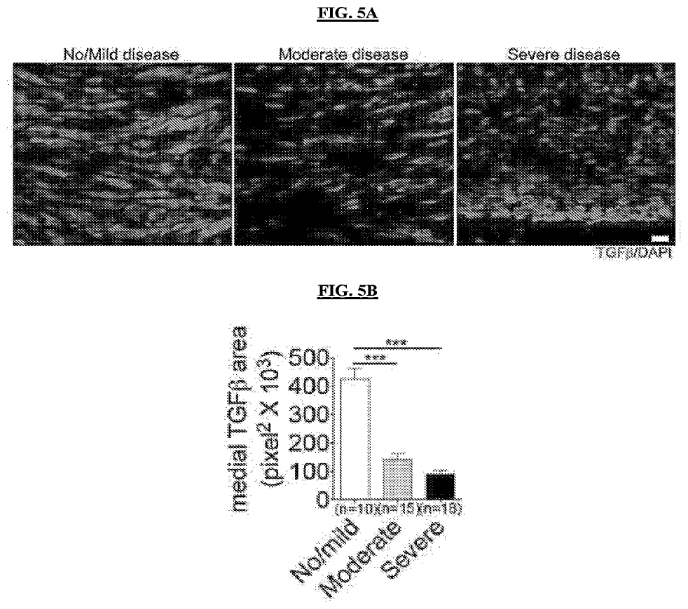

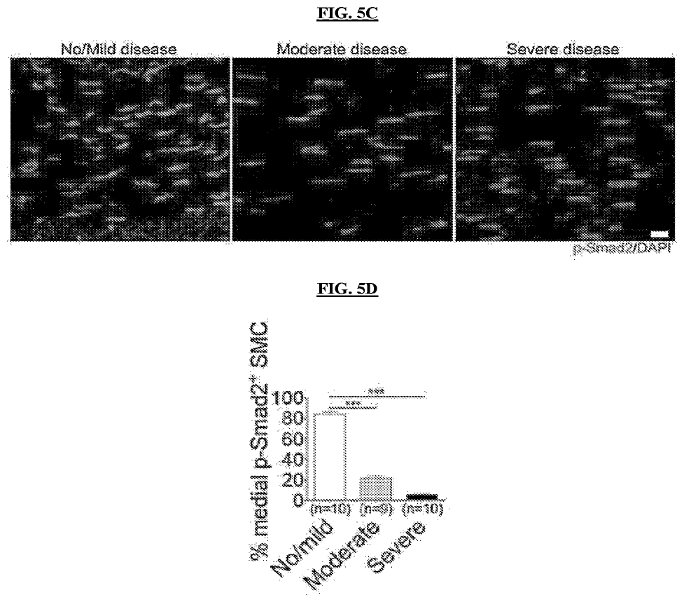

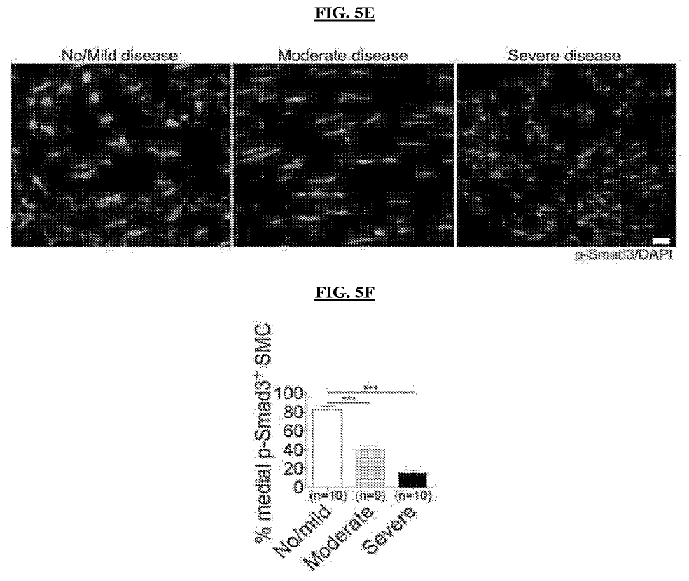

This activation of FGF signaling and the loss of smooth muscle contractile markers in advanced atherosclerotic lesions was accompanied by a decrease in TGFβ immunoreactivity in the media and the loss of p-Smad2 and p-Smad3 expression (FIGS. 5A-5F). Quantification of immunocytochemistry data from the left main coronary arteries of the entire patient cohort showed that while 84% of SMCs in patients with No/mild CAD demonstrated expression of p-Smad2 in the media of their coronary arteries, this was reduced to 21% in patients with moderate CAD and 6% in patients with severe CAD (FIG. 5D). Similarly, 83% of SMCs in patients with No/mild CAD demonstrated expression of p-Smad3 in the media of their coronary arteries, this was reduced to 41% in patients with moderate CAD and 16% in patients with severe CAD (FIG. 5F).

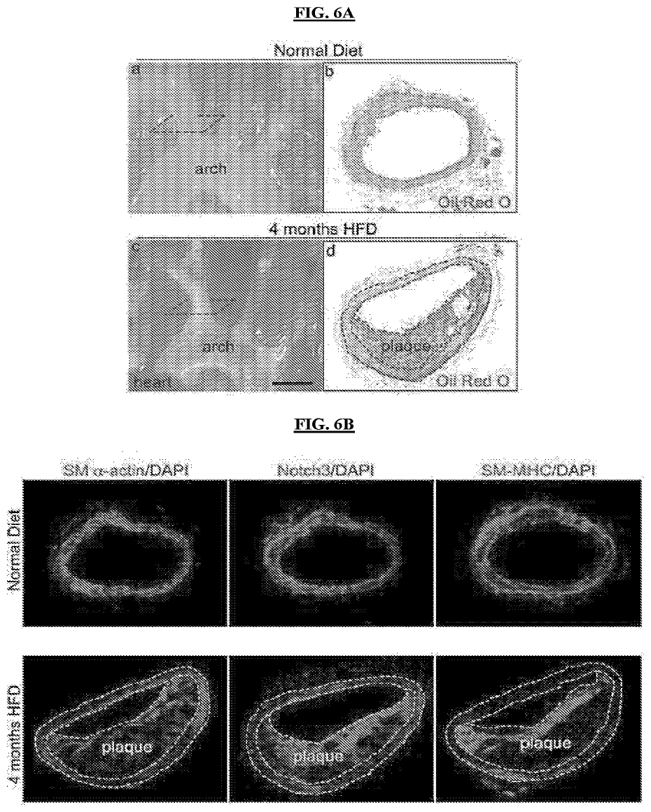

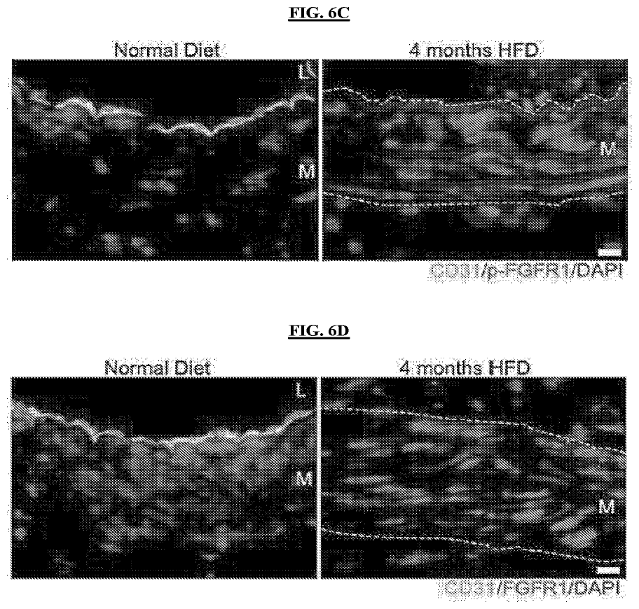

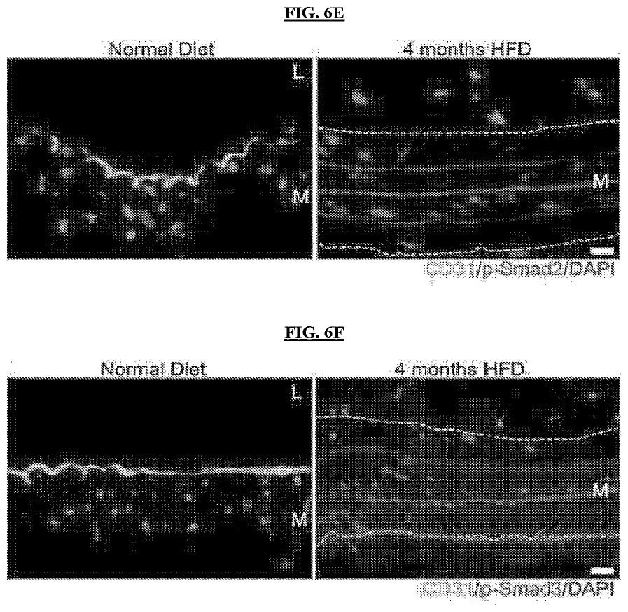

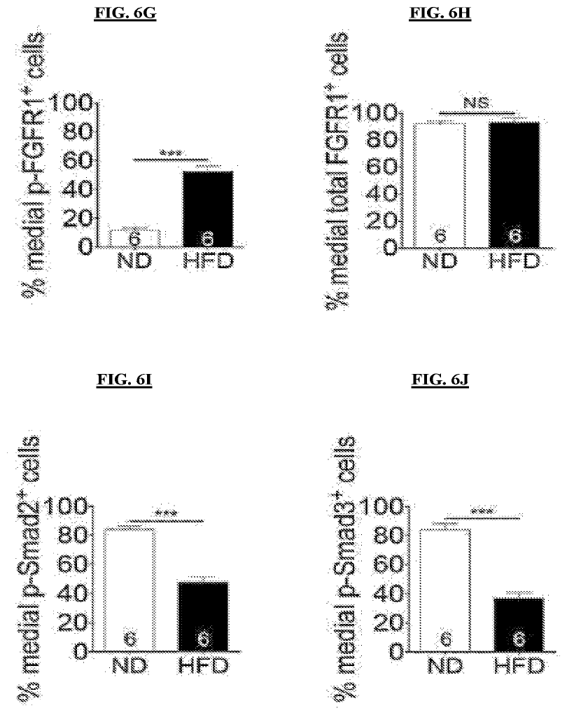

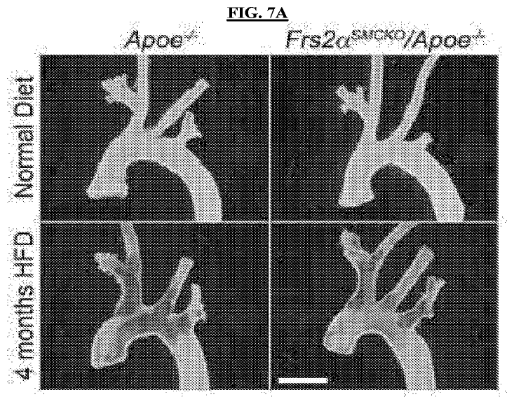

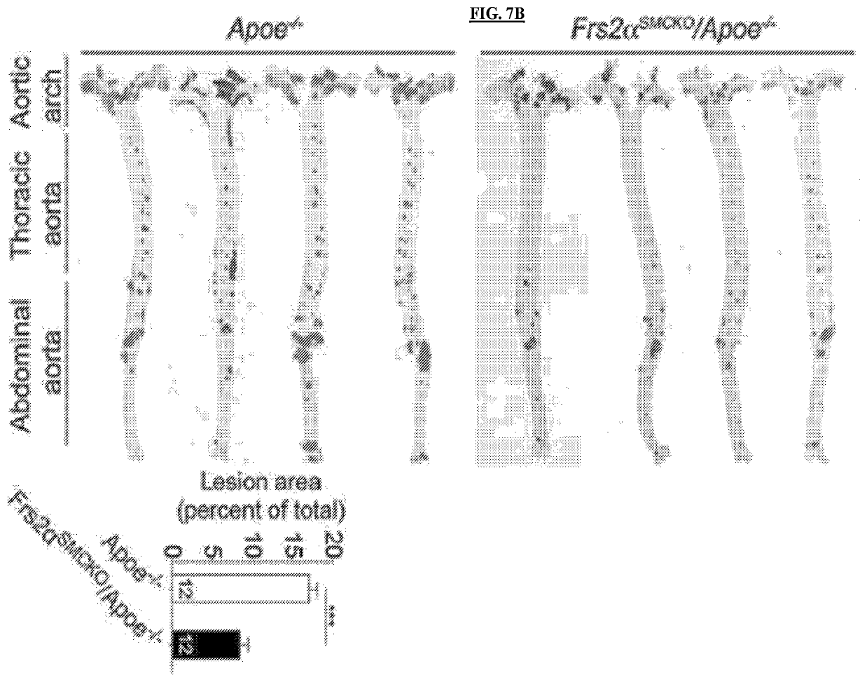

These findings were confirmed in an Apoe^{-/-} mouse model of atherosclerosis. After 16 weeks of high fat diet (HFD), medial SMCs in brachiocephalic artery atherosclerotic plaque had decreased expression of contractile SMC proteins compared to medial SMC of mice on a normal chow diet (FIGS. 6A-6B). This correlated with increased p-FGFR1 expression (FIGS. 6C and 6G) while total FGFR1 levels were unchanged (FIGS. 6D and 6H) and decreased p-Smad2, p-Smad3 activity (FIGS. 6E, 6F, 6I, and 6J).

### Example 4: Smooth Muscle-Specific Frs2α Deletion Reduces Atherosclerotic Lesion Growth

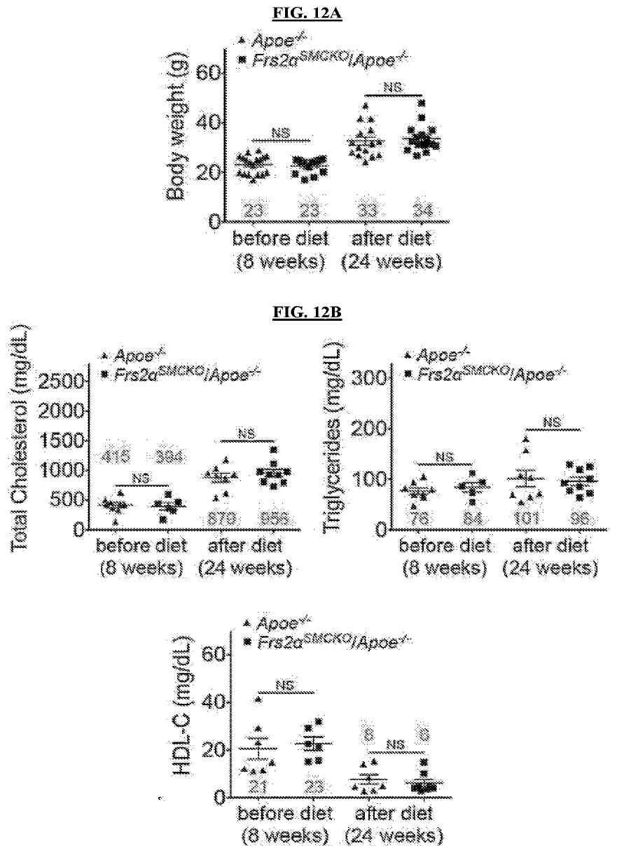

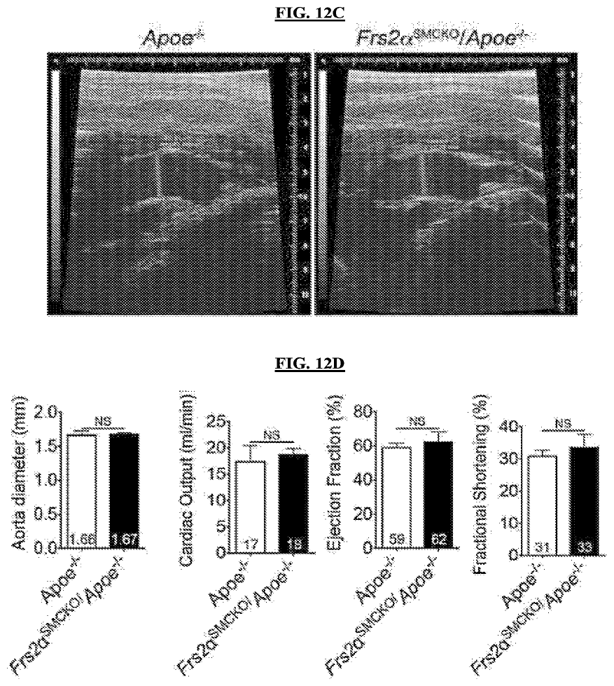

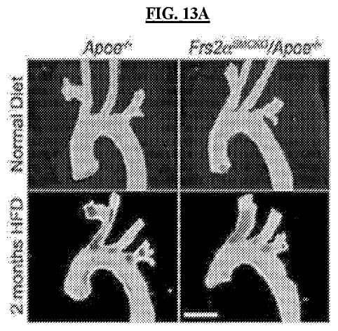

To further study the link between the loss of SMC FGF signaling and their phenotype modulation in vivo, mice with an SMC-specific Frs2α deletion (Frs2α SMCKO) using the SM22αCre line were generated (Holtwick et al, 2002, Proc Natl Acad Sci USA 99: 7142-7147). Frs2α^{SMCKO} mice were viable and born at the expected Mendelian frequency. Assessment of FRS2α expression levels in vascular tissue revealed a robust deletion of FRS2α in the aorta (FIGS. 11A-11C). There were no differences in the gross appearance of ascending or descending aorta between control and Frs2α^{SMCKO} mice (FIG. 11D) nor was there any difference in arterial wall thickness (elastic Van Gieson staining), smooth muscle contractile marker gene expression (SM α-actin, SM22a, Notch3), phosphorylated Smad2 (p-Smad2), and vascular density in the heart and skeletal muscle (FIGS. 11E-11H). Thus, the deletion of FRS2α per se did not alter the baseline structure of the normal vasculature.

To study the role of FGF signaling in the modulation of SMC phenotype during atherogenesis, Frs2α^{SMCKO} mice were crossed onto the atherosclerosis-prone Apoe^{-/-} background (Frs2α^{SMCKO}/Apoe^{-/-}). Male Frs2α^{SMCKO}/Apoe^{-/-} and Apoe^{-/-} littermates were placed on cholesterol-rich Western diet for eight or sixteen weeks at which point whole-mount Oil Red O staining was used to visualize the extent of aortic atherosclerotic plaques. There were no differences between these two groups with regard to body weight, total cholesterol, triglyceride, HDL-C levels, aorta diameter, or heart function (FIG. 12).

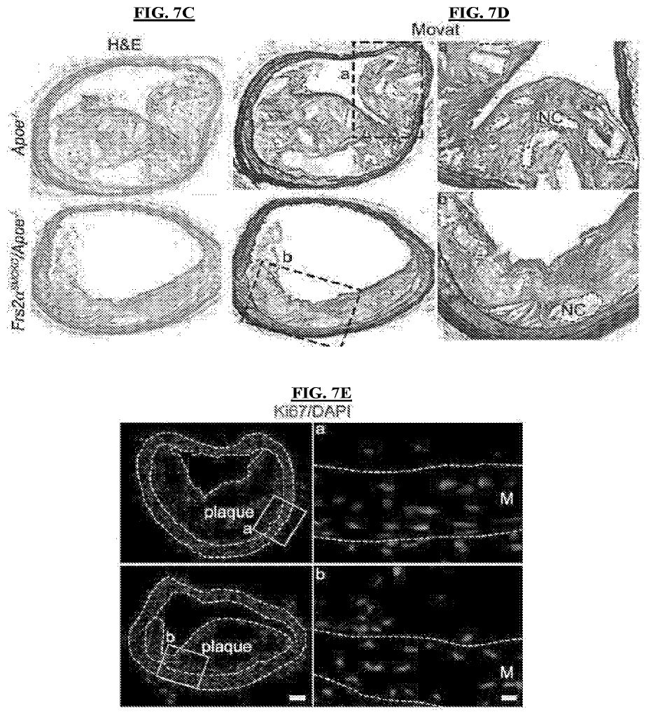

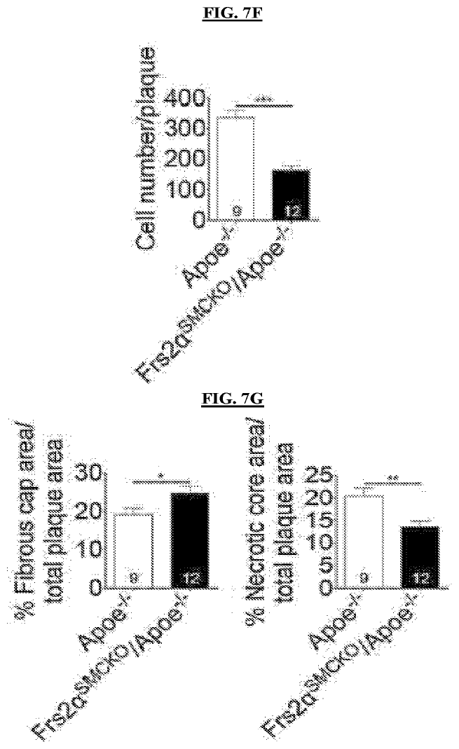

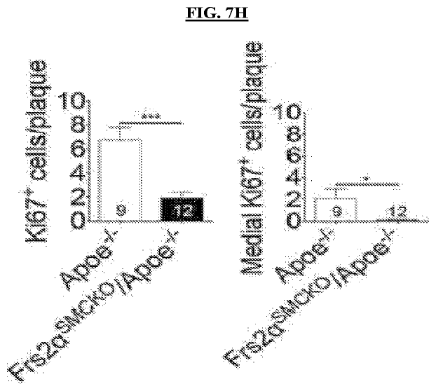

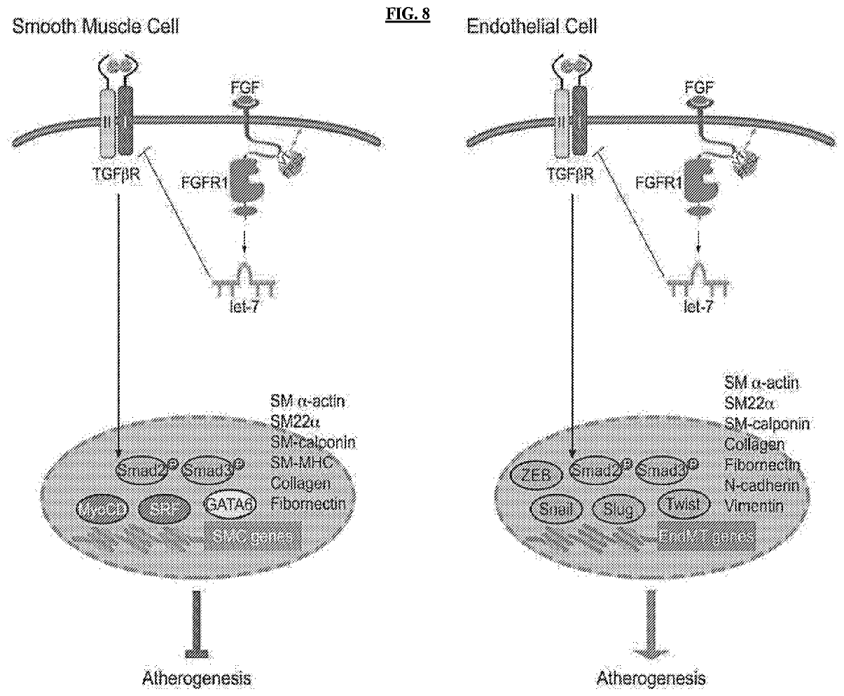

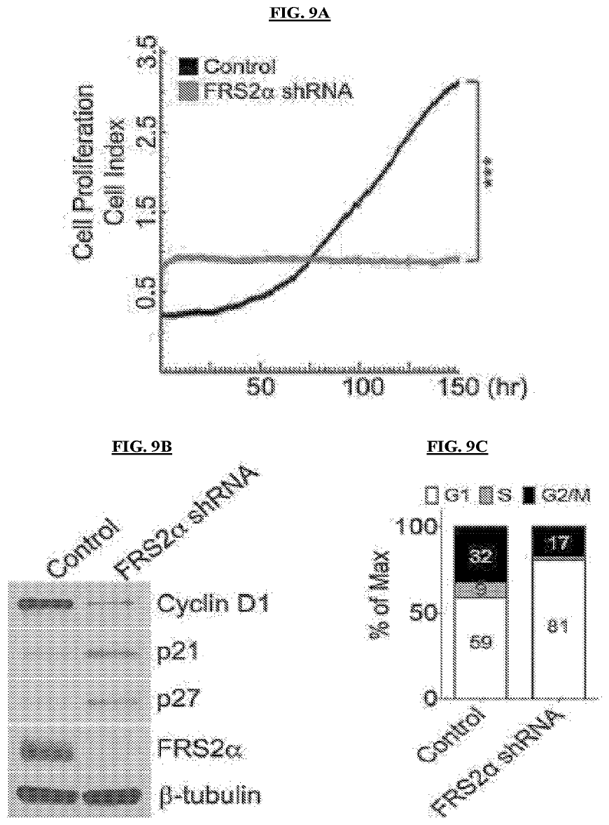

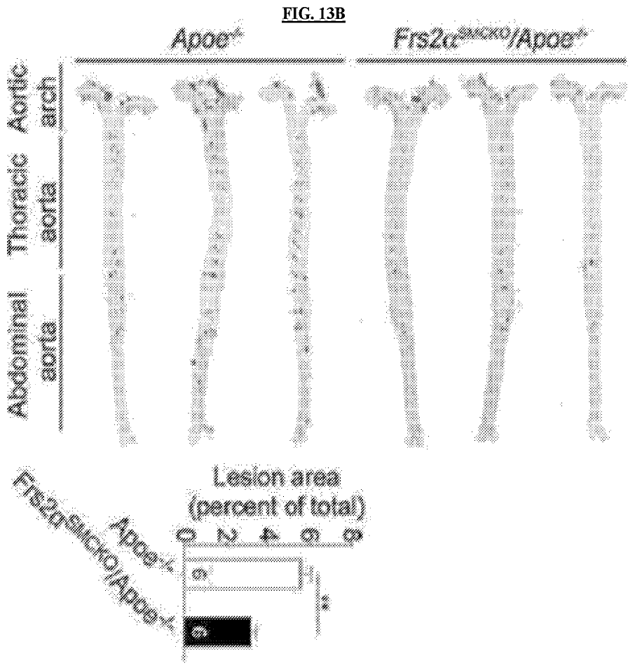

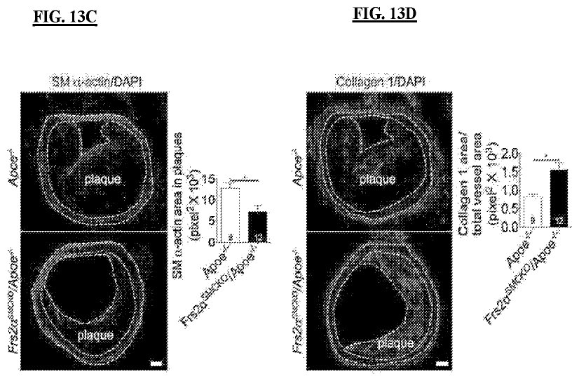

Aortas from Frs2α^{SMCKO}/Apoe^{-/-} and Apoe^{-/-} mice were examined after eight (FIGS. 13A-13B) or sixteen (FIGS. 7A-7B) weeks of high fat diet. In both cases, Frs2α^{SMCKO}/Apoe^{-/-} animals demonstrated much lower extent of the total aorta atherosclerotic plaque burden.

Notably, the progression of atherosclerosis was markedly reduced in Frs2α^{SMCKO}/Apoe^{-/-} mice compared to Apoe^{-/-} controls: by eight weeks there was a 43% decrease in the total aorta plaque size (5.57% in Apoe^{-/-} vs. 3.16% in Frs2α^{SMCKO}/Apoe^{-/-}) (FIG. 13B) and by sixteen weeks 54% decrease (17.24% in Apoe^{-/-} vs. 7.86% in Frs2α^{SMCKO}/Apoe^{-/-}) (FIG. 7B). Histochemical analysis of plaques showed a ˜50% reduction in plaque cellularity (335 cells/plaque in Apoe^{-/-} vs. 164 cells/plaque in Frs2α^{SMCKO}/Apoe^{-/-}) (FIGS. 7C and 7F).

Furthermore, Movat staining demonstrated that fibrous caps were thicker and necrotic core were smaller in Frs2α^{SMCKO}/Apoe^{-/-} compared to Apoe^{-/-} mice (FIGS. 7D and 7G). Finally, Ki67 staining demonstrated reduced proliferation rate in plaque as well as media cells (FIGS. 7E and 7H). All of these findings are consistent with a more stable plaque phenotype. Consistent with these changes in plaque cellularity and fibrous cap size, there was a decrease in the plaque SM α-actin area (12.82 in Apoe^{-/-} vs. 7.28 in Frs2α^{SMCKO}/Apoe^{-/-}) and increased collagen deposition (0.83 in Apoe^{-/-} vs. 1.56 in Frs2α^{SMCKO}/Apoe^{-/-}) (FIGS. 13C-13D).

### Example 5: Suppression of Endothelial Cell TGF$ Signaling in an Atherosclerosis Mouse Model Reduced Formation of Atherosclerotic Lesion

It was previously shown that FGF regulated TGFβ signaling via let-7 miRNA (Chen et al., 2012, Cell Reports 2: 1684-1696). Further, it was demonstrated that endothelial to mesenchymal transition drives atherosclerosis (Chen et al., 2015, Journal of clinical investigation 125: 4529-4543). In this study, blocking endothelial cell TGFβ signaling was examined to determine whether or not this would reduce atherosclerotic lesions.

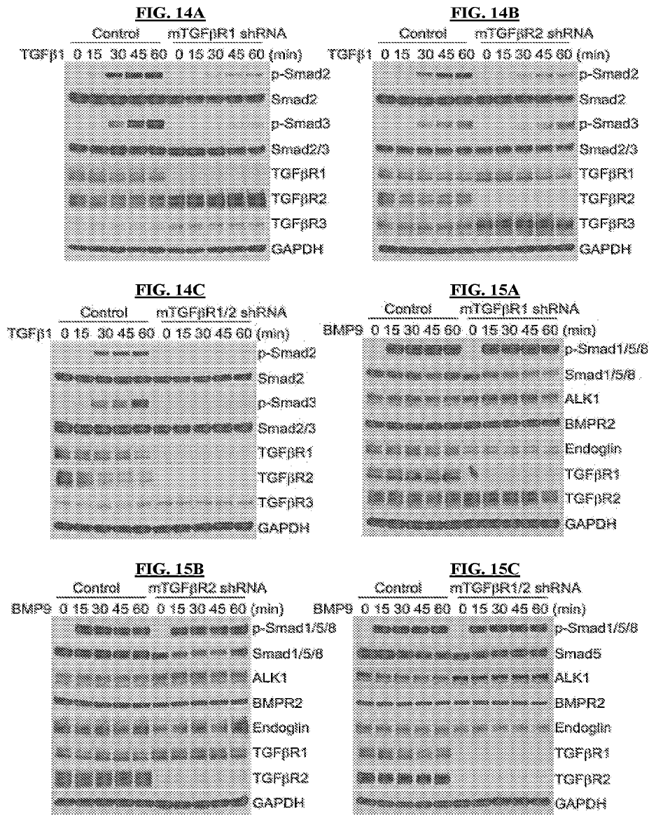

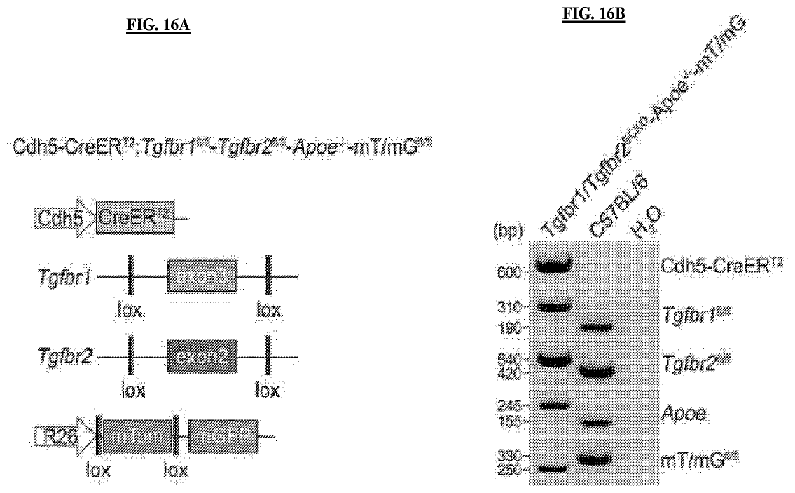

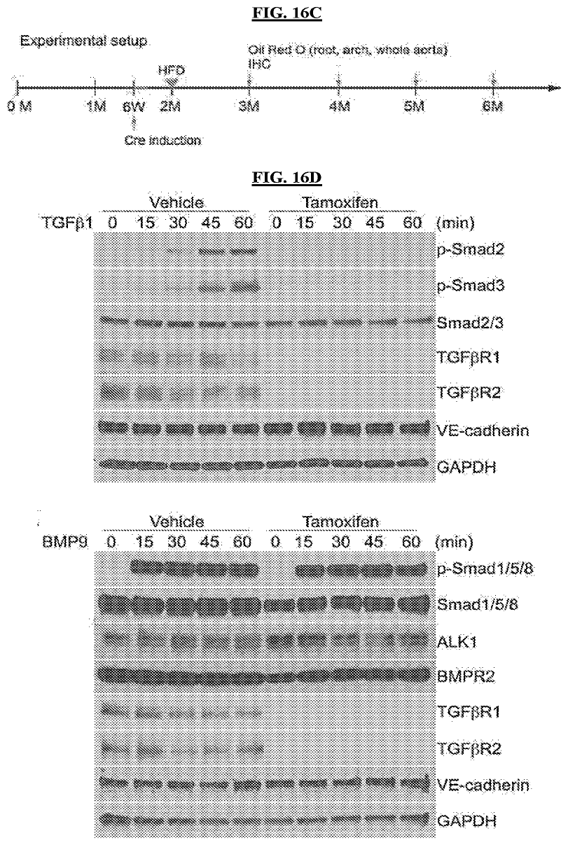

Knockdown of TGFβR1 and/or TGFβR2 suppressed TGFβ signaling activity, but not BMP signaling (FIGS. 14A-14C; FIGS. 15A-15C). Thus, to block TGFβ signaling in endothelial cells, mice with an inducible endothelial specific knockout of TGFβ receptors 1 and 2 (TGFβR1 and TGFβR2) were generated and crossed on the Apoe^{-/-} background to induce atherosclerosis susceptibility. A mTmG strain was also generated to fate-map endothelial cells (FIGS. 16A-16B). Both TGFβ receptors were deleted as a knockdown of either TGFβR1 or TGFβR2 did not fully abolish TGFβ-driven Smad2 and Smad3 phosphorylation (FIG. 14A-14C). The resultant mutant mice (Cdh5CreER^{T2}; Tgfbr1^{fl/fl}; Tgfbr2^{fl/fl}; Apoe^{-/-}; mT/mG^{fl/fl}), hereby referred to as Tgfrbr^{iECKO}/Apoe^{-/-}, with littermate controls (absent Cdh5CreER^{T2}, mice without Tgfbr1 or Tgfbr2 loci and non-induced mice) were used for subsequent experiments. Testing of primary endothelial cells (tagged with eGFP) isolated from the Tgfrbr^{iECKO}/Apoe^{-/-} mice showed that activation of the Cdh5CreER^{T2} gene at six weeks of age led to a complete deletion of both targeted Tgfbr genes (FIGS. 16C-16D). This fully blocked TGFβ signaling while preserving BMP signaling (FIG. 16D).

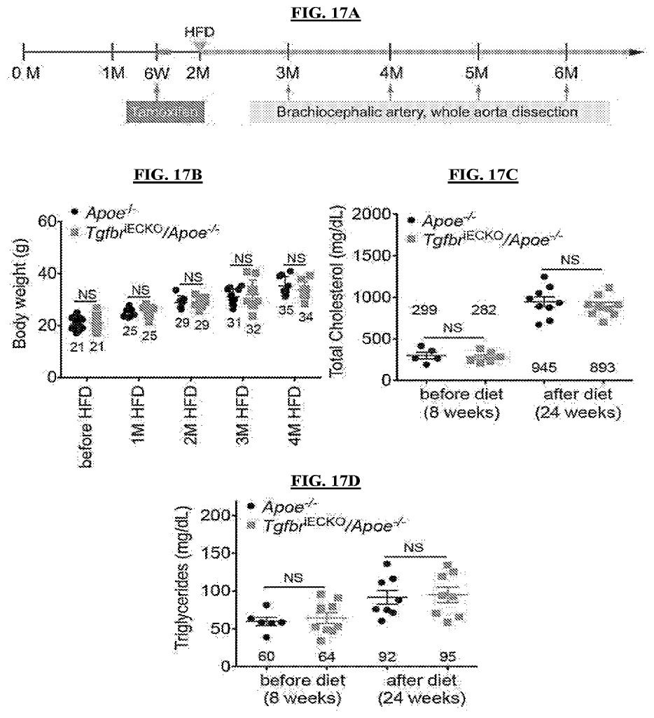

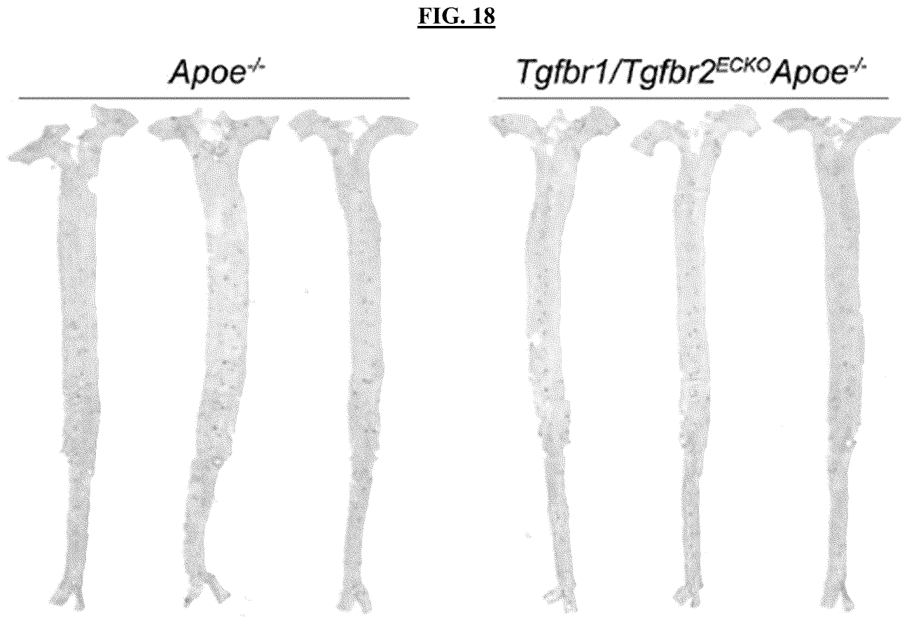

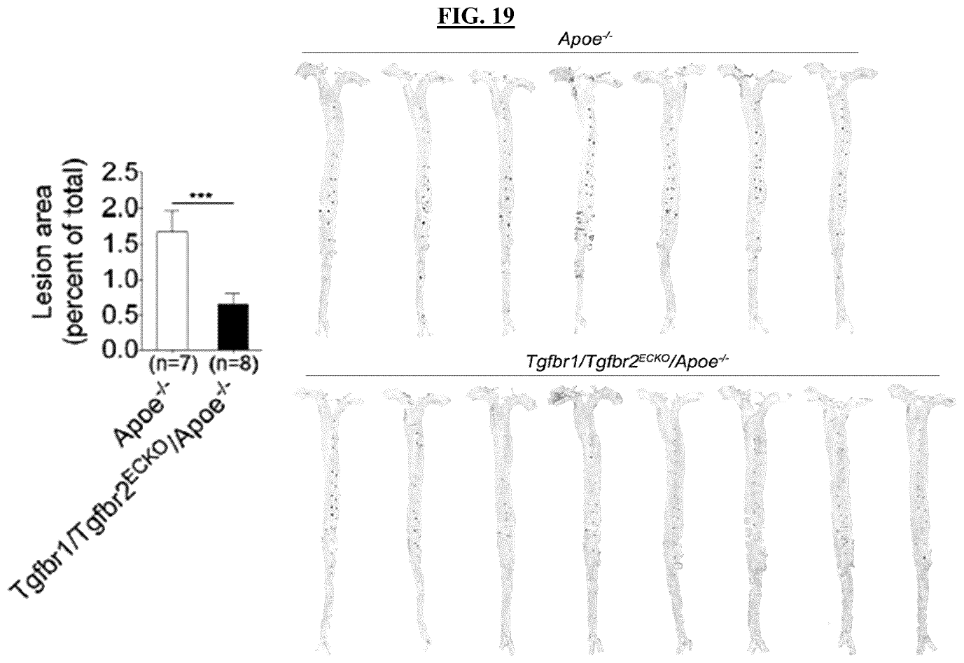

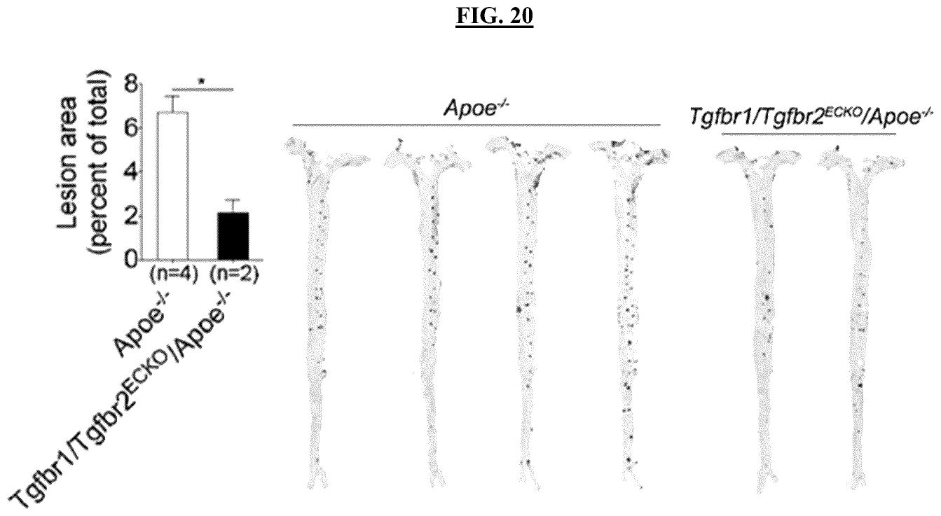

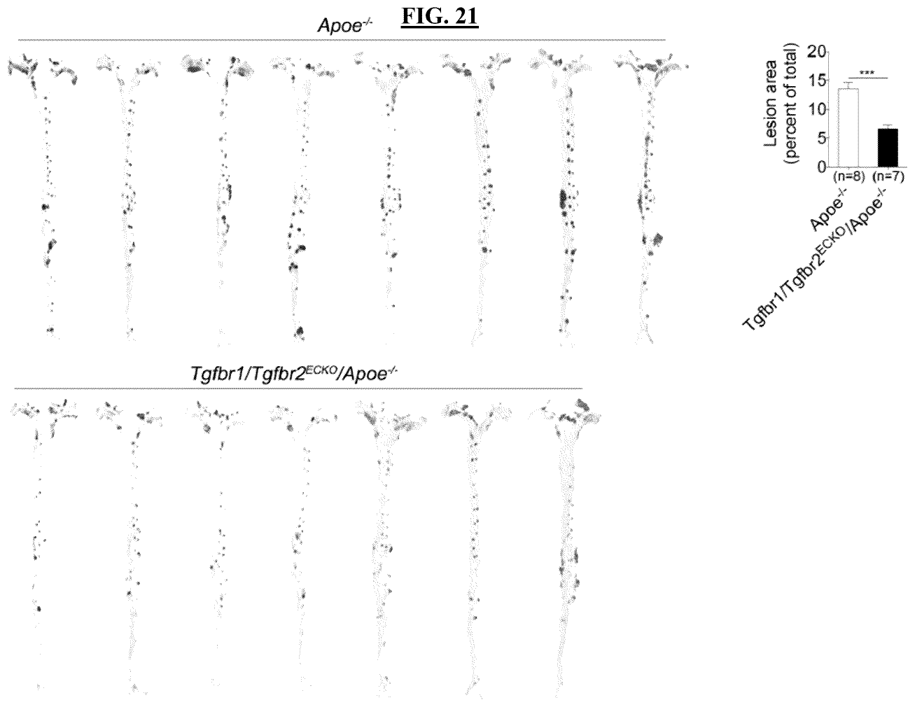

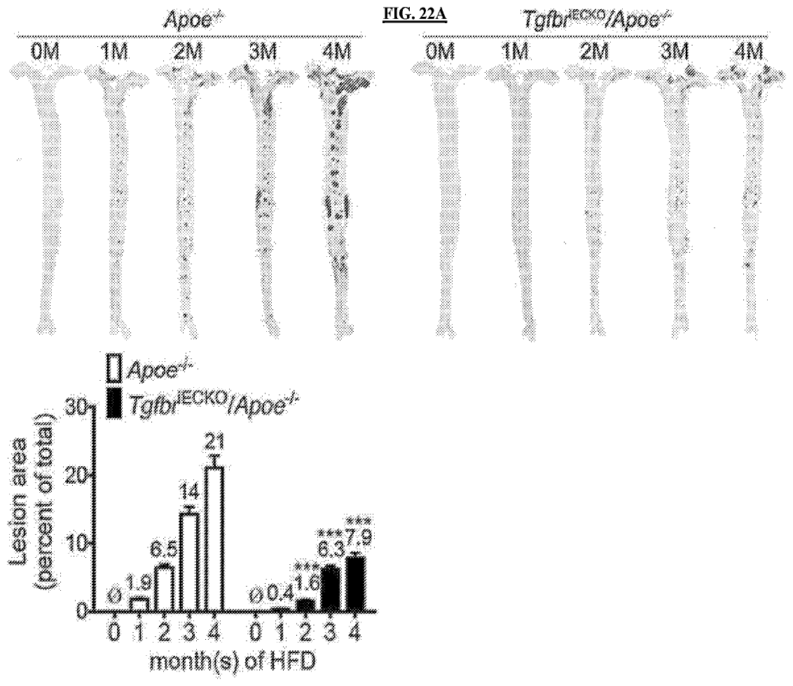

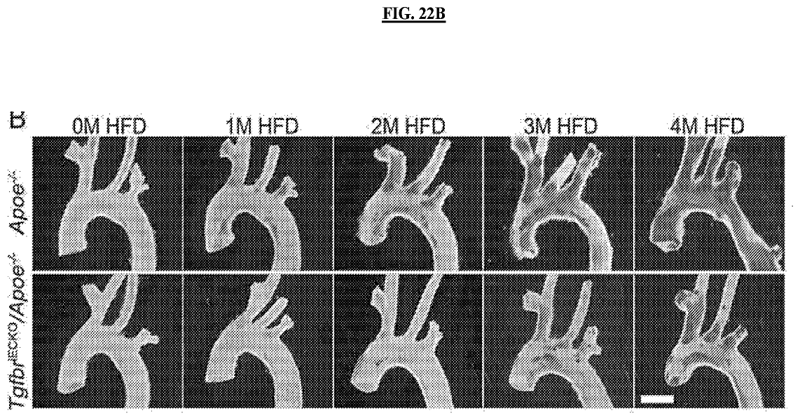

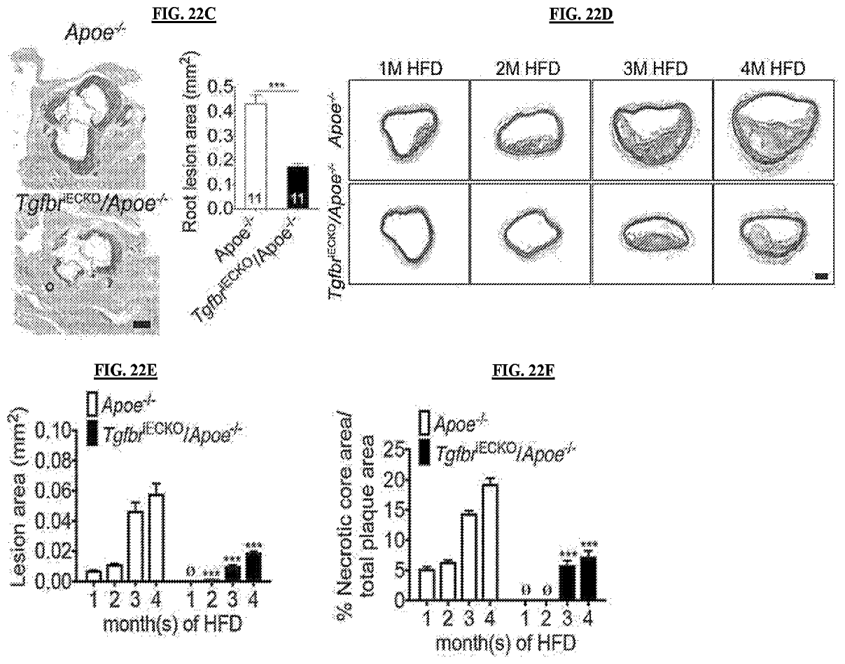

Tgfrbr^{iECKO}/Apoe^{-/-} and littermate control mice were placed on a high fat diet (HFD) 2 weeks after induction of Tgfbr1/2 excision (FIG. 17A). The dietary intervention resulted in an increase in body weight, total serum cholesterol and triglycerides that was similar in both groups (FIGS. 17B-17D). No plaque development was observed in mice fed with a normal diet at 8 weeks old (FIG. 18). Serial analysis of whole aortas and aortic arches using Oil-Red-O staining demonstrated a significantly delayed onset and reduced extent of lipid deposition in Tgfrbr^{iECKO}/Apoe^{-/-} mice (FIG. 19, FIG. 20, FIG. 21 and FIGS. 22A-22B). Quantitative assessment showed a 55%-79% reduction in the total aorta area of Oil-Red-O staining in aortas of Tgfrbr^{iECKO}/Apoe^{-/-} mice over this time course (FIGS. 22A-22B). Examination of Oil-Red-O-stained aortic root cross-sections showed a 60% reduction in the plaque area after 4 months of HFD (FIG. 22C).

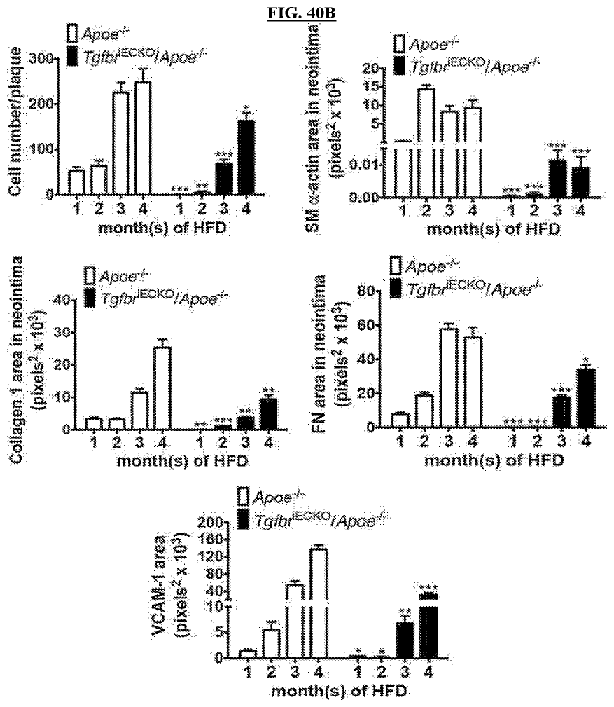

To study the effect of TGFβ receptors deletion on the composition and size of atherosclerotic plaques, brachiocephalic arteries from both groups of mice, sacrificed at monthly intervals, were serially sectioned. Histological examination demonstrated a marked reduction in the size of the plaque, a reduction in its necrotic core area, as well as a decrease in plaque cellularity (FIGS. 22D-22F, and FIGS. 40A-40B). The differences were most pronounced early in the time course: after 1 month of HFD, half of the Apoe^{-/-} mice exhibited pathological intimal thickening and half had evidence of intimal xanthomas while all Tgfrbr^{iECKO}/Apoe^{-/-} mice appeared normal. Fibrous cap atheromas were evident after 2 months of HFD in Apoe^{-/-} mice, but they did not appear in Tgfrbr^{iECKO}/Apoe^{-/-} mice until a month later. Even after 4 months of HFD, fibrous cap atheromas were present in only a half of Tgfrbr^{iECKO}/Apoe^{-/-} mice (FIG. 22D) (Lutgens et al, 2010, The Journal of experimental medicine 207, 391-404; Virmani et al, 2000, Arterioscler Thromb Vasc Biol 20, 1262-1275).

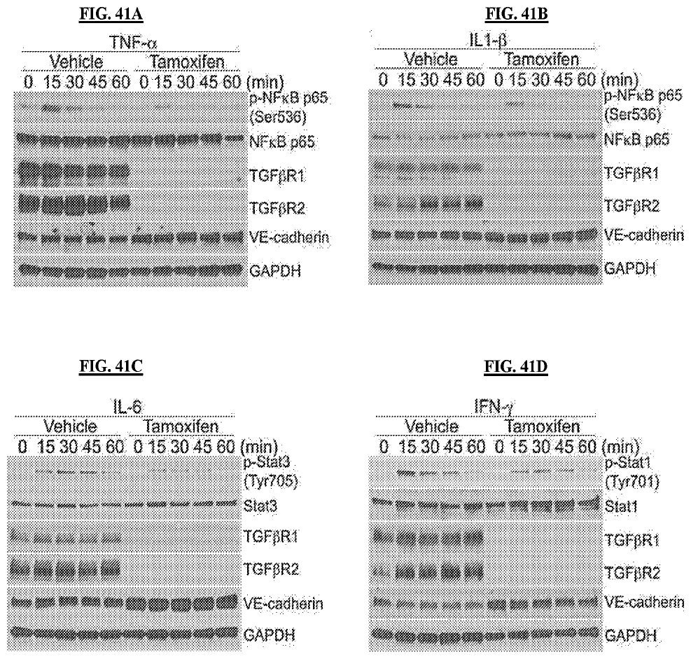

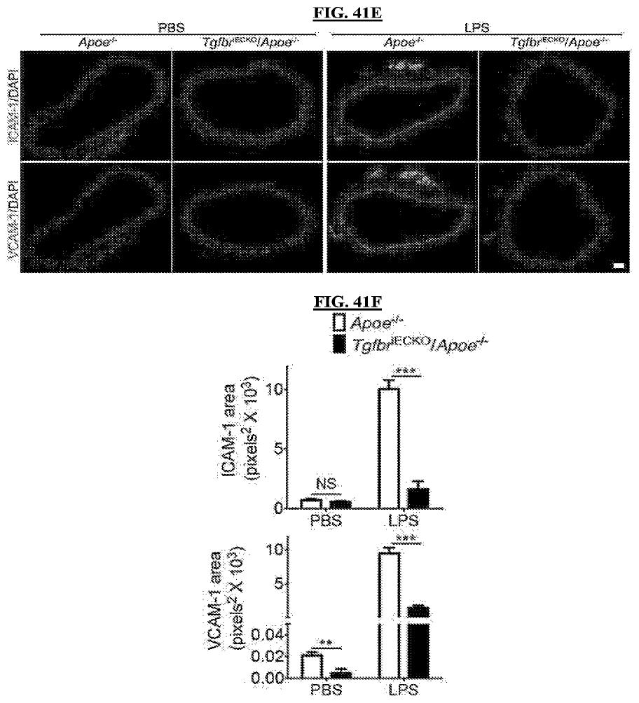

Staining with an anti-αSMA Ab demonstrated a decrease in the number of neointimal αSMA⁺ cells (FIG. 40A-40B) and reduced neointimal expression of collagen, findings consistent with decreased EndMT. In addition, there was a reduction in fibronectin deposition and a decrease in endothelial VCAM-1 expression (FIGS. 40A-40B), indicating a reduction in “inflammatory” state of the endothelium. To further test the effect of inhibition of endothelial TGFβ signaling on its response to inflammatory mediators, primary endothelial cells from Tgfrbr^{iECKO}/Apoe^{-/-} and Apoe^{-/-} mice were treated with different inflammatory mediators (FIGS. 41A-41D).

The knockout of TGFβ receptors led to a significant decrease in NFκB phosphorylation in response to TNF-α and IL1-β (FIG. 41A-41-B), Stat3 phosphorylation in response to IL-6 (FIG. 41C), and Stat1 phosphorylation in response to IFN-γ (FIG. 41D). The decreased responsiveness of Tgfrbr^{iECKO}/Apoe^{-/-} mice to inflammatory stimuli was confirmed in vivo: staining of the thoracic aortic endothelium demonstrated a profound reduction in ICAM-1 and VCAM-1 expression after LPS injection compared to littermate controls (FIGS. 41E-41F).

In sum, this study established that endothelial TGFβ receptor knockout mice developed smaller atherosclerotic lesions than wild-type Apoe mice. The results of this study provide the first genetic evidence of pro-atherogenic endothelial cell TGFβ signaling in atherosclerosis.

### Example 6: Delivery of Let-7 to Endothelial Cells Using 7C1 Nanoparticle Reduced Atherosclerotic Lesion Growth and Formation

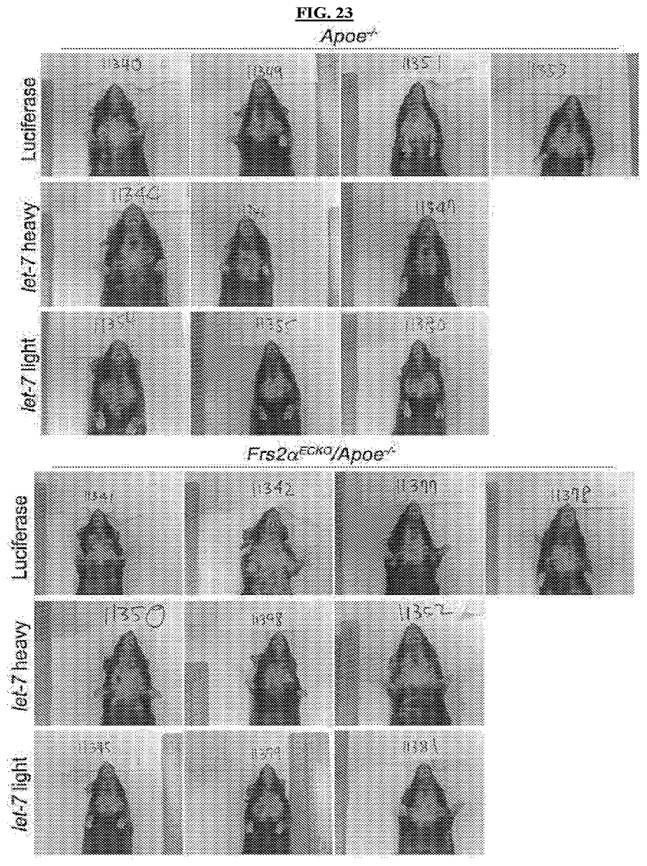

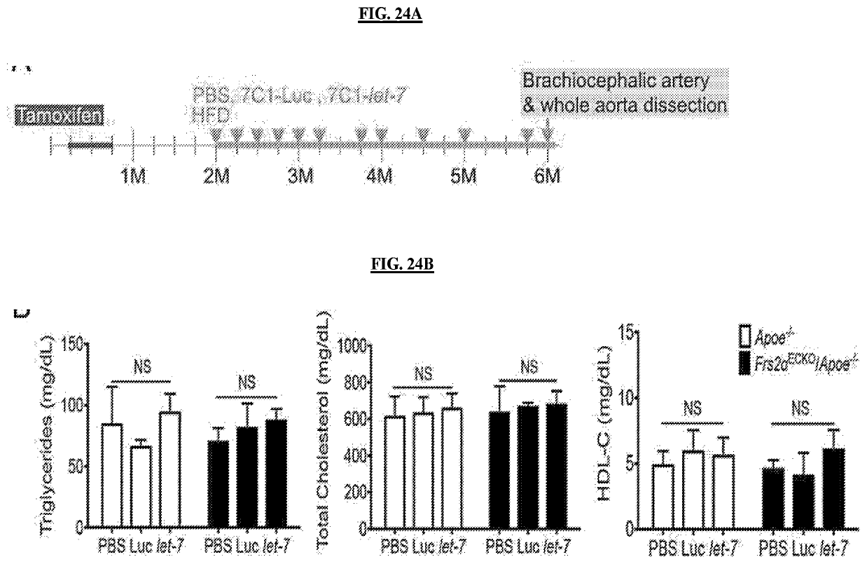

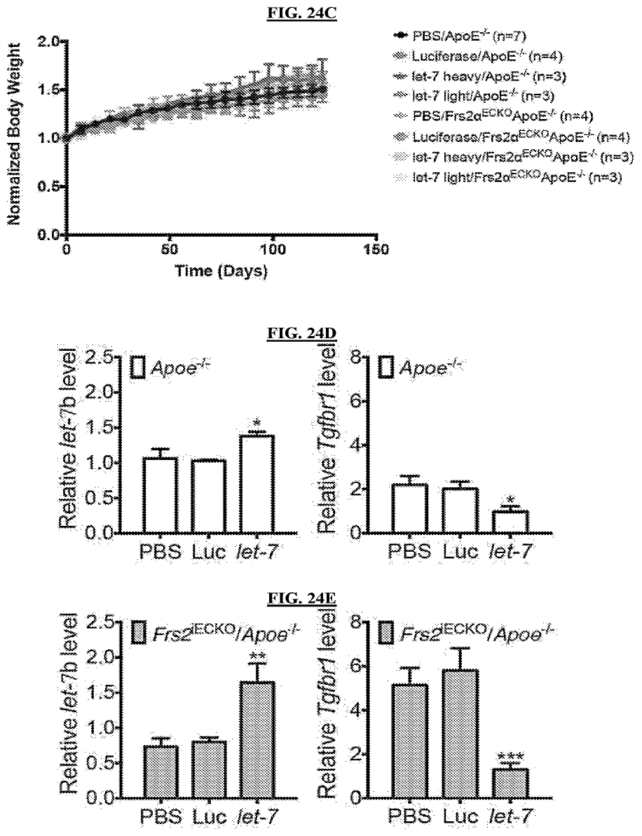

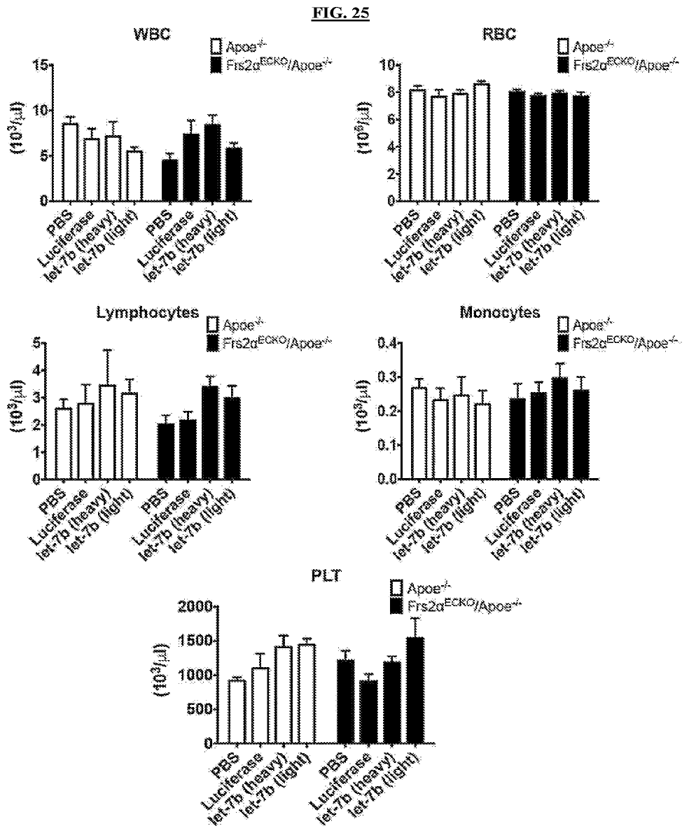

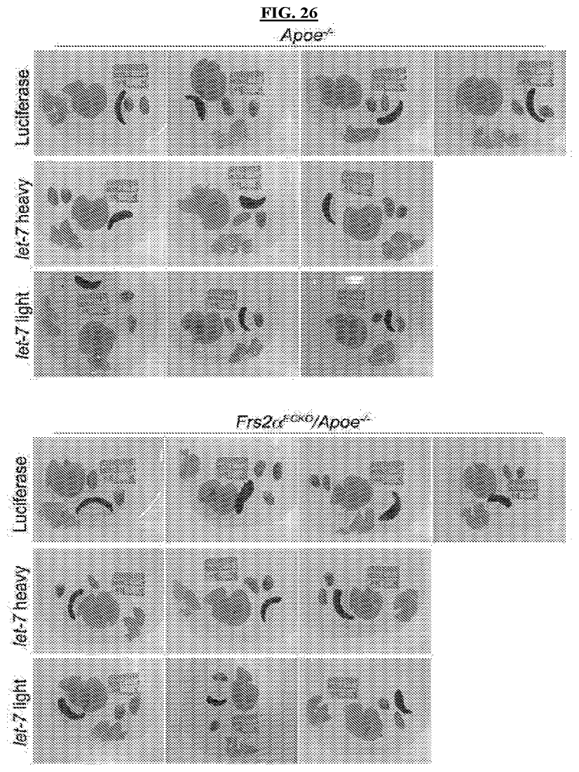

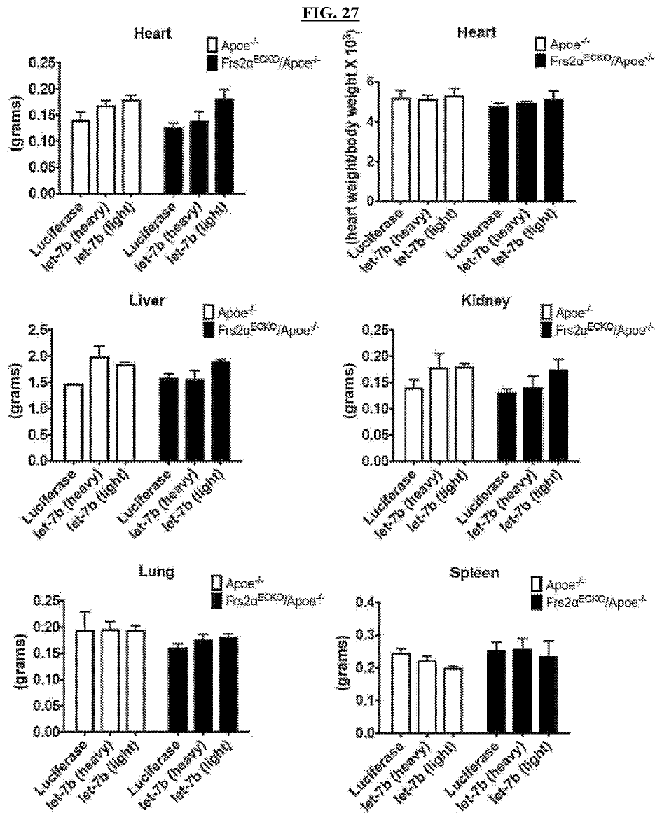

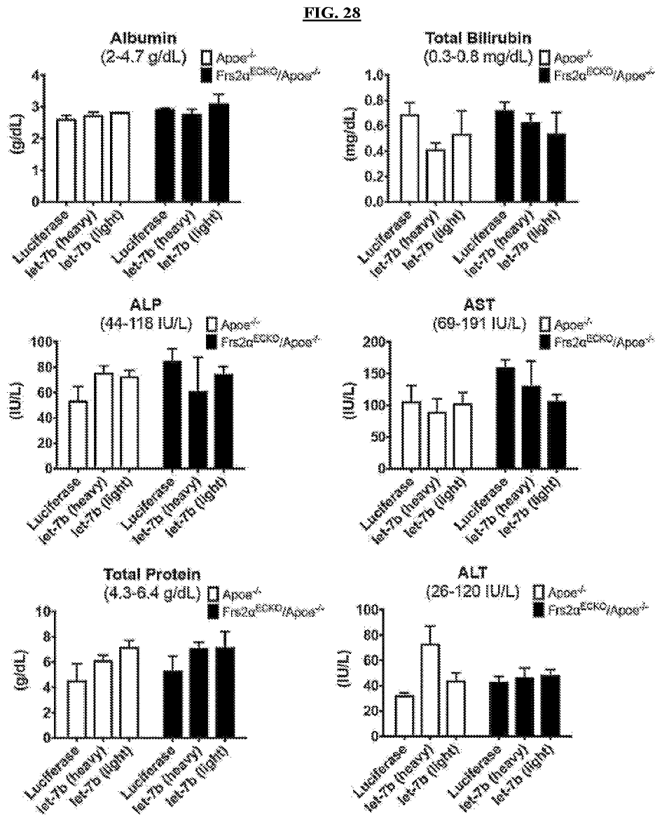

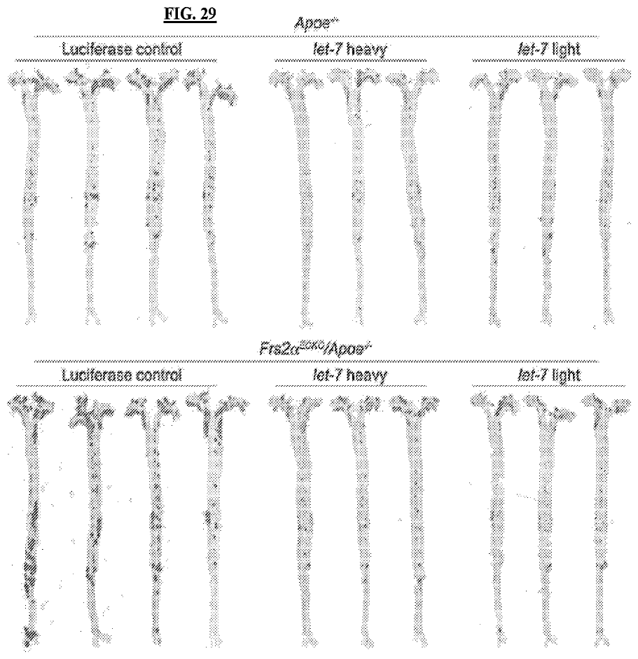

A decline in let-7 miRs expression has been previously linked to activation of TGFβ signaling (Chen et al., 2012, Cell reports 2: 1684-1696; Chen et al, 2014 Science signaling 7, ra90). In another study, the effect of delivery of let-7 miRNA to endothelial cells on reduction of atherosclerosis was investigated, thus it was tested if restoration of endothelial let-7 levels would reverse TGFβ activation and reduce atherosclerosis. Apoe null (Apoe^{-/-}) and Frs2α^{iECKO}/Apoe^{-/-} mice were administered with a luciferase control, let-7 heavy (mi-let-7b_{H}), and let-7 light (mi-let-7b_{L}) using a nanoformulation (7C1 nanoparticle) for selective delivery to endothelial cells. A chemically modified let-7b miR was packaged into 7C1 nanoparticles (Dahlman et al., Nat Nanotechnol. 2014 August; 9(8): 648-655) and used for in vivo delivery in Apoe^{-/-} and Frs2α^{iECKO}/Apoe^{-/-} mice that demonstrate enhanced atherosclerosis. 7C1 intravenous injections were performed 12 times within 4 months. Measurements of the following were taken from mice in each group: body weight, complete blood counts, skin pictures, serum lipid profiles, organ weight, and whole aorta oil red-O staining and quantification (FIGS. 24C, 25, 23, 24B, 27, and 29). It was observed that let-7 injected mice did not scratch around their neck (FIG. 23), mice in all groups gained weight (FIG. 24C), their blood cell counts and serum lipid profile were all within normal range (FIGS. 25 and 24B) and all their organs appeared normal (FIGS. 26 and 27). Furthermore, a slight increase in total protein level and a reduction in lesion formation were observed in the let-7 injected group of mice (FIGS. 28 and 29).

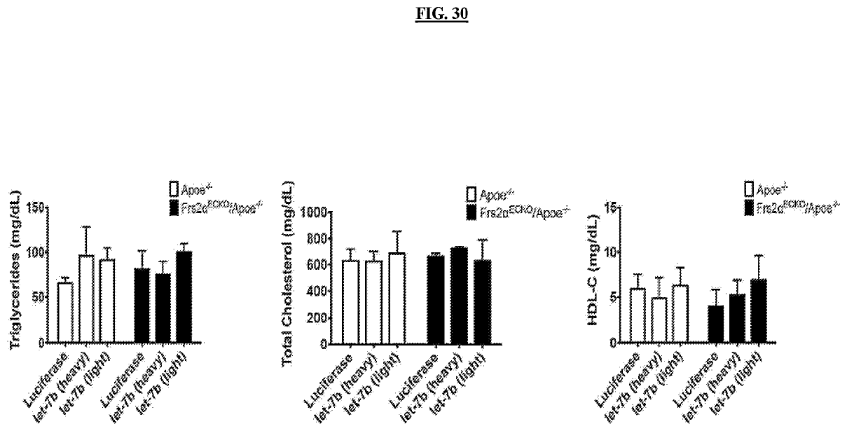

FIG. 30 shows that triglyceride, cholesterol, and high density lipoprotein (HDL) levels in Apoe^{-/-} and Frs2α^{iECKO}/Apoe^{-/-} mice injected with let-7 miRNA (let-7 heavy, let-7 light) or a luciferase control were all similar. This finding is important because it shows the reduced plaque lesions in let-7 injected groups was not due to reduced triglyceride and/or reduced cholesterol in the blood.

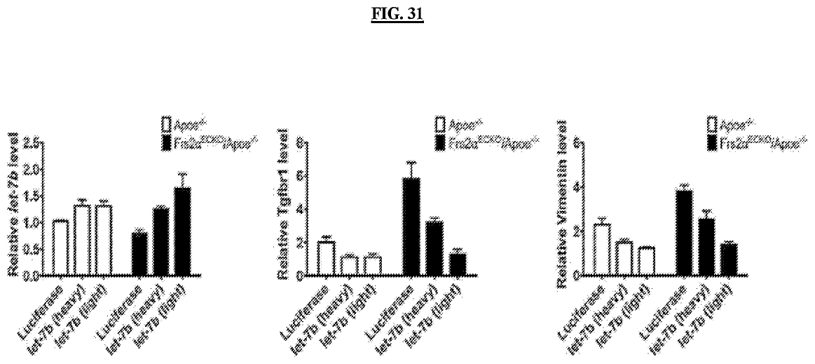

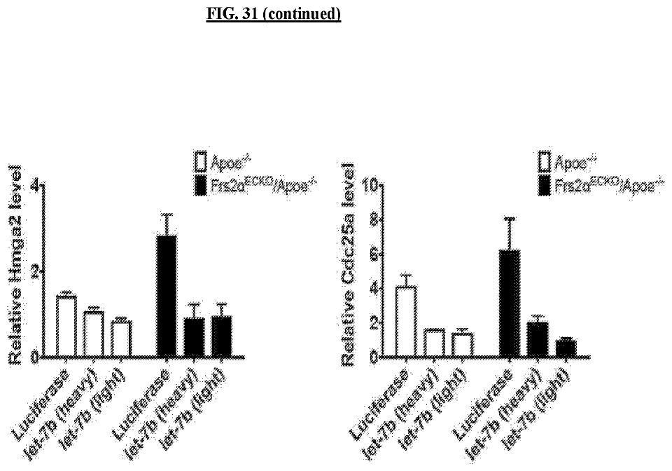

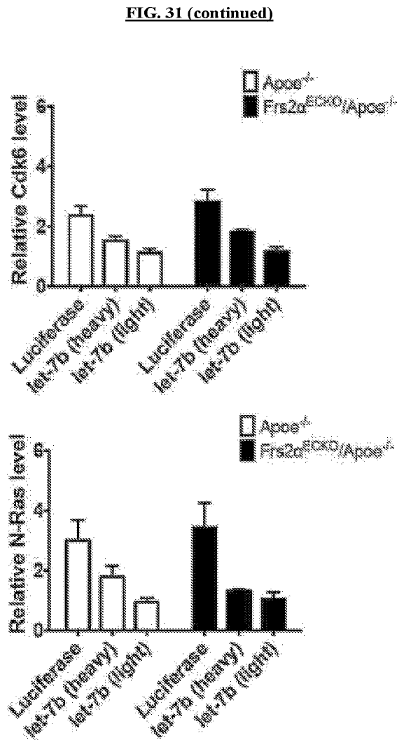

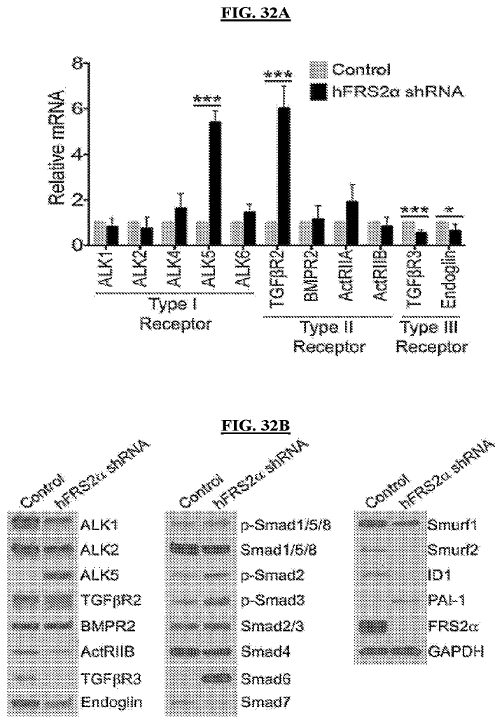

Intravenous therapy was initiated at the same time as the switch to the HFD and continued at intervals as shown in FIG. 24A. The let-7b miR administration had no effect on serum triglycerides, total cholesterol or HDL cholesterol levels (FIG. 24B). The normal weight gain seen in mice on the HFD was not affected (FIG. 24C), and there were no abnormalities in any of the biochemical parameters tested, including liver function tests (FIG. 28). Analysis of primary endothelial cells from the lungs of Apoe^{-/-} and Frs2α^{iECKO}/Apoe-/mice showed increased expression of let-7b miRNA, a consequent decrease in Tgfbr1, and let-7/TGFβ downstream gene expression (FIGS. 24D, 24E, and 31).

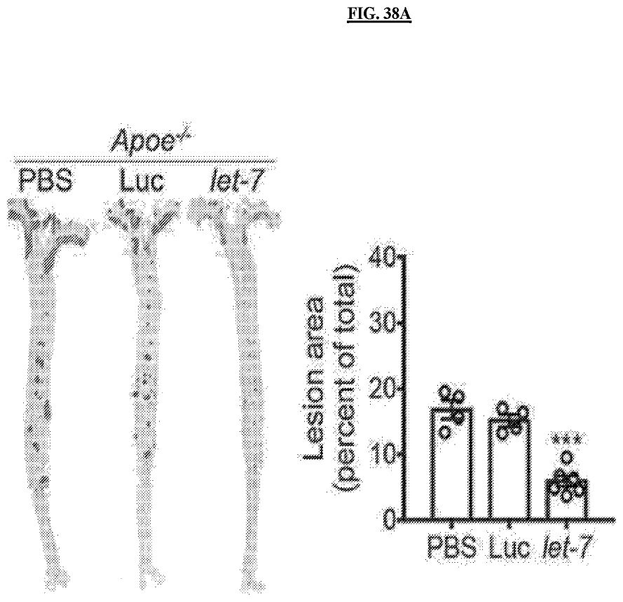

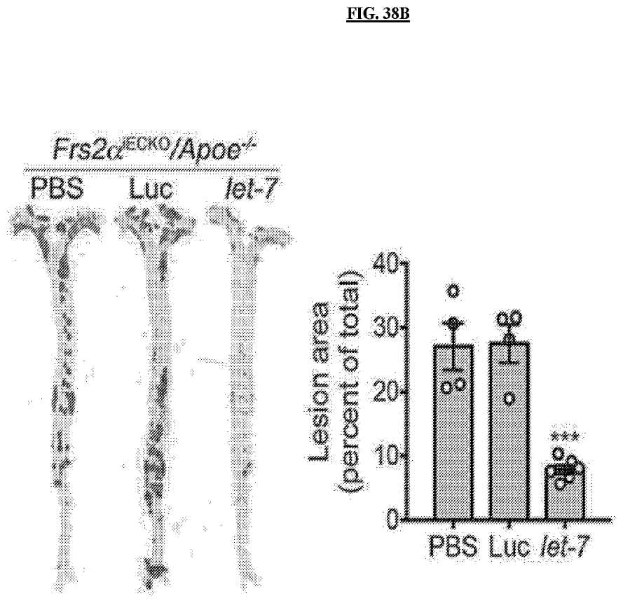

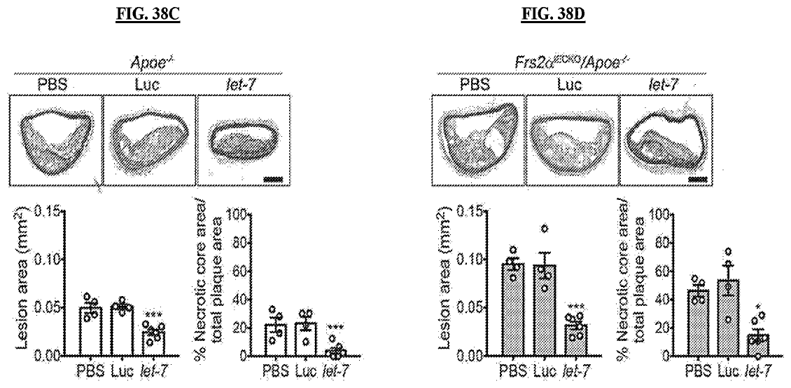

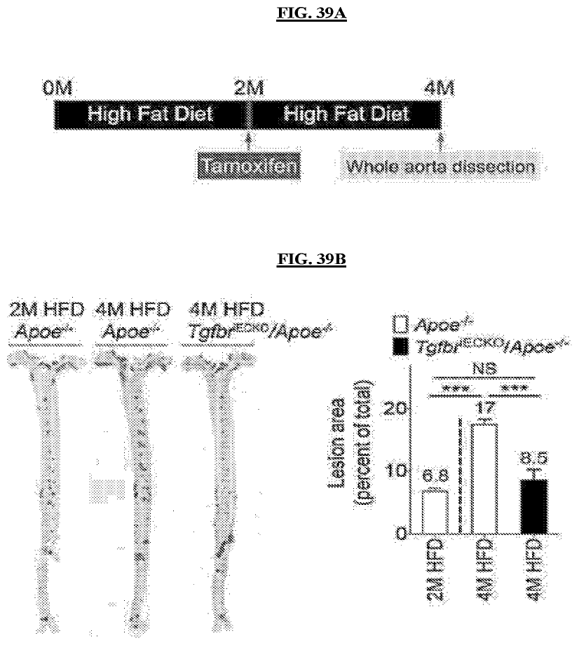

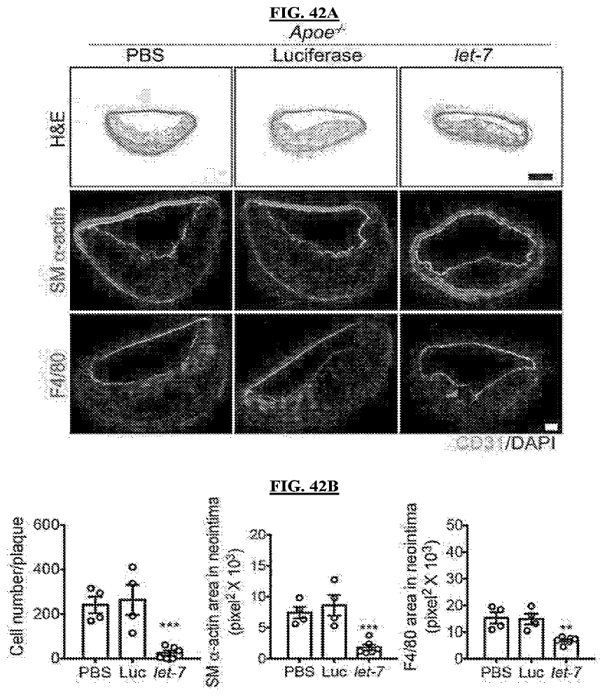

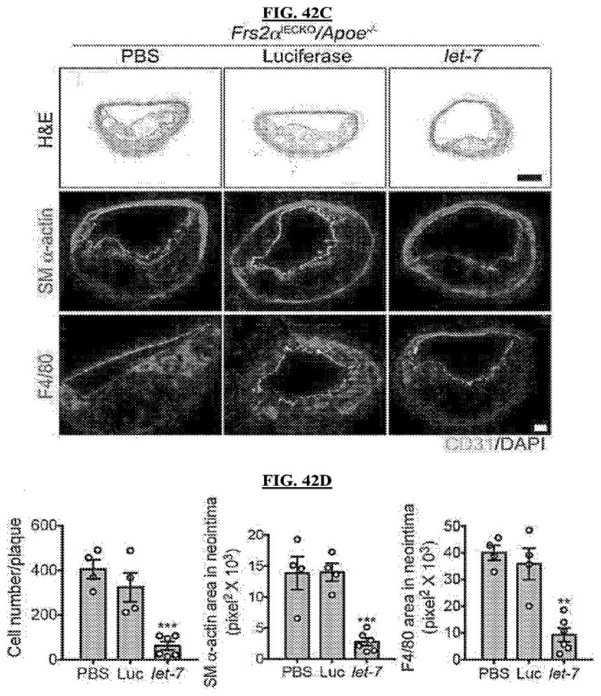

Examination of aortas of Apoe^{-/-} and Frs2α^{iECKO}/Apoe^{-/-} mice treated with let-7b miR 7C1 nanoparticles showed a 61% (Apoe^{-/-}) and 71% (Frs2α^{iECKO}/Apoe^{-/-}) reduction in Oil-red-O staining compared to control mice (FIGS. 38A-38B). Analysis of serial brachiocephalic artery sections (Movat staining) confirmed these finding: let-7 miR administration led to a significant reduction in the plaque area (50% in Apoe^{-/-} and 66% in Frs2α^{EC}KO/Apoe^{-/-} mice) and decrease in the necrotic core size (83% and 73%, respectively; FIGS. 38C-38D) that were similar to that seen in the Tgfrbr^{iECKO}/Apoe^{-/-} mice. Furthermore, let-7b therapy led to a decrease in the plaque cellularity and the number of neointimal αSMA⁺ cells and F4/80⁺ macrophages (FIGS. 42A-42D). Thus, endothelial-targeted nanoparticles loaded with let-7b miR achieved the same functional result as the deletion of endothelial TGFβR1 and TGFβR2 genes.

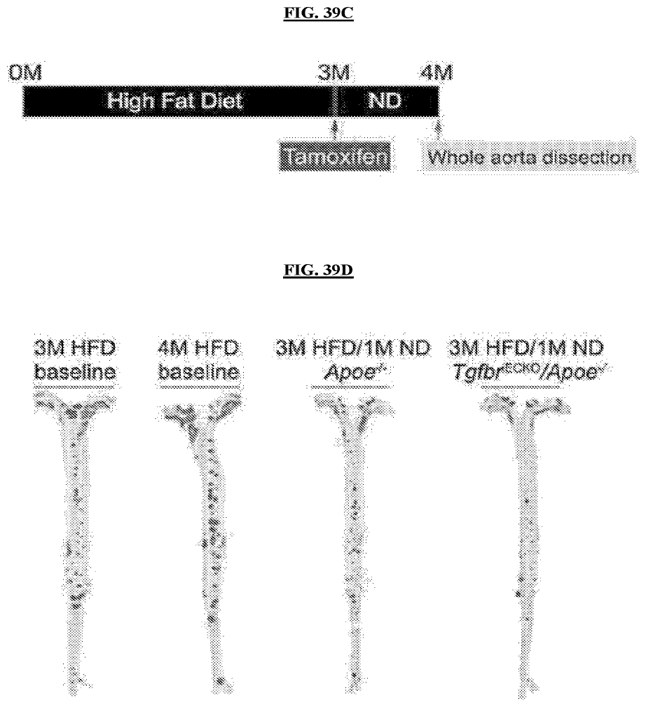

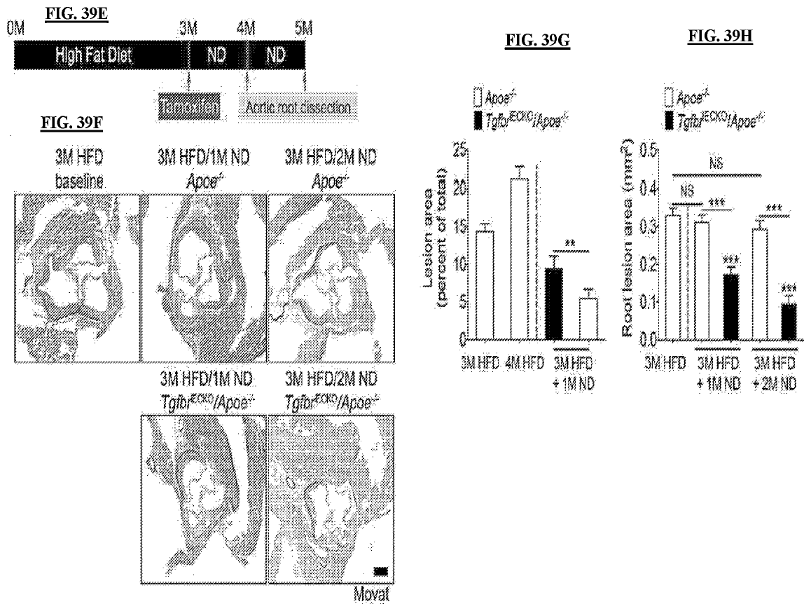

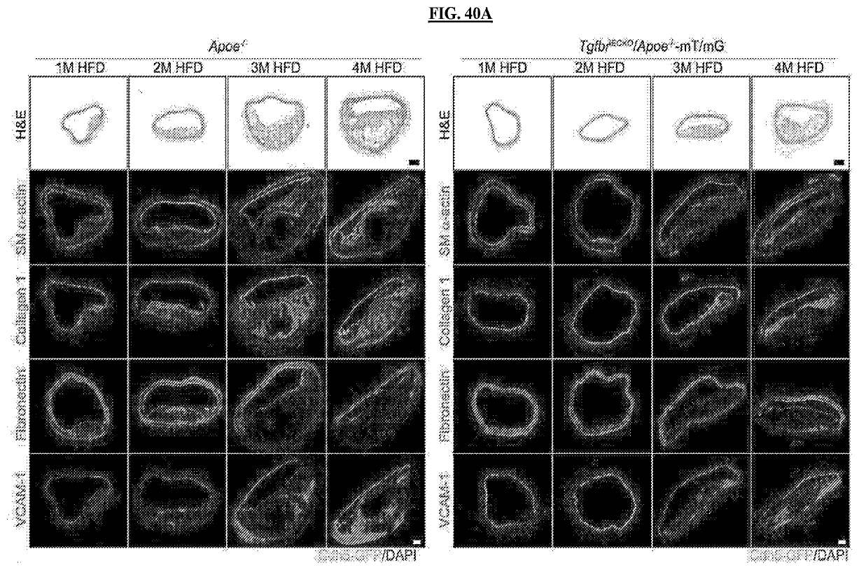

Given the profound effect of suppression of endothelial TGFβ activation on the development of atherosclerosis, a similar approach was taken to examine whether it would reduce progression and induce regression of established atherosclerotic lesions. To test this, 2 months after Cdh5CreER^{T2}; Tgfbr1^{fl/fl}; Tgfbr2^{fl/fl}; Apoe^{-/-}; mT/mG^{fl/fl} mice were placed on the HFD, the animals were randomized to tamoxifen-driven Cre activation (generating Tgfrbr^{iECKO}/Apoe^{-/-} mice) or sham treatment and continued on the HFD diet (FIG. 39A). Two months later, both groups were sacrificed and the extent of atherosclerotic burden determined using whole aorta Oil-Red-O staining. As expected, the control mice demonstrated extensive progression of disease with the total aortic lesion area increasing from 6.8% to 17%. At the same time, mice with the induced endothelial TGFβR1/R2 deletion showed no significant disease progression (6.6% to 8.5%, p=NS) (FIG. 39B). Thus, endothelial deletion of TGFβR1 and R2 arrested atherosclerosis progression in the presence of strikingly elevated cholesterol levels.

To test the effect of this intervention on lesion regression under normocholesterolemic conditions, Cdh5CreER²; Tgfbr1^{fl/fl}; Tgfbr2^{fl/fl}; Apoe^{-/-}; mT/mG^{fl/fl} mice were kept on the HFD for 3 months. At that time, they were switched to the normal chow diet and randomized to Cdh5CreER^{T2} activation, inducing endothelial-specific Tgfβr1 and Tgfβr2 deletions (FIG. 39C). While both Apoe^{-/-} and Tgfrbr^{iECKO}/Apoe^{-/-} mice showed an expected decrease in the lipid uptake, it was far more profound in the latter (FIGS. 39D&39G). Histological sections of the aortic root were used to analyze the extent of atherosclerotic plaques size and composition after one or two months of the normal chow diet (FIG. 39E). While there was no significant reduction in the aortic root plaque size in Apoe^{-/-} mice either after 1 or 2 months of the normal chow diet, Tgfrbr^{iECKO}/Apoe^{-/-} mice showed a 47% decrease in lesion size after 1 month and a 71% decrease after 2 months (FIGS. 39F&39H). Morphological analysis of atherosclerotic plaques showed expected plaque progression in Apoe^{-/-} mice. At the same time, induction of Tgfβr1/Tgfβr2 deletions resulted in significant plaque regression.

In sum, the experiments demonstrate that the luciferase control or 7C1-let-7 injection had no effect on body weight, blood cell counts, organ appearance, organ weight, serum lipid profile, and liver function. 7C1-let-7 had an effect on plaque lesion size: reduced atherosclerotic lesion in both Apoe^{-/-} and Frs2α^{iECKO}/Apoe^{-/-} mice after 4 months on a high fat diet was observed.

### Example 7: Analysis of TGFβ Signaling in FRS2α Knockdown HUVEC and Endothelial Cells from Subjects Having No or Varying Degrees of Atherosclerosis/Coronary Artery Disease

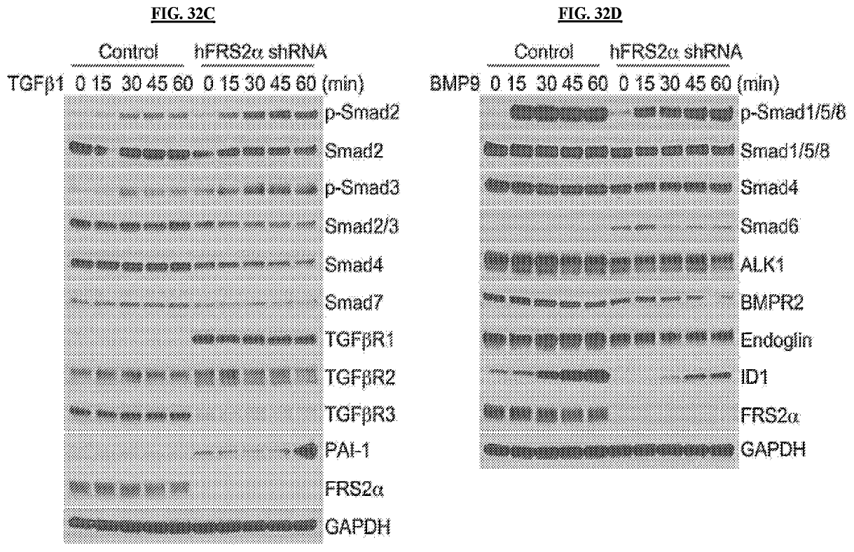

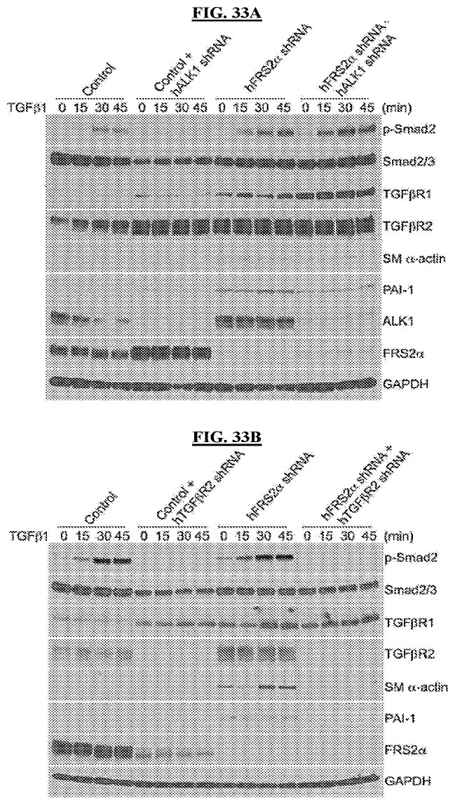

FIGS. 32A-32D are plots and blots showing TGFβ and BMP signaling in a FRS2α knockdown background. FIG. 32A shows levels of Type I, Type II, and Type III TGFβ receptors in a FRS2α knockdown background. FIG. 32B shows levels of TGFβ and BMP signaling components in a FRS2α knockdown background. FIG. 32C shows a time course of levels of TGFβ signaling components in a FRS2α knockdown background. FIG. 32D shows a time course of levels of BMP signaling components in a FRS2α knockdown background.

FIGS. 33A-33B are blots showing TGFβ and BMP signaling, respectively, in a ALK1 knockdown, TGFβR2 knockdown, FRS2α knockdown, ALK1/FRS2α knockdown, and TGFβR2/FRS2α knockdown background.

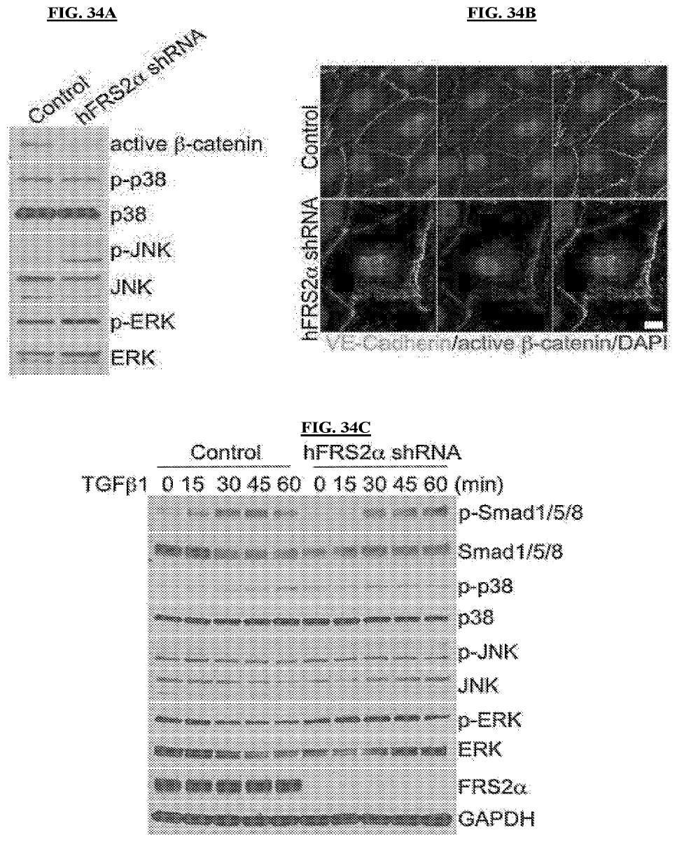

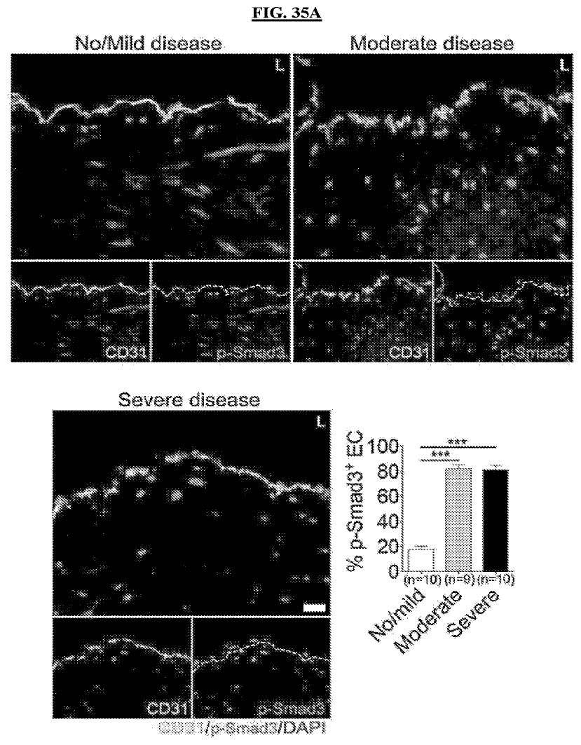

FIGS. 34A-34C are blots and an image showing MAPK signaling in a FRS2α knockdown background. FIGS. 34A and 34C show levels of MAPK signaling components in a FRS2α knockdown.

FIG. 34B shows an analysis using anti-VE cadherin and anti-active P-catenin. Nuclei were counterstained with DAPI.

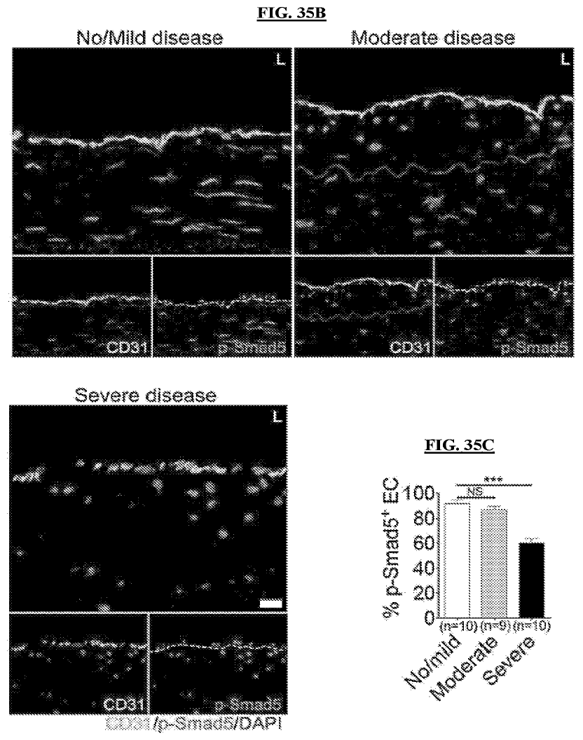

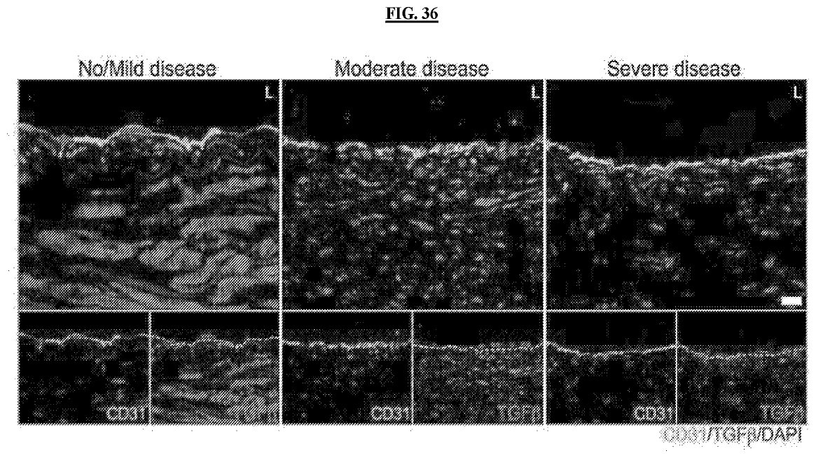

FIGS. 35A-35C are images showing TGFβ signaling activity in endothelial cells from subjects having No/mild disease, moderate disease, and severe disease, using anti-CD31, anti-p-Smad3, and anti-p-Smad5 antibodies. FIG. 35A shows immunostaining for p-Smad3. FIG. 35B shows immunostaining for p-Smad5. FIG. 35C shows quantification of immunocytochemistry data from FIG. 35B. Nuclei were counterstained with DAPI.

FIG. 36 are images showing TGFβ signaling activity in arteries from subjects having No/mild disease, moderate disease, and severe disease, using anti-CD31 and anti-TGFβ antibodies. Nuclei were counterstained with DAPI.

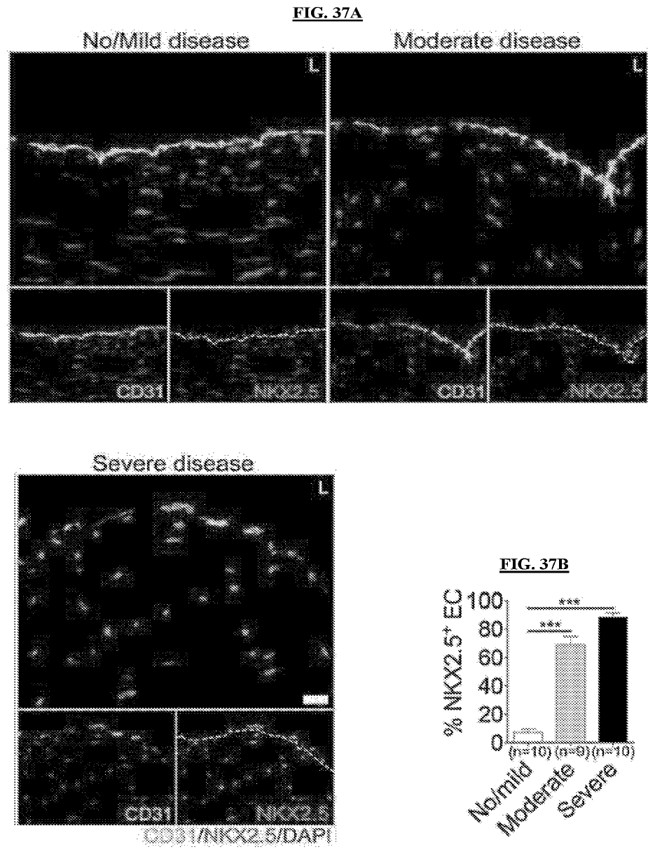

FIGS. 37A-37B are images and a plots showing NKX2.5 expression in endothelial cells from subjects having No/mild disease, moderate disease, and severe disease. Nuclei were counterstained with DAPI. FIG. 37A shows immunostaining for NKX2.5. FIG. 37B shows quantification of immunocytochemistry data from FIG. 37A.

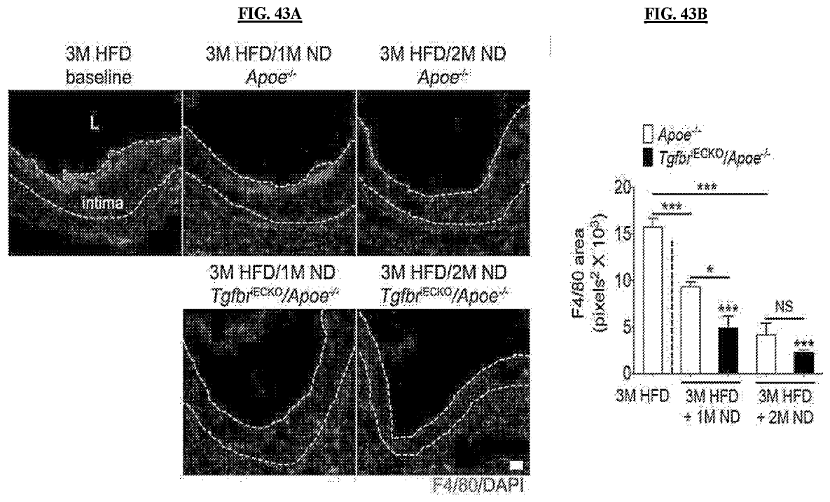

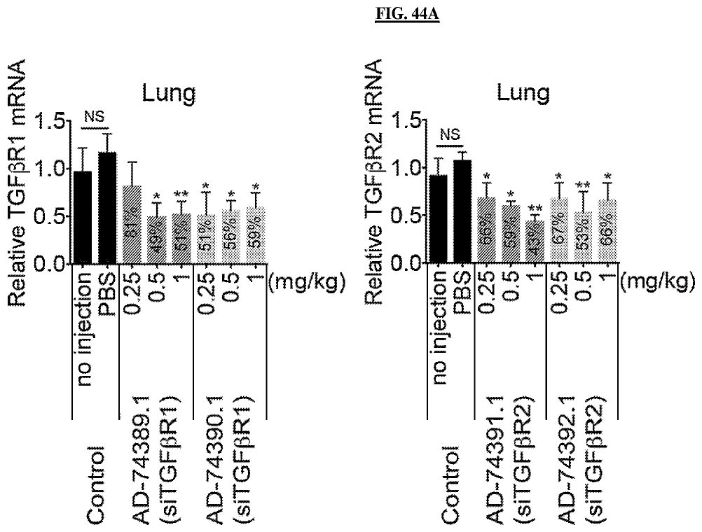

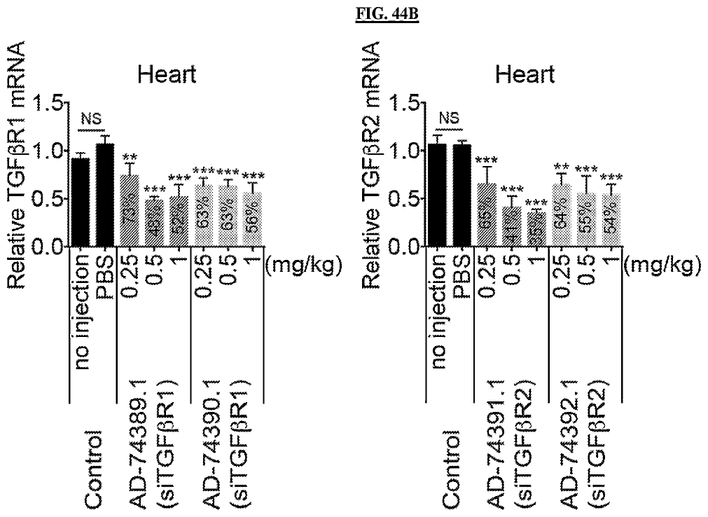

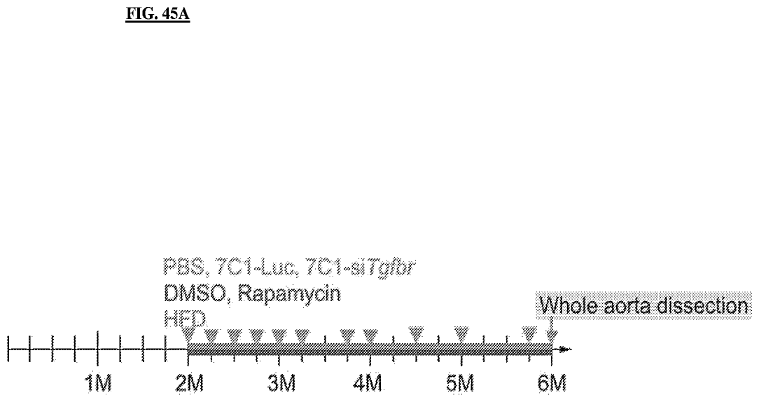

FIGS. 43A-43B are a series of images and histograms showing the effects of endothelial cell Tgfbr1/Tgfbr2 knockout on the regression of atherosclerosis macrophage content by measurement and histological analysis.

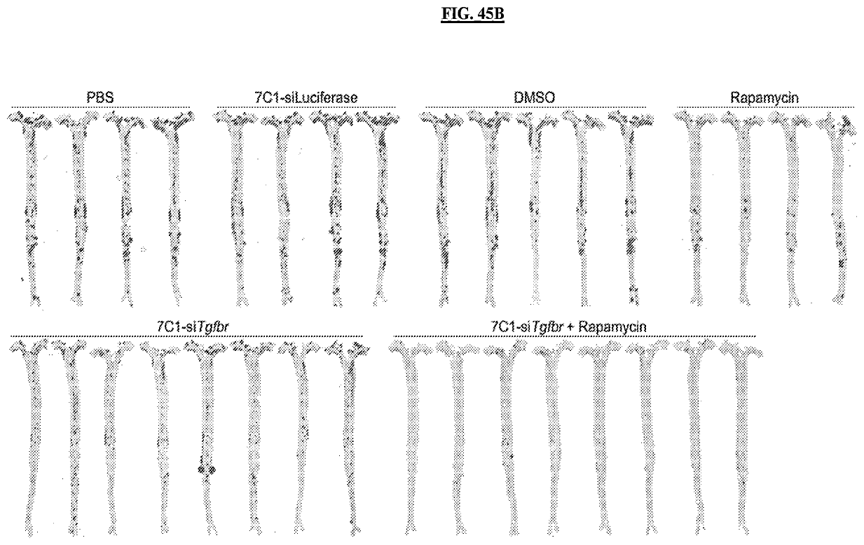

FIGS. 44A-44B are series of histograms depicting the in vivo assessment of siTgfbr1 and siTgfbr2 in heart and lung endothelial cells (EC). The Expression of Tgfbr1 and Tgfbr2 were analyzed by quantitative real-time PCR and showed that siTgfbr1(AD-74389.1) and siTgfbr2 (AD-74391.1) have great knockdown efficiency in both lung and heart.

### Example 8: The Combination of 7C1-siTgfbr and Rapamycin Provides Optimal Reduction of Atherosclerotic Lesion

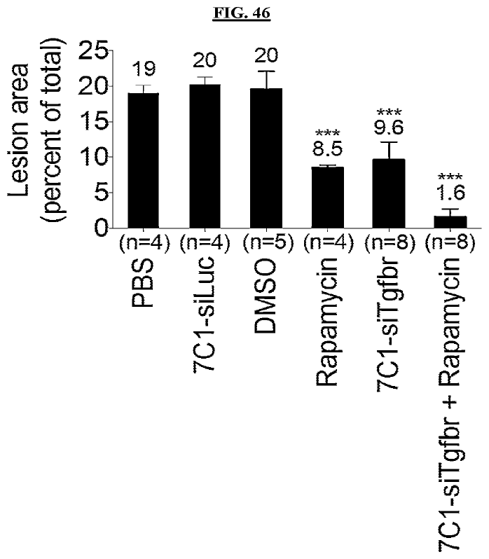

FIGS. 45A-45B are a graph and series of images showing that 7C1-siTgfbr and rapamycin suppress atherosclerosis lesion development in Apoe^{-/-} mice after 4 months of high fat diet.

FIG. 46 is a histogram illustrating the quantification of atherosclerotic lesions from FIG. 45B. The quantification of the lesion area was performed by computing the percentage of lesion area over the total area of aorta. Mice treated with 7C1-siTgfbr exhibited 52% reduction in their atherosclerotic lesion, mice treated with rapamycin exhibited 58% reduction in their atherosclerotic lesion and mice treated with a combination of both 7C1-siTgfbr and rapamycin exhibited 92% reduction in their atherosclerotic lesion. These results highlight that the combination of when 7C1-siTgfbr and rapamycin allow reaching optimal results when used for atherosclerosis treatment.

### Example 9

The results of this study show that activation of endothelial TGFβ signaling plays a key role in the development and progression of atherosclerosis. Selective inhibition of endothelial TGFβ signaling, using either Tgfβr1/Tgfβr2 deletions or nanoparticle-based let-7b miR delivery, delays the onset of the disease, reduces the rate of atherosclerosis progression in the settings of hypercholesterolemia and facilitates regression under normocholesterolemic conditions. Taken together, these data implicate endothelial TGFβ signaling as the key factor responsible for atherosclerotic plaque growth and maintenance.

TGFβ has long been recognized as an important regulator involved in a variety of biological roles including cell proliferation, differentiation, migration, adhesion, and extracellular matrix (ECM) production. Abnormal TGFβ signaling has been implicated in pathogenesis of a number diseases from systemic sclerosis and various fibrosis-associated illnesses to Marfan's syndrome, aortic aneurysms and related disorders, to inflammation-related syndromes and allergic disease among many others (Lafyatis et al, 2014 Nature reviews. Rheumatology 10, 706-719; Lan et al, 2013 Cardiovascular pathology: the official journal of the Society for Cardiovascular Pathology 22, 401-407; Pardali et al, 2012 International journal of biological sciences 8, 195-213; Frischmeyer-Guerrerio et al, 2013 Science translational medicine 5, 195ra194; Gallo et al, 2014 The Journal of clinical investigation 124, 448-460; Tedgui et al, 2006 Physiological reviews 86, 515-581).

Importantly, TGFβ signature has been detected in atherosclerosis (Schunkert et al., 2011 Nature genetics 43, 333-338) and expression of TGFβ ligands, receptors and various Smad proteins has been reported in atherosclerotic plaques (Pardali et al, 2012 International journal of biological sciences 8, 195-213; McCaffrey et al, 2009 Frontiers in bioscience 1, 236-245). However, the role of TGFβ in atherosclerosis has been controversial, with both pro- and anti-atherosclerotic effects reported (Toma et al, 2012 Cell and tissue research 347, 155-175; Tabas et al, 2015 J Cell Biol 209, 13-22). In particular, systemic inhibition of TGFβ signaling using a neutralizing anti-TGFβ1, -β2, and -β3 antibody was shown to accelerate the development of atherosclerosis in Apoe^{-/-} mice (Mallat et al, 2001 Circulation research 89, 930-934) while treatment with anti-TGFβR2 antibody decreased plaque size of advanced lesions, but increased plaque vulnerability (Lutgens et al, 2002 Arterioscler Thromb Vasc Biol 22, 975-982). Mice with TGFβR2 knockout in CD11c⁺ cells dendritic cells exhibited an increase in the plaque area (Lievens et al, 2013 European heart journal 34, 3717-3727) as did the Apoe^{-/-} mice with disrupted TGFβ signaling in T cells (Gistera et al, 2013 Science translational medicine 5, 196ra100; Robertson et al, 2003 The Journal of clinical investigation 112, 1342-1350).

Among deleterious effects of activated endothelial TGFβ signaling is the induction of endothelial to mesenchymal transition (EndMT) (van Meeteren et al, 2012 Cell and tissue research 347, 177-186). EndMT is frequently observed in human atherosclerotic lesions (Chen et al, 2015 The Journal of clinical investigation 125, 4529-4543) and its extent strongly correlates with the severity of disease. It contributes directly to atherosclerotic plaque growth due to endothelial cells acquiring smooth muscle and mesenchymal (fibroblast) fate and extensive deposition of extracellular matrix. Indeed, induction of EndMT, and hence endothelial TGFβ signaling, in mice accelerates the development of atherosclerosis and increases plaque size. EndMT is also an important driver of inflammation due to increased endothelial expression of leukocyte adhesion molecules. For these reasons, in the present invention the endothelial TGFβ signaling cascade cells were specifically targeted using genetic and molecular approaches.

Both approaches were equally effective in reducing the total lesion burden and plaque size. In addition, plaque morphology was favorably affected with a decrease in the necrotic core size implying increased plaque stability. Importantly, favorable changes were seen in multiple vascular locations including the total aortic endothelium, aortic root and brachiocephalic artery. This was driven by a reduction in the extent of EndMT, as shown by decreased number of endothelial-derived αSMA-positive cells in the plaque, and a large decrease in plaque inflammation, as documented by decreased presence of Mac3⁺ and F4/80⁺ macrophages and T and B-cells, most likely due to decreased recruitment.

In summary, this study establishes endothelial TGFβ signaling as an important driver of atherosclerotic plaque growth and demonstrates a potential utility of a therapeutic intervention aimed at suppression of this process.

TABLE 1
Human subject characteristics*

| Disease Severity by I/M Ratio | | | |
|---|---|---|---|
| No/Mild | Moderate | Severe | |
| I/M <0.2 | I/M 0.2-1.0 | I/M >1.0 | |
| 0.14 ± 0.03 | 0.4 ± 0.2 | 2.0 ± 1.4 | |
| n = 10 | n = 15 | n = 18 | P value |

Explanted Hearts

| | | | | |
|---|---|---|---|---|
| Organ donors | 6 (60.0) | 8 (53.3) 1 | 0 (55.6) | 0.9470 |
| Transplant | 4 (40.0) | 7 (46.7) | 8 (44.4) | 0.9470 |
| recipients | | | | |

Demographics

| | | | | |
|---|---|---|---|---|
| Age (yr) | 42.3 ± 13.9 | 56.7 ± 8.3 | 61.6 ± 6.5 | <0.0001 |
| Male | 4 (40.0) | 10 (66.7) | 13 (72.2) | 0.2226 |
| Caucasian | 7 (70.0) | 10 (66.7) | 15 (83.3) | 0.5149 |

Past Medical History

| | | | | |
|---|---|---|---|---|
| Coronary | 0 (0.0) | 0 (0.0) | 9 (50.0) | 0.0004 |
| artery disease | | | | |
| Cerebrovascular | 1 (10.0) | 0 (0.0) | 4 (22.2) | 0.1377 |
| disease | | | | |
| Peripheral | 1 (10.0) | 1 (6.7) | 2 (11.1) | 0.9053 |
| vascular disease | | | | |

Atherosclerosis Risk Factors

| | | | | |
|---|---|---|---|---|
| Diabetes | 2 (20.0) | 3 (20.0) | 6 (33.3) | 0.6135 |
| mellitus | | | | |
| Hypertension | 4 (40.0) | 9 (60.0) | 11 (61.1) | 0.5155 |
| Hyperlipidemia | 2 (20.0) | 3 (20.0) | 7 (46.7) | 0.3954 |
| Tobacco use | 4 (40.0) | 7 (46.7) | 10 (55.6) | 0.7168 |
| Obesity | 4 (40.0) | 2 (13.3) | 5 (27.8) | 0.3135 |

*Left main coronary arteries were procured from the explanted hearts of 43 individuals within the operating room either at organ donation or cardiac transplantation. The degree of atherosclerotic disease was quantified as intima to media (I/M) ratio and de-identified clinical data was recorded. Data represent Number (%) or Mean ± SD. Comparisons between groups of categorical variables were by Chi-square test and of continuous variables were by one-way ANOVA.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-53. (canceled)

54. A method for reducing an atherosclerotic lesion in a subject, the method comprising administering to the subject a nanoparticle formulated for selective delivery to endothelial cells, wherein the nanoparticle comprises an effective amount of siRNA, and wherein the siRNA targets mRNA encoding transforming growth factor beta (TGFβ)1, TGFβ2, TGFβ3, TGFβ receptor (TGFβR)1, or TGFβR2, and reduces the expression of endothelial TGFβ1, endothelial TGFβ2, endothelial TGFβ3, endothelial TGFβR1, or endothelial TGFβR2, respectively.

55. The method according to claim 54, wherein the siRNA targets mRNA encoding human TGFβ1, human TGFβ2, human TGFβ3, human TGFβR1, or human TGFβR2, and wherein the subject is a human subject.

56. The method according to claim 55, wherein the nanoparticle comprises siRNA that targets mRNA encoding human TGFβR1 or human TGFβR2.

57. The method according to claim 55, wherein the nanoparticle comprises siRNA that targets mRNA encoding human TGFβR1 and siRNA that targets mRNA encoding human TGFβR2.

58. The method according to claim 54, wherein the nanoparticle is intravenously administered to the subject.

59. A method for inhibiting progression of atherosclerosis or reversing atherosclerosis in a subject, the method comprising administering to the subject a nanoparticle formulated for selective delivery to endothelial cells, wherein the nanoparticle comprises an effective amount of siRNA, and wherein the siRNA targets mRNA encoding transforming growth factor beta (TGFβ)1, TGFβ2, TGFβ3, TGFβ receptor (TGFβR)1, or TGFβR2, and reduces the expression of endothelial TGFβ1, endothelial TGFβ2, endothelial TGFβ3, endothelial TGFβR1, or endothelial TGFβR2, respectively.

60. The method according to claim 59, wherein the siRNA targets mRNA encoding human TGFβ1, human TGFβ2, human TGFβ3, human TGFβR1, or human TGFβR2, and wherein the subject is a human subject.

61. The method according to claim 60, wherein the nanoparticle comprises siRNA that targets mRNA encoding human TGFβR1 or human TGFβR2.

62. The method according to claim 60, wherein the nanoparticle comprises siRNA that targets mRNA encoding human TGFβR1 and siRNA that targets mRNA encoding human TGFβR2.

63. The method according to claim 59, wherein the nanoparticle is intravenously administered to the subject.

64. A method for treating atherosclerosis in a subject, the method comprising administering to the subject a nanoparticle formulated for selective delivery to endothelial cells, wherein the nanoparticle comprises an effective amount of siRNA, and wherein the siRNA targets mRNA encoding transforming growth factor beta (TGFβ)1, TGFβ2, TGFβ3, TGFβ receptor (TGFβR)1, or TGFβR2, and reduces the expression of endothelial TGFβ1, endothelial TGFβ2, endothelial TGFβ3, endothelial TGFβR1, or endothelial TGFβR2, respectively.

65. The method according to claim 64, wherein the siRNA targets mRNA encoding human TGFβ1, human TGFβ2, human TGFβ3, human TGFβR1, or human TGFβR2, and wherein the subject is a human subject.

66. The method according to claim 65, wherein the nanoparticle comprises siRNA that targets mRNA encoding human TGFβR1 or human TGFβR2.

67. The method according to claim 65, wherein the nanoparticle comprises siRNA that targets mRNA encoding human TGFβR1 and siRNA that targets mRNA encoding human TGFβR2.

68. The method according to claim 64, wherein the nanoparticle is intravenously administered to the subject.


SEQUENCE LISTING (38 sequences)

SEQ ID NO: 1 - DNA, 24 bp, synthetic construct
  misc_feature 1..24: note = Primer
  source 1..24: mol_type = other DNA
gagtgtgctgtgattggaaggcag

SEQ ID NO: 2 - DNA, 24 bp, synthetic construct
  misc_feature 1..24: note = Primer
  source 1..24: mol_type = other DNA
ggcacgagtgtctgcagacacatg

SEQ ID NO: 3 - DNA, 20 bp, synthetic construct
  misc_feature 1..20: note = Primer
  source 1..20: mol_type = other DNA
gtctggcagtaaaaactatc

SEQ ID NO: 4 - DNA, 23 bp, synthetic construct
  misc_feature 1..23: note = Primer
  source 1..23: mol_type = other DNA
gtgaaacagcattgctgtcactt

SEQ ID NO: 5 - DNA, 24 bp, synthetic construct
  misc_feature 1..24: note = Primer
  source 1..24: mol_type = other DNA
ctaggccacagaattgaaagatct

SEQ ID NO: 6 - DNA, 25 bp, synthetic construct
  misc_feature 1..25: note = Primer
  source 1..25: mol_type = other DNA
gtaggtggaaattctagcatcatcc

SEQ ID NO: 7 - DNA, 20 bp, synthetic construct
  misc_feature 1..20: note = Primer
  source 1..20: mol_type = other DNA
gcctagccgagggagagccg

SEQ ID NO: 8 - DNA, 21 bp, synthetic construct
  misc_feature 1..21: note = Primer
  source 1..21: mol_type = other DNA
gtgacttgggagctctgcagc

SEQ ID NO: 9 - DNA, 18 bp, synthetic construct
  misc_feature 1..18: note = Primer
  source 1..18: mol_type = other DNA
gccgccccgactgcatct

SEQ ID NO: 10 - DNA, 26 bp, synthetic construct
  misc_feature 1..26: note = Primer
  source 1..26: mol_type = other DNA
gcctgcattaccggtcgatgcaacga

SEQ ID NO: 11 - DNA, 26 bp, synthetic construct
  misc_feature 1..26: note = Primer
  source 1..26: mol_type = other DNA
gtggcagatggcgcggcaacaccatt

SEQ ID NO: 12 - DNA, 24 bp, synthetic construct
  misc_feature 1..24: note = Primer
  source 1..24: mol_type = other DNA
actcacatgttggctctcactgtc

SEQ ID NO: 13 - DNA, 24 bp, synthetic construct
  misc_feature 1..24: note = Primer
  source 1..24: mol_type = other DNA
agtcatagagcatgtgttagagtc

SEQ ID NO: 14 - DNA, 20 bp, synthetic construct
  misc_feature 1..20: note = Primer
  source 1..20: mol_type = other DNA
taaacaaggtccggagccca

SEQ ID NO: 15 - DNA, 20 bp, synthetic construct
  misc_feature 1..20: note = Primer
  source 1..20: mol_type = other DNA
acttctgcaagaggtcccct

SEQ ID NO: 16 - DNA, 21 bp, synthetic construct
  misc_feature 1..21: note = Primer
  source 1..21: mol_type = other DNA
ctctgctgcctcctggcttct

SEQ ID NO: 17 - DNA, 21 bp, synthetic construct
  misc_feature 1..21: note = Primer
  source 1..21: mol_type = other DNA
cgaggcggatcacaagcaata

SEQ ID NO: 18 - DNA, 19 bp, synthetic construct
  misc_feature 1..19: note = Primer
  source 1..19: mol_type = other DNA
tcaatgggcgggggtcgtt

SEQ ID NO: 19 - RNA, 22 nt, synthetic construct
  misc_feature 1..22: note = Primer
  source 1..22: mol_type = other RNA
tgaggtagtaggttgtgtggtt

SEQ ID NO: 20 - RNA, 22 nt, synthetic construct
  misc_feature 1..22: note = Primer
  source 1..22: mol_type = other RNA
ctatacaacctactgccttccc

SEQ ID NO: 21 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 22 - protein, 3913 aa, Homo sapiens
AGCGCTCTTGCGGCCACAGGCGCGGCGTCCTCGGCGGCGGGCGGCAGCTAGCGGGAGCCGGGACGCCGGTGCAGCCGCAGCGCGCGGAGGAACCCGGGTGTGCCGGGAGCTGGGCGGCCACGTCCGGACGGGACCGAGACCCCTCGTAGCGCATTGCGGCGACCTCGCCTTCCCCGGCCGCGAGCGCGCCGCTGCTTGAAAAGCCGCGGAACCCAAGGACTTTTCTCCGGTCCGAGCTCGGGGCGCCCCGCAGGGCGCACGGTACCCGTGCTGCAGTCGGGCACGCCGCGGCGCCGGGGCCTCCGCAGGGCGATGGAGCCCGGTCTGCAAGGAAAGTGAGGCGCCGCCGCTGCGTTCTGGAGGAGGGGGGCACCAGCTCCGGCTCCATTGTTCCCGCCCGGGCTGGAGGCGCCGAGCACCGAGCGCCGCCGGGAGTCGAGCGCCGGCCGCGGAGCTCTTGCGACCCCGCCAGGACCCGAACAGAGCCCGGGGGCGGCGGGCCGGAGCCGGGGACGCGGGCACACGCCCGCTCGCACAAGCCACGGCGGACTCTCCCGAGGCGGAACCTCCACGCCGAGCGAGGGTCAGTTTGAAAAGGAGGATCGAGCTCACTGTGGAGTATCCATGGAGATGTGGAGCCTTGTCACCAACCTCTAACTGCAGAACTGGGATGTGGAGCTGGAAGTGCCTCCTCTTCTGGGCTGTGCTGGTCACAGCCACACTCTGCACCGCTAGGCCGTCCCCGACCTTGCCTGAACAAGCCCAGCCCTGGGGAGCCCCTGTGGAAGTGGAGTCCTTCCTGGTCCACCCCGGTGACCTGCTGCAGCTTCGCTGTCGGCTGCGGGACGATGTGCAGAGCATCAACTGGCTGCGGGACGGGGTGCAGCTGGCGGAAAGCAACCGCACCCGCATCACAGGGGAGGAGGTGGAGGTGCAGGACTCCGTGCCCGCAGACTCCGGCCTCTATGCTTGCGTAACCAGCAGCCCCTCGGGCAGTGACACCACCTACTTCTCCGTCAATGTTTCAGATGCTCTCCCCTCCTCGGAGGATGATGATGATGATGATGACTCCTCTTCAGAGGAGAAAGAAACAGATAACACCAAACCAAACCGTATGCCCGTAGCTCCATATTGGACATCCCCAGAAAAGATGGAAAAGAAATTGCATGCAGTGCCGGCTGCCAAGACAGTGAAGTTCAAATGCCCTTCCAGTGGGACCCCAAACCCCACACTGCGCTGGTTGAAAAATGGCAAAGAATTCAAACCTGACCACAGAATTGGAGGCTACAAGGTCCGTTATGCCACCTGGAGCATCATAATGGACTCTGTGGTGCCCTCTGACAAGGGCAACTACACCTGCATTGTGGAGAATGAGTACGGCAGCATCAACCACACATACCAGCTGGATGTCGTGGAGCGGTCCCCTCACCGGCCCATCCTGCAAGCAGGGTTGCCCGCCAACAAAACAGTGGCCCTGGGTAGCAACGTGGAGTTCATGTGTAAGGTGTACAGTGACCCGCAGCCGCACATCCAGTGGCTAAAGCACATCGAGGTGAATGGGAGCAAGATTGGCCCAGACAACCTGCCTTATGTCCAGATCTTGAAGACTGCTGGAGTTAATACCACCGACAAAGAGATGGAGGTGCTTCACTTAAGAAATGTCTCCTTTGAGGACGCAGGGGAGTATACGTGCTTGGCGGGTAACTCTATCGGACTCTCCCATCACTCTGCATGGTTGACCGTTCTGGAAGCCCTGGAAGAGAGGCCGGCAGTGATGACCTCGCCCCTGTACCTGGAGATCATCATCTATTGCACAGGGGCCTTCCTCATCTCCTGCATGGTGGGGTCGGTCATCGTCTACAAGATGAAGAGTGGTACCAAGAAGAGTGACTTCCACAGCCAGATGGCTGTGCACAAGCTGGCCAAGAGCATCCCTCTGCGCAGACAGGTGTCTGCTGACTCCAGTGCATCCATGAACTCTGGGGTTCTTCTGGTTCGGCCATCACGGCTCTCCTCCAGTGGGACTCCCATGCTAGCAGGGGTCTCTGAGTATGAGCTTCCCGAAGACCCTCGCTGGGAGCTGCCTCGGGACAGACTGGTCTTAGGCAAACCCCTGGGAGAGGGCTGCTTTGGGCAGGTGGTGTTGGCAGAGGCTATCGGGCTGGACAAGGACAAACCCAACCGTGTGACCAAAGTGGCTGTGAAGATGTTGAAGTCGGACGCAACAGAGAAAGACTTGTCAGACCTGATCTCAGAAATGGAGATGATGAAGATGATCGGGAAGCATAAGAATATCATCAACCTGCTGGGGGCCTGCACGCAGGATGGTCCCTTGTATGTCATCGTGGAGTATGCCTCCAAGGGCAACCTGCGGGAGTACCTGCAGGCCCGGAGGCCCCCAGGGCTGGAATACTGCTACAACCCCAGCCACAACCCAGAGGAGCAGCTCTCCTCCAAGGACCTGGTGTCCTGCGCCTACCAGGTGGCCCGAGGCATGGAGTATCTGGCCTCCAAGAAGTGCATACACCGAGACCTGGCAGCCAGGAATGTCCTGGTGACAGAGGACAATGTGATGAAGATAGCAGACTTTGGCCTCGCACGGGACATTCACCACATCGACTACTATAAAAAGACAACCAACGGCCGACTGCCTGTGAAGTGGATGGCACCCGAGGCATTATTTGACCGGATCTACACCCACCAGAGTGATGTGTGGTCTTTCGGGGTGCTCCTGTGGGAGATCTTCACTCTGGGCGGCTCCCCATACCCCGGTGTGCCTGTGGAGGAACTTTTCAAGCTGCTGAAGGAGGGTCACCGCATGGACAAGCCCAGTAACTGCACCAACGAGCTGTACATGATGATGCGGGACTGCTGGCATGCAGTGCCCTCACAGAGACCCACCTTCAAGCAGCTGGTGGAAGACCTGGACCGCATCGTGGCCTTGACCTCCAACCAGGAGTACCTGGACCTGTCCATGCCCCTGGACCAGTACTCCCCCAGCTTTCCCGACACCCGGAGCTCTACGTGCTCCTCAGGGGAGGATTCCGTCTTCTCTCATGAGCCGCTGCCCGAGGAGCCCTGCCTGCCCCGACACCCAGCCCAGCTTGCCAATGGCGGACTCAAACGCCGCTGACTGCCACCCACACGCCCTCCCCAGACTCCACCGTCAGCTGTAACCCTCACCCACAGCCCCTGCTGGGCCCACCACCTGTCCGTCCCTGTCCCCTTTCCTGCTGGCAGGAGCCGGCTGCCTACCAGGGGCCTTCCTGTGTGGCCTGCCTTCACCCCACTCAGCTCACCTCTCCCTCCACCTCCTCTCCACCTGCTGGTGAGAGGTGCAAAGAGGCAGATCTTTGCTGCCAGCCACTTCATCCCCTCCCAGATGTTGGACCAACACCCCTCCCTGCCACCAGGCACTGCCTGGAGGGCAGGGAGTGGGAGCCAATGAACAGGCATGCAAGTGAGAGCTTCCTGAGCTTTCTCCTGTCGGTTTGGTCTGTTTTGCCTTCACCCATAAGCCCCTCGCACTCTGGTGGCAGGTGCCTTGTCCTCAGGGCTACAGCAGTAGGGAGGTCAGTGCTTCGTGCCTCGATTGAAGGTGACCTCTGCCCCAGATAGGTGGTGCCAGTGGCTTATTAATTCCGATACTAGTTTGCTTTGCTGACCAAATGCCTGGTACCAGAGGATGGTGAGGCGAAGGCCAGGTTGGGGGCAGTGTTGTGGCCCTGGGGCCCAGCCCCAAACTGGGGGCTCTGTATATAGCTATGAAGAAAACACAAAGTGTATAAATCTGAGTATATATTTACATGTCTTTTTAAAAGGGTCGTTACCAGAGATTTACCCATCGGGTAAGATGCTCCTGGTGGCTGGGAGGCATCAGTTGCTATATATTAAAAACAAAAAAAAAAAAAAA

SEQ ID NO: 23 - protein, 508 aa, Homo sapiens
MGSCCSCPDKDTVPDNHRNKFKVINVDDDGNELGSGIMELTDTELILYTRKRDSVKWHYLCLRRYGYDSNLFSFESGRRCQTGQGIFAFKCARAEELFNMLQEIMQNNSINVVEEPVVERNNHQTELEVPRTPRTPTTPGFAAQNLPNGYPRYPSFGDASSHPSSRHPSVGSARLPSVGEESTHPLLVAEEQVHTYVNTTGVQEERKNRTSVHVPLEARVSNAESSTPKEEPSSIEDRDPQILLEPEGVKFVLGPTPVQKQLMEKEKLEQLGRDQVSGSGANNTEWDTGYDSDERRDAPSVNKLVYENINGLSIPSASGVRRGRLTSTSTSDTQNINNSAQRRTALLNYENLPSLPPVWEARKLSRDEDDNLGPKTPSLNGYHNNLDPMHNYVNTENVTVPASAHKIEYSRRRDCTPTVFNFDIRRPSLEHRQLNYIQVDLEGGSDSDNPQTPKTPTTPLPQTPTRRTELYAVIDIERTAAMSNLQKALPRDDGTSRKTRHNSTDLPM

SEQ ID NO: 24 - DNA, 6676 bp, Homo sapiens
  source 1..6676: mol_type = other DNA
aaaacccttccctcccccgctcccccggaagtgcttttccaagattcgggccggagagaggccttgtaggcacagcggctgagactcgatctgctccaagtaggggctccagcgcgggtcggagtctgggggttcgcgcccgccgacccgcgccctgctccctctcagcacctgggcggacggttaaatcagcaaacaaagaaaacatggtattttgaaatatgattaaactcctgatgctgcagcagaggctaagaatattaatggccagatctagtgcacacatggtcttctgaagaagccatgggtagctgttgtagctgtccagataaagacactgtcccagataaccatcggaacaagtttaaggtcattaatgtggatgatgatgggaatgagttaggttctggcataatggaacttacagacacagaactgattttatacacccgcaaacgtgactcagtaaaatggcactacctctgcctgcgacgctatggctatgactcgaatctcttttcttttgaaagtggtcgaaggtgtcaaactggacaaggaatctttgcctttaagtgtgcccgtgcagaagaattatttaacatgttgcaagagattatgcaaaataatagtataaatgtggtggaagagccagttgtagaaagaaataatcatcagacagaattggaagtccctagaacacctcgaacacctacaactccaggatttgctgctcagaacttacctaatggatatccccgatatccctcatttggagatgcttcatcccatccgtcaagcagacatccttctgtgggaagtgctcgcctgccttcagtaggggaagaatctacacatcctttgcttgtggctgaggaacaagtacatacctatgtcaacactacaggtgtgcaagaagagcggaaaaaccgcacaagtgtgcatgttccattggaggcgagggtttctaacgctgaaagcagcacaccaaaagaagaaccaagtagtattgaggacagggatcctcagattcttcttgaacctgaaggagtcaaatttgttttagggccaacccctgttcaaaagcagttaatggaaaaagagaaactggagcaacttggaagagatcaagttagtggaagtggagcaaataacacagaatgggacactggctatgacagtgatgaacgaagagatgcaccctctgttaacaaactggtgtatgaaaatataaatgggctatctatccctagtgcctcaggggtcaggagaggtcgtctgacatccaccagtacctcagatacccagaatatcaacaactcagctcagagaagaactgcattattaaactatgaaaatctaccatctttgcctcctgtttgggaagcccgcaagctaagtagggatgaagatgacaatttaggaccaaagaccccatctctaaatggctaccataataatctagatccaatgcataactatgtaaatacagagaatgtaacagtgccagcaagtgctcacaaaatagaatattcaaggcgtcgggactgtacaccaacagtctttaactttgatatcagacgcccaagtttagaacacaggcagcttaattacatacaggttgacttggaaggtggcagtgactctgacaaccctcagactccaaaaacgcctacaactccccttccacaaacccctaccaggcgcacagagctgtatgccgtgatagacatcgagagaactgctgctatgtcaaatttgcagaaagcactgccacgagatgatggtacatctaggaaaactagacacaatagtactgatctgcccatgtgagcctggaaagcattgtgttgtttgcacctttgtgaagtttttaaaaatgaagatgcaagtgcttcattttcatttctaaacactaactccttttatagactgataaaatttttttctgaatatttcatgtgcatctttaactaaagggaattaatgtagagcaggtactccttaaagaacactaatttcattatatactactcgttgtacagcagcattcccgttttcacagtgcctatttaaaatgagagttgaagtaaatgacatgctggttgatttttatcaatattctggacttaacgcatacctttcatgtctaagtcatggttggcttttaaaactttttataaagcctcttgacaatgtacattgctaacaggtaactataggctttgaaagtaatgctcgtagattcagtgttcacagtatgtggcctccagcatgtaacatgaggaatcctttatttcattaattaatggctttttgacttgagccaaaacatatgtaaaggaaacagaagtaccgcacctcctcttacaccagtcagctcctttgccttcagtgttactagaaagcggcctgtgtccatgagtgtgctttgctgttggtgcactgaaaggcaggaaggagacaagattttctatttactcatctcatgatgtcatttgaagggcatgtccagatatcttaaaattataataggctcaagaatcagtctcaggtcactttacccaaaaacatttgaaaatctgaaccacaatctcctgaaagtttttctcctatagattgttgacaacacattgttttctggaggcatttgtgccattaggtttccatttatcttcagtttttttctttggtgtttgggatgtcttattttgttgccttatgtccttttcaatttaaaatgtttgagtttgtatatagttttgaaattggattatgtgttcattgttgtttagtttgcatttttgtcaaattatggttttgaaggttcatttggaacttactgttagtctgtaacagggttgcccttgtccagtatttatttataagctgtttacttttcaagttgataaaaacattctccaattctaaatttgcttgtgtccataggtgatctctttagcaaactgagaaaaaaaggaagctacttttaacatgcaaagttccctcaaggtgtaccgtgttgtctctgtgggcactcttccccagcactttagcagtaattcccccagctacacgctgcagttgtactctgcccactctagtgttcctcagctctgctgtccttttacttgtagctggatctttgattatccttcgatttccatgaaatattaatattgttgccagcatagcaggtacagtggaagtcttgtagcagtgagattgtatcataatttaggatttaaaatgaattaaagtttatataaactgaagagtctccatatgtcaaactcttggaaaatcaaagatgttccaatttcctaaacactagagaatacgagagaaggtagagtggaaaaggttaggtaaccttgcaaaatattttactattttctctaaatatgaggaagtttgagattatgatctggatctaccagatataactaaggttaatttagcatgaaaaagttttagtcatattggcatccaacctattcagtaaccgaatcataggacaatgatggattaggagaacaatagagtgggatcattataaagaaaataaattattaaaggtgtctttatcgttttagtgccatttttagtgtctttactataaatcaatatcagtgtattttatcattctatgtgcatagcagaattttcttttctcccttttgttcccctgtgaacttggtgcttattaaagtgctcactgttctcttaaaagagagcagtggtataggtgtgcagtttccatgatgcaggttccatttttaatatattgttccacttatcctttcttctgagtaaattgctaattgtgccaaatttatgtaatagtttttgtaatgtggaataagaattatgatggaaccattgcacatttttttctgaaacagccagtcaaggcagaacattaatctccaaatgcaagggctgatctatttattcattttggaggttgggtactttattctttctttccgtcatccttttcattgtttcccccggattctaattagtttttattttttttagataactccaatataatcattacagtttatgctttaaatactatgtgctttaaaaaggaaaatgggaccaatttgtctgctaagaatttgattttaggtactataagagtattaggaaaatatatacaactggtgttaatttctagatattttctagaaatcacttgtgttcctatttaataaaaggtaatttagaatactacttgtcctttgcagtagtttagtaatgggcattaagctgtgtcctcgaaggatgtacctattactaggtgcattttagaatgaaatattgatattttattagcatataattgtggccatatatctcagattttctgaggcagatctaattttagataattctgttggtagaccatgtgatccttctttttggttttggaaatataatcattgttaatgttttccctccaaatagaatactgttttatccatacaaatcataacagcatctatcccatgctagggttggaaactgatattggtattacttgtgttttttcttagtgtgttttatttcccagtttcatcttcttctaaaaatgaaaatatggtgccttccctccctccaggaagactggcaaatatttccttttatttactgctgctgtggagtgatgagatatgcactttactctttaagattcagcaaaaagcttttcacttctcagtatatccagaatacatcatatctgggacttaggaaaatttgccaagcaatctttgtttttatagatactaatgttgaccctctccagcgttcaatgttataaatagaacaagtcaagctagtgtttatctcctccccctccccaaaactgtggcacagcatataaaaatgtacctcaataatgttctattaaaaatgggacaggggccttatgttttcataatttcccaacaatgtgccgccatatttttgcctcaaggtaaaggttttaacagatgaaaaagtacttcccaattcccccgtgctattcctaacctataatgcccaaatgttttgtgcaatgtgtagtgtgtgtgtataaatacatatattcttgaaatagacataccatcagagacatcattcacaagtaactgatgtattggcatctcattcatatttctgatgtgtgaggtatatggtactaattaccttttccttgatgtttgccaaatttgaataaaggcattggtacgaaattacagaatgtaaagaaaatgtttttggcttgaaaaattaacatattttatgacgtaccacagtatactctgcccaaaccagcaccctatctatctttcctgttctttacatccctgttccccatccctacttcctcatttttggtataacacagttcttttgtagcatcattataattgcagttctatggcaattggacagttatagcatggaaacagactggtataagtagtacagtagtcaccagtgtgccacatttgcattagtaatgcaaaatatacattttataaaggacaaactttgtgttatgttttattttcattacattgtataatattgtaagactattgtatgtcctaatttgcattataaatgtttttttcctacgtaaaggcataaatatagcaactttgtataaaggtagcttattagatttttaattttttcttttataaaaaattgtccaacagtgggactaccattgccaaattgtatatgaaatatgaattttacccccatggttaatttcttttataaacattccatatttctctaataaaaagacataagtgatactgtactatgcatacattgtatcttaatgctgtttcagatcagcattttaaattttggtttgcatttttaatattggcaaaacgtaaccactgttaattaaaataaaaccttgttgtatatgtaacaacataattttccctctatcccttcccaccctttgttctctatttctccctatcagtgccaacttcatacattttgtagcatggcaataaaatataacttttacactgaggccgagtgtggctttttggaggaagtggggatgggacgattgccctctagttgtcctttgcatatgactgttttttgccatataagccatgtcatcaggcatgaaaagttttctcatatatgatgtaaacttgcttttaaggacaagtgtgaatgtgctttttaagcttaatttttgtcatgacaactaattttttttatctttggagaagtcagagttctttacaatcaaacgtttattaactggagtacttagaataagctagtaattgaatttagttcaagggctaagcaacacatttttaaatccttatttattgtagagtattagtatactgtcctacaaattatgtaaaatatggtttaatattagatgactttggattttgcaatgccttactgttgtcattctagcataaatatccataatgaggtactcaagttgatactggaagctgagctgatcatacactgacctgaagcattcatgaaaagctgctttattgaataaagtctgattggagttcttttcatgctcactttccccttattgctgaaagtagattgcaataaaaccccaataaaacgtttggtcggatatctacttaaaaaaaaaaaa

SEQ ID NO: 25 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 26 - DNA, 1746 bp, Homo sapiens
  source 1..1746: mol_type = other DNA
cccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggaaaaaaaaaaaaaaaaaaaaaaaaaaaaaaaaaaaaaaaaaaaaaaaaaaaaaaaaaaaaaaaaaaa

SEQ ID NO: 27 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 28 - DNA, 2570 bp, Homo sapiens
  source 1..2570: mol_type = other DNA
gcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcag

SEQ ID NO: 29 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 30 - DNA, 930 bp, Homo sapiens
  source 1..930: mol_type = other DNA
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtag

SEQ ID NO: 31 - protein, 426 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 32 - DNA, 5766 bp, Homo sapiens
  source 1..5766: mol_type = other DNA
gcggcggctagggaggtggggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaattctacagctttgcctgaactctccttttttcttcagatctgctcctgggttttaatttgggaggtcaattgttctacctcactgagagggaacagaaggatattgcttccttttgcagcagtgtaataaagtcaattaaaaacttcccaggatttctttggacccaggaaacagccatgtgggtcctttctgtgcactatgaacgcttctttcccaggacagaaaatgtgtagtctacctttattttttattaacaaaacttgttttttaaaaagatgattgctggtcttaactttaggtaactctgctgtgctggagatcatctttaagggcaaaggagttggattgctgaattacaatgaaacatgtcttattactaaagaaagtgatttactcctggttagtacattctcagaggattctgaaccactagagtttccttgattcagactttgaatgtactgttctatagtttttcaggatcttaaaactaacacttataaaactcttatcttgagtctaaaaatgacctcatatagtagtgaggaacataattcatgcaattgtattttgtatactattattgttctttcacttattcagaacattacatgccttcaaaatgggattgtactataccagtaagtgccacttctgtgtctttctaatggaaatgagtagaattgctgaaagtctctatgttaaaacctatagtgtttgaattcaaaaagcttatttatctgggtaacccaaactttttctgttttgtttttggaagggtttttgtggtatgtcatttggtattctattctgaaaatgcctttctcctaccaaaatgtgcttaagccactaaagaaatgaagtggcattaattagtaaattattagcatggtcatgtttgaatattctcacatcaagcttttgcattttaattgtgttgtctaagtatacttttaaaaaatcaagtggcactctagatgcttatagtactttaatatttgtagcatacagactaatttttctaaaagggaaagtctgtctagctgcttgtgaaaagttatgtggtattctgtaagccatttttttctttatctgttcaaagacttattttttaagacatgaattacatttaaaattagaatatggttaatattaaataataggcctttttctaggaaggcgaaggtagttaataatttgaatagataacagatgtgcaagaaagtcacatttgttatgtatgtaggagtaaacgttcggtggatcctctgtctttgtaactgaggttagagctagtgtggttttgaggtctcactacactttgaggaaggcagcttttaattcagtgtttccttatgtgtgcgtacattgcaactgcttacatgtaatttatgtaatgcattcagtgcacccttgttacttgggagaggtggtagctaaagaacattctgagtataggtttttctccatttacagatgtctttggtcaaatattgaaagcaaacttgtcatggtcttcttacattaagttgaaactagcttataataactggtttttacttccaatgctatgaagtctctgcagggcttttacagttttcgaagtccttttatcactgtgatcttattctgaggggagaaaaaactatcatagctctgaggcaagacttcgactttatagtgctatcagttccccgatacagggtcagagtaacccatacagtattttggtcaggaagagaaagtggccatttacactgaatgagttgcattctgataatgtcttatctcttatacgtagaataaatttgaaagactatttgatcttaaaaccaaagtaattttagaatgagtgacatattacataggaatttagtgtcaatttcatgtgtttaaaaacatcatgggaaaaatgcttagaggttactattttgactacaaagttgagtttttttctgtagttaccataatttcattgaagcaaatgaatgagtttgagaggtttgtttttatagttgtgttgtattacttgtttaataataatctctaattctgtgatcaggtactttttttgtgggggttttttttttgtttttttttttttttgttgttgtttttgggccatttctaagcctaccagatctgctttatgaaatccaggggaccaatgcattttatcactaaaactatttttatataattttaagaatataccaaaagttgtctgatttaaagttgtaatacatgatttctcactttcatgtaaggttatccacttttgctgaagatattttttattgaatcaaagattgagttacaattatacttttcttacctaagtggataaaatgtacttttgatgaatcagggaatttttttaaagttggagtttagttctaaattgactttacgtattactgcagttaattccttttttggctagggatggtttgataaaccacaattggctgatattgaaaatgaaagaaacttaaaaggtgggatggatcatgattactgtcgataactgcagataaatttgattagagtaataattttgtcatttaaaaacacagttgtttatactgcccatcctaggatgctcaccttccaagattcaacgtggctaaaacatcttctggtaaattgtgcgtccatattcattttgtcagtagccaggagaaatggggatgggggaaatacgacttagtgaggcatagacatccctggtccatcctttctgtctccagctgtttcttggaacctgctctcctgcttgctggtccctgacgcagagaccgttgcctcccccacagccgtttgactgaaggctgctctggagacctagagtaaaacggctgatggaagttgtgggacccacttccatttccttcagtcattagaggtggaagggaggggtctccaagtttggagattgagcagatgaggcttgggatgcccctgctttgacttcagccatggatgaggagtgggatggcagcaaggtggctcctgtggcagtggagttgtgccagaaacagtggccagttgtatcgcctataagacagggtaaggtctgaagagctgagcctgtaattctgctgtaataatgatagtgctcaagaagtgccttgagttggtgtacagtgccatggccatcaagaatcccagatttcaggttttattacaaaatgtaagtggtcacttggcgattttgtagtacatgcatgagttaccttttttctctatgtctgagaactgtcagattaaaacaagatggcaaagagatcgttagagtgcacaacaaaatcactatcccattagacacatcatcaaaagcttatttttattcttgcactggaagaatcgtaagtcaactgtttcttgaccatggcagtgttctggctccaaatggtagtgattccaaataatggttctgttaacactttggcagaaaatgccagctcagatattttgagatactaaggattatctttggacatgtactgcagcttcttgtctctgttttggattactggaatacccatgggccctctcaagagtgctggacttctaggacattaagatgattgtcagtacattaaacttttcaatcccattatgcaatcttgtttgtaaatgtaaacttctaaaaatatggttaataacattcaacctgtttattacaacttaaaaggaacttcagtgaatttgtttttattttttaacaagatttgtgaactgaatatcatgaaccatgttttgatacccctttttcacgttgtgccaacggaatagggtgtttgatatttcttcatatgttaaggagatgcttcaaaatgtcaattgctttaaacttaaattacctctcaagagaccaaggtacatttacctcattgtgtatataatgtttaatatttgtcagagcattctccaggtttgcagttttatttctataaagtatgggtattatgttgctcagttactcaaatggtactgtattgtttatatttgtaccccaaataacatcgtctgtactttctgttttctgtattgtatttgtgcaggattctttaggctttatcagtgtaatttctgccttttaagatatgtacagaaaatgtccatataaatttccattgaagtcgaatgatactgagaagcctgtaaagaggagaaaaaacataagctgtgtttccccataagtttttttaaattgtatattgtatttgtagtaatattccaaaagaatgtaaataggaaatagaagagtgatgcttatgttaagtcctaacactacagtagaagaatggaagcagtgcaaataaattacatttttcccaaaaaaaaaaaaaaaaaaaaaaaaaaaaaaagaaaaaaaaaaaaaaa

SEQ ID NO: 33 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 34 - DNA, 487 bp, Homo sapiens
  source 1..487: mol_type = other DNA
cctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcgggtgagtggtccccagcccgggctcggcggggcgccgggggtcttcctggggtccccgcctctccgctgcgcttgacagtcgggcccggcaacccggcccccgggcggaaacgaggaaagtttcccccgcgacactcacgcagcccgactcccgtagctgcagggattgtgagtttttcttgaaaaagagaaggaaagttcagttgcaaggggcgcggggcacgtttggtcc

SEQ ID NO: 35 - RNA, 23 nt, synthetic construct
  source 1..23: mol_type = other RNA
tgtcaaggagatgcttcaatatt

SEQ ID NO: 36 - RNA, 25 nt, synthetic construct
  source 1..25: mol_type = other RNA
tattgaagcatctccttgacatatt

SEQ ID NO: 37 - RNA, 23 nt, synthetic construct
  source 1..23: mol_type = other RNA
ggctcgctgaacactaccaaatt

SEQ ID NO: 38 - RNA, 25 nt, synthetic construct
  source 1..25: mol_type = other RNA
tttggtagtgttcagcgagccattt